SEQUENCE LISTING (46 sequences)

SEQ ID NO: 1 - protein, 464 aa, Oryza brachyantha
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 2 - protein, 464 aa, synthetic construct
MASGRSSASAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 3 - protein, 464 aa, synthetic construct
MASGRSSARAAGMMHVVICPWLAFGHLLPCLDLAQRLASRGHRVSFVSTPRNLSRLPPVRPALASVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 4 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWNSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 5 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDNPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 6 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGSSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 7 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNTGDGSFDREGVAAVVRAVAVAEESRRVFRANAKKLQEIVADMACHDGYIDGFIQQLKSYKD

SEQ ID NO: 8 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQIPNARLIQAKKAGLQVPRNDGDGSFDREGVAAVVRAVAVAEESRRVFRANAKKLQEIVADMACHDGYIDGFIQQLKSYKD

SEQ ID NO: 9 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNAFLIQAKKAGLQVPRNDGDGSFDREGVAAVVRAVAVAEESRRVFRANAKKLQEIVADMACHDGYIDGFIQQLKSYKD

SEQ ID NO: 10 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKASSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 11 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNARLIQAKKAGLQVPRNDGDGSFDREGVAAVVRAVAVAEESRRVFRANAKKLQEIVADMACHDGYIDGFIQQLKSYKD

SEQ ID NO: 12 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAAPRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 13 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNSGDGSFDREGVAAVVRAVAVAEESRRVFRANAKKLQEIVADMACHDGYIDGFIQQLKSYKD

SEQ ID NO: 14 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNANLIQAKKAGLQVPRNDGDGSFDREGVAAVVRAVAVAEESRRVFRANAKKLQEIVADMACHDGYIDGFIQQLKSYKD

SEQ ID NO: 15 - protein, 464 aa, synthetic construct
MASGRSSARAAGMMHVVICPWLAFGHLLPCLDLAQRLASRGHRVSFVSTPRNLSRLPPVRPALAPRVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 16 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNAHLIQAKKAGLQVPRNDGDGSFDREGVAAVVRAVAVAEESRRVFRANAKKLQEIVADMACHDGYIDGFIQQLKSYKD

SEQ ID NO: 17 - protein, 464 aa, synthetic construct
MASGRSSARAAGMMHVVICPWLAFGHLLPCLDLAQRLASRGHRVSFVSTPRNLSRLPPVRPALAPFVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 18 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNDGDGSFDREGVAAVVRAVAVAEESRRVFRANAKKLQEIVADMACHDGYIDGFIQQLKSYKD

SEQ ID NO: 19 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 20 - protein, 464 aa, synthetic construct
MASNRSSARAAGMMHVVICPWLAFGHLLPCLDLAQRLASRGHRVSFVSTPRNLSRLPPVRPALAPVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKDSSGMSLAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNARLIQAKKAGLQVPRNDGDGSFDREGVAAVVRAVAVAEESRRVFRANAKKLQEIVADMACHDGYIDGFIQQLKSYKD

SEQ ID NO: 21 - protein, 464 aa, synthetic construct
MASGRSSARAAGMMHVVICPWLAFGHLLPCLDLAQRLASRGHRVSFVSTPRNLSRLPPVRPALASFVSFVALPLPRVEGLPDGAESTNDVPQDRPDMVELHRIAFDGLGSSFSEFLRTASADWVIVDVFHHWGSAAAVEHKVPCAMLLLSSAHMISSISERRPESAESPAAAGEGRPAAAPTFEAAPRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNADLIQAKKAGLQVPRNTGDGSFDREGVAAVVRAVAVAEESRRVFRANAKKLQEIVADMACHDGYIDGFIQQLKSYKD

SEQ ID NO: 22 - protein, 464 aa, synthetic construct
MASGRSSASAAGMMHVVICPWLAFGHLLPCLDLAQRLASRGHRVSFVSTPRNLSRLPPVRPALASVVSFVALPLPRVEGLPDGAESTNDVPQDRPDMVELHRIAFDGLGSSFSEFLRTASADWVIVDVFHHWGSAAAVEHKVPCAMLLLSSAHMISSISERRPESAESPAAAGEGRPAAAPTFEAAPRKLIRKKDSSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNDGDGSFDREGVAAVVRAVAVAEESRRVFRANAKKLQEIVADMACHDGYIDGFIQQLKSYKD

SEQ ID NO: 23 - protein, 464 aa, synthetic construct
MASGRSSARAAGMMHVVICPWLAFGHLLPCLDLAQRLASRGHRVSFVSTPRNLSRLPPVRPALASVVSFVALPLPRVEGLPDGAESTNDVPQDRPDMVELHRIAFDGLGSSFSEFLRTASADWVIVDVFHHWGSAAAVEHKVPCAMLLLSSAHMISSISERRPESAESPAAAGEGRPAAAPTFEAAPRKLIRKKDSSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNADLIQAKKAGLQVPRNTGDGSFDREGVAAVVRAVAVAEESRRVFRANAKKLQEIVADMACHDGYIDGFIQQLKSYKD

SEQ ID NO: 24 - protein, 464 aa, synthetic construct
MASNRSSARAAGMMHVVICPWLAFGHLLPCLDLAQRLASRGHRVSFVSTPRNLSRLPPVRPALAPVVSFVALPLPRVEGLPDGAESTNDVPQDNPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNADLIQAKKAGLQVPRNDGDGSFDREGVAAVVRAVAVAEESRRVFRANAKKLQEIVADMACHDGYIDGFIQQLKSYKD

SEQ ID NO: 25 - protein, 464 aa, synthetic construct
MASNRSSARAAGMMHVVICPWLAFGHLLPCLDLAQRLASRGHRVSFVSTPRNLSRLPPVRPALAPVVSFVALPLPRVEGLPDGAESTNDVPQDNPDMVELHRIAFDGLGVSFSEFLRTASADWVIVDVFHHWGSAAAVEHKVPCAMLLLSSAHMISSISERRPESAESPAAAGEGRPAAAPTFEAAPRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNTGDGSFDREGVAAVVRAVAVAEESRRVFRANAKKLQEIVADMACHDGYIDGFIQQLKSYKD

SEQ ID NO: 26 - protein, 464 aa, synthetic construct
MASGRSSARAAGMMHVVICPWLAFGHLLPCLDLAQRLASRGHRVSFVSTPRNLSRLPPVRPALAPVVSFVALPLPRVEGLPDGAESTNDVPQDNPDMVELHRIAFDGLGVSFSEFLRTASADWVIVDVFHHWGSAAAVEHKVPCAMLLLSSAHMISSISERRPESAESPAAAGEGRPAAAPTFEAAPRKLIRKKDSSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNTGDGSFDREGVAAVVRAVAVAEESRRVFRANAKKLQEIVADMACHDGYIDGFIQQLKSYKD

SEQ ID NO: 27 - protein, 464 aa, synthetic construct
MASNRSSARAAGMMHFVICPWLAFGHLLPCLDLAQRLASRGHRVSFVSTPRNLSRLPPVRPALAPVVSFVALPLPRVEGLPDGAESTNDVPQDNPDMVELHRIAFDGLGSSFSEFLRTASADWVIVDVFHHWGSAAAVEHKVPCAMLLLSSAHMISSISERRPESAESPAAAGEGRPAAAPTFEAARRKLIRKKDSSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNDGDGSFDREGVAAVVRAVAVAEESRRVFRANAKKLQEIVADMACHDGYIDGFIQQLKSYKD

SEQ ID NO: 28 - protein, 464 aa, synthetic construct
MASNRSSASAAGMMHVVICPWLAFGHLLPCLDLAQRLASRGHRVSFVSTPRNLSRLPPVRPALASVVSFVALPLPRVEGLPDGAESTNDVPQDRPDMVELHRIAFDGLGVSFSEFLRTASADWVIVDVFHHWGSAAAVEHKVPCAMLLLSSAHMISSISERRPESAESPAAAGEGRPAAAPTFEAAPRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQGPNADLIQAKKAGLQVPRNTGDGSFDREGVAAVVRAVAVAEESRRVFRANAKKLQEIVADMACHDGYIDGFIQQLKSYKD

SEQ ID NO: 29 - protein, 464 aa, synthetic construct
MASGRSSASAAGMMHVVICPWLAFGHLLPCLDLAQRLASRGHRVSFVSTPRNLSRLPPVRPALAPVVSFVALPLPRVEGLPDGAESTNDVPQDNPDMVELHRIAFDGLGVSFSEFLRTASADWVIVDVFHHWGSAAAVEHKVPCAMLLLSSAHMISSISERRPESAESPAAAGEGRPAAAPTFEAARRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNADLIQAKKAGLQVPRNTGDGSFDREGVAAVVRAVAVAEESRRVFRANAKKLQEIVADMACHDGYIDGFIQQLKSYKD

SEQ ID NO: 30 - protein, 464 aa, synthetic construct
MASGRSSARAAGMMHVVICPWLAFGHLLPCLDLAQRLASRGHRVSFVSTPRNLSRLPPVRPALASVVSFVALPLPRVEGLPDGAESTNDVPQDNPDMVELHRIAFDGLGSSFSEFLRTASADWVIVDVFHHWGSAAAVEHKVPCAMLLLSSAHMISSISERRPESAESPAAAGEGRPAAAPTFEAARRKLIRKKASSGMSNAERFFLTLSRSNLVVVRSCAELEPETVPLLSTVRGKPVAFLGLMPPSPDGRRGGVSHEGGEDDPVRWLDAQPAESVVYVALGSEAPLLVEKVHELALGLELAGTRFLWALRKPAGVSDADLLPAGFRERTGGRGLVATRWVPQLSILAHAAVGAFLTHCGWSSTIEGLMFGRPLIMLPISGDQHPNADLIQAKKAGLQVPRNDGDGSFDREGVAAVVRAVAVAEESRRVFRANAKKLQEIVADMACHDGYIDGFIQQLKSYKD

SEQ ID NO: 31 - protein, 464 aa, Oryza brachyantha
MENGSSPLHVVIFPWLAFGHLLPFLDLAERLAARGHRVSFVSTPRNLARLRPVRPALRGLVDLVALPLPRVHGLPDGAEATSDVPFEKFELHRKAFDGLAAPFSAFLDAACAGDKRPDWVIPDFMHYWVAAAAQKRGVPCAVLIPCSADVMALYGQPTETSTEQPEAIARSMAAEAPSFEAERNTEEYGTAGASGVSIMTRFSLTLKWSKLVALRSCPELEPGVFTTLTRVYSKPVVPFGLLPPRRDGAHGVRKNGEDDGAIIRWLDEQPAKSVVYVALGSEAPVSADLLRELAHGLELAGTRFLWALRRPAGVNDGDSILPNGFLERTGERGLVTTGWVPQVSILAHAAVCAFLTHCGWGSVVEGLQFGHPLIMLPIIGDQGPNARFLEGRKVGVAVPRNHADGSFDRSGVAGAVRAVAVEEEGKAFAANARKLQEIVADRERDERCTDGFIHHLTSWNELEA

SEQ ID NO: 32 - protein, 463 aa, Hordeum vulgare
MDGDGNSSSSSSPLHVVICPWLALGHLLPCLDIAERLASRGHRVSFVSTPRNIARLPPLRPAVAPLVEFVALPLPHVDGLPEGAESTNDVPYDKFELHRKAFDGLAAPFSEFLRAACAEGAGSRPDWLIVDTFHHWAAAAAVENKVPCVMLLLGAATVIAGFARGVSEHAAAAVGKERPAAEAPSFETERRKLMTTQNASGMTVAERYFLTLMRSDLVAIRSCAEWEPESVAALTTLAGKPVVPLGLLPPSPEGGRGVSKEDAAVRWLDAQPAKSVVYVALGSEVPLRAEQVHELALGLELSGARFLWALRKPTDAPDAAVLPPGFEERTRGRGLVVTGWVPQIGVLAHGAVAAFLTHCGWNSTIEGLLFGHPLIMLPISSDQGPNARLMEGRKVGMQVPRDESDGSFRREDVAATVRAVAVEEDGRRVFTANAKKMQEIVADGACHERCIDGFIQQLRSYKA

SEQ ID NO: 33 - protein, 462 aa, Oryza sativa
MDSGYSSSYAAAAGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMMLLGSAHMIASIADRRLERAETESPAAAGQGRPAAAPTFEVARMKLIRTKGSSGMSLAERFSLTLSRSSLVVGRSCVEFEPETVPLLSTLRGKPITFLGLMPPLHEGRREDGEDATVRWLDAQPAKSVVYVALGSEVPLGVEKVHELALGLELAGTRFLWALRKPTGVSDADLLPAGFEERTRGRGVVATRWVPQMSILAHAAVGAFLTHCGWNSTIEGLMFGHPLIMLPIFGDQGPNARLIEAKNAGLQVARNDGDGSFDREGVAAAIRAVAVEEESSKVFQAKAKKLQEIVADMACHERYIDGFIQQLRSYKD

SEQ ID NO: 34 - protein, 460 aa, synthetic construct
MDSGYSSSAAGGMHVVICPWLAFGHLLPCLDLAQRLASRGHRVSFVSTPRNISRLPPVRPALAPLVAFVALPLPRVEGLPDGAESTNDVPHDRPDMVELHRRAFDGLAAPFSEFLGTACADWVIVDVFHHWAAAAALEHKVPCAMILLGSAHMVASLADRRLERAETESPAVAGQGRPAAAPTFEVARMKLIRTKGSSGMSLAERFSLTLSRSSLVVVRSCAEFEPETVPLLSTLRGKPLAFLGLMPPSHEGRREDGEDDTVRWLDAQPAKSVVYVALGSEVPLRVEKVHELALGLELAGTRFLWALRKPSGVSDADLLPAGFEERTRGRGVVATRWVPQMSILAHAAVGAFLTHCGWNSTIEGLMFGHPLIMLPIFGDQGPNARLMEAKNAGVQVPRNDGDGSFDREGVTAAIRAVAVEKESSRVFQANAKKLQVIVADMACHEGYIDGFIQQLRSYKD

SEQ ID NO: 35 - protein, 473 aa, Stevia rebaudiana
MATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEVPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNTRAVAIDHES

SEQ ID NO: 36 - protein, 481 aa, Stevia rebaudiana
MDAMATTEKKPHVIFIPFPAQSHIKAMLKLAQLLHHKGLQITFVNTDFIHNQFLESSGPHCLDGAPGFRFETIPDGVSHSPEASIPIRESLLRSIETNFLDRFIDLVTKLPDPPTCIISDGFLSVFTIDAAKKLGIPVMMYWTLAACGFMGFYHIHSLIEKGFAPLKDASYLTNGYLDTVIDWVPGMEGIRLKDFPLDWSTDLNDKVLMFTTEAPQRSHKVSHHIFHTFDELEPSIIKTLSLRYNHIYTIGPLQLLLDQIPEEKKQTGITSLHGYSLVKEEPECFQWLQSKEPNSVVYVNFGSTTVMSLEDMTEFGWGLANSNHYFLWIIRSNLVIGENAVLPPELEEHIKKRGFIASWCSQEKVLKHPSVGGFLTHCGWGSTIESLSAGVPMICWPYSWDQLTNCRYICKEWEVGLEMGTKVKRDEVKRLVQELMGEGGHKMRNKAKDWKEKARIAIAPNGSSSLNIDKMVKEITVLARN

SEQ ID NO: 37 - protein, 460 aa, Stevia rebaudiana
MAEQQKIKKSPHVLLIPFPLQGHINPFIQFGKRLISKGVKTTLVTTIHTLNSTLNHSNTTTTSIEIQAISDGCDEGGFMSAGESYLETFKQVGSKSLADLIKKLQSEGTTIDAIIYDSMTEWVLDVAIEFGIDGGSFFTQACVVNSLYYHVHKGLISLPLGETVSVPGFPVLQRWETPLILQNHEQIQSPWSQMLFGQFANIDQARWVFTNSFYKLEEEVIEWTRKIWNLKVIGPTLPSMYLDKRLDDDKDNGFNLYKANHHECMNWLDDKPKESVVYVAFGSLVKHGPEQVEEITRALIDSDVNFLWVIKHKEEGKLPENLSEVIKTGKGLIVAWCKQLDVLAHESVGCFVTHCGFNSTLEAISLGVPVVAMPQFSDQTTNAKLLDEILGVGVRVKADENGIVRRGNLASCIKMIMEEERGVIIRKNAVKWKDLAKVAVHEGGSSDNDIVEFVSELIKA

SEQ ID NO: 38 - protein, 485 aa, synthetic construct
MYNVTYHQNSKAMATSDSIVDDRKQLHVATFPWLAFGHILPYLQLSKLIAEKGHKVSFLSTTRNIQRLSSHISPLINVVQLTLPRVQELPEDAEATTDVHPEDIPYLKKASDGLQPEVTRFLEQHSPDWIIYDYTHYWLPSIAASLGISRAHFSVTTPWAIAYMGPSADAMINGSDGRTTVEDLTTPPKWFPFPTKVCWRKHDLARLVPYKAPGISDGYRMGLVLKGSDCLLSKCYHEFGTQWLPLLETLHQVPVVPVGLLPPEIPGDEKDETWVSIKKWLDGKQKGSVVYVALGSEVLVSQTEVVELALGLELSGLPFVWAYRKPKGPAKSDSVELPDGFVERTRDRGLVWTSWAPQLRILSHESVCGFLTHCGSGSIVEGLMFGHPLIMLPIFGDQPLNARLLEDKQVGIEIPRNEEDGCLTKESVARSLRSVVVEKEGEIYKANARELSKIYNDTKVEKEYVSQFVDYLEKNARAVAIDHES

SEQ ID NO: 39 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTKASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 40 - protein, 435 aa, synthetic construct
MDASSSPLHIVIFPWLAFGHMLASLELAERLAARGHRVSFVSTPRNISRLRPVPPALAPLIDFVALPLPRVDGLPDGAEATSDIPPGKTELHLKALDGLAAPFAAFLDAACADGSTNKVDWLFLDNFQYWAAAAAADHKIPCALNLTFAASTSAEYGVPRVEPPVDGSTASILQRFVLTLEKCQFVIQRACFELEPEPLPLLSDIFGKPVIPYGLVPPCPPAEGHKREHGNAALSWLDKQQPESVLFIALGSEPPVTVEQLHEIALGLELAGTTFLWALKKPNGLLLEADGDILPPGFEERTRDRGLVAMGWVPQPIILAHSSVGAFLTHGGWASTIEGVMSGHPMLFLTFLDEQRINAQLIERKKAGLRVPRREKDGSYDRQGIAGAIRAVMCEEESKSVFAANAKKMQEIVSDRNCQEKYIDELIQRLGSFEK

SEQ ID NO: 41 - protein, 320 aa, Blakeslea trispora
MLTSSKSIESFPKNVQPYGKHYQNGLEPVGKSQEDILLEPFHYLCSNPGKDVRTKMIEAFNAWLKVPKDDLIVITRVIEMLHSASLLIDDVEDDSVLRRGVPAAHHIYGTPQTINCANYVYFLALKEIAKLNKPNMITIYTDELINLHRGQGMELFWRDTLTCPTEKEFLDMVNDKTGGLLRLAVKLMQEASQSGTDYTGLVSKIGIHFQVRDDYMNLQSKNYADNKGFCEDLTEGKFSFPIIHSIRSDPSNRQLLNILKQRSSSIELKQFALQLLENTNTFQYCRDFLRVLEKEAREEIKLLGGNIMLEKIMDVLSVNE

SEQ ID NO: 42 - protein, 736 aa, Oryza sativa
MEHARPPQGGDDDVAASTSELPYMIESIKSKLRAARNSLGETTVSAYDTAWIALVNRLDGGGERSPQFPEAIDWIARNQLPDGSWGDAGMFIVQDRLINTLGCVVALATWGVHEEQRARGLAYIQDNLWRLGEDDEEWMMVGFEITFPVLLEKAKNLGLDINYDDPALQDIYAKRQLKLAKIPREALHARPTTLLHSLEGMENLDWERLLQFKCPAGSLHSSPAASAYALSETGDKELLEYLETAINNFDGGAPCTYPVDNFDRLWSVDRLRRLGISRYFTSEIEEYLEYAYRHLSPDGMSYGGLCPVKDIDDTAMAFRLLRLHGYNVSSSVFNHFEKDGEYFCFAGQSSQSLTAMYNSYRASQIVFPGDDDGLEQLRAYCRAFLEERRATGNLRDKWVIANGLPSEVEYALDFPWKASLPRVETRVYLEQYGASEDAWIGKGLYRMTLVNNDLYLEAAKADFTNFQRLSRLEWLSLKRWYIRNNLQAHGVTEQSVLRAYFLAAANIFEPNRAAERLGWARTAILAEAIASHLRQYSANGAADGMTERLISGLASHDWDWRESNDSAARSLLYALDELIDLHAFGNASDSLREAWKQWLMSWTNESQGSTGGDTALLLVRTIEICSGRHGSAEQSLKNSEDYARLEQIASSMCSKLATKILAQNGGSMDNVEGIDQEVDVEMKELIQRVYGSSSNDVSSVTRQTFLDVVKSFCYVAHCSPETIDGHISKVLFEDVN

SEQ ID NO: 43 - protein, 757 aa, Picea glauca
MKREQYTILNEKESMAEELILRIKRMFSEIENTQTSASAYDTAWVAMVPSLDSSQQPQFPQCLSWIIDNQLLDGSWGIPYLIIKDRLCHTLACVIALRKWNAGNQNVETGLRFLRENIEGIVHEDEYTPIGFQIIFPAMLEEARGLGLELPYDLTPIKLMLTHREKIMKGKAIDHMHEYDSSLIYTVEGIHKIVDWNKVLKHQNKDGSLFNSPSATACALMHTRKSNCLEYLSSMLQKLGNGVPSVYPINLYARISMIDRLQRLGLARHFRNEIIHALDDIYRYWMQRETSREGKSLTPDIVSTSIAFMLLRLHGYDVPADVFCCYDLHSIEQSGEAVTAMLSLYRASQIMFPGETILEEIKTVSRKYLDKRKENGGIYDHNIVMKDLRGEVEYALSVPWYASLERIENRRYIDQYGVNDTWIAKTSYKIPCISNDLFLALAKQDYNICQAIQQKELRELERWFADNKFSHLNFARQKLIYCYFSAAATLFSPELSAARVVWAKNGVITTVVDDFFDVGGSSEEIHSFVEAVRVWDEAATDGLSENVQILFSALYNTVDEIVQQAFVFQGRDISIHLREIWYRLVNSMMTEAQWARTHCLPSMHEYMENAEPSIALEPIVLSSLYFVGPKLSEEIICHPEYYNLMHLLNICGRLLNDIQGCKREAHQGKLNSVTLYMEENSGTTMEDAIVYLRKTIDESRQLLLKEVLRPSIVPRECKQLHWNMMRILQLFYLKNDGFTSPTEMLGYVNAVIVDPIL

SEQ ID NO: 44 - protein, 499 aa, Pisum sativum
MDTLTLSLGFLSLFLFLFLLKRSTHKHSKLSHVPVVPGLPVIGNLLQLKEKKPHKTFTKMAQKYGPIFSIKAGSSKIIVLNTAHLAKEAMVTRYSSISKRKLSTALTILTSDKCMVAMSDYNDFHKMVKKHILASVLGANAQKRLRFHREVMMENMSSKFNEHVKTLSDSAVDFRKIFVSELFGLALKQALGSDIESIYVEGLTATLSREDLYNTLVVDFMEGAIEVDWRDFFPYLKWIPNKSFEKKIRRVDRQRKIIMKALINEQKKRLTSGKELDCYYDYLVSEAKEVTEEQMIMLLWEPIIETSDTTLVTTEWAMYELAKDKNRQDRLYEELLNVCGHEKVTDEELSKLPYLGAVFHETLRKHSPVPIVPLRYVDEDTELGGYHIPAGSEIAINIYGCNMDSNLWENPDQWIPERFLDEKYAQADLYKTMAFGGGKRVCAGSLQAMLIACTAIGRLVQEFEWELGHGEEENVDTMGLTTHRLHPLQVKLKPRNRIY

SEQ ID NO: 45 - protein, 711 aa, Arabis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 46 - protein, 500 aa, synthetic construct
MEASYLYISILLLLASYLFTTQLRRKSANLPPTVFPSIPIIGHLYLLKKPLYRTLAKIAAKYGPILQLQLGYRRVLVISSPSAAEECFTNNDVIFANRPKTLFGKIVGGTSLGSLSYGDQWRNLRRVASIEILSVHRLNEFHDIRVDENRLLLRKLRDSSSPVTLRTVFYALTLNVIMRMISGKRYFDSGDRELEEEGKRFREILDETLLLAGASNVGDYLPILNWLGVKSDEKKLIALQKKRDDFFQGLIEQVRKSRGAKVGKGRKTMIELLLSLQESEPEYYTDAMIRSFVLGLLAAGSDTSAGTMEWAMSLLVNHPHVLKKAQAEIDRVVGNNRLIDESDIGNIPYLGCIINETLRLYPAGPLLFPHESSADCVISGYNIPRGTMLIVNQWAIHHDPKVWDDPETFKPERFQGLEGTRDGFKLMPFGSGRRGCPGEGLAIRLLGMTLGSVIQCFDWERVGDEMVDMTEGLGVTLPKAVPLVAKCKPRSEMTNLLSEL

(19) United States
(12) Patent Application Publication
(10) Pub. No.: US 2025/0320538 A1
(43) Pub. Date: Oct. 16, 2025

(54) COMPOSITIONS AND METHODS FOR IMPROVED PRODUCTION OF STEVIOL GLYCOSIDES

(71) Applicant: Amyris, Inc., Emeryville, CA (US)

(72) Inventors: Svetlana BORISOVA, Emeryville, CA (US); Kyle HOGAN, Emeryville, CA (US); Alexander KILBO, Emeryville, CA (US); Gale WICHMANN, Emeryville, CA (US); Yi XIONG, Emeryville, CA (US)

(21) Appl. No.: 18/866,760

(22) PCT Filed: May 18, 2023
(86) PCT No.: PCT/US2023/067184

§ 371 Date: Nov. 18, 2024

Related U.S. Application Data
(60) Provisional application No. 63/343,883, filed on May 19, 2022.

(51) Int. Cl. C12P 19/56; C07H 15/24; C12N 9/02; C12N 9/10; C12N 9/88; C12N 9/90; C12N 15/52

(52) U.S. Cl. CPC C12P 19/56; C07H 15/24; C12N 9/0042; C12N 9/0073; C12N 9/1051; C12N 9/1085; C12N 9/88; C12N 9/90; C12N 15/52; C12Y 106/02004; C12Y 114/13088; C12Y 205/01029; C12Y 402/03019; C12Y 505/01013

(57) ABSTRACT

Provided herein are variant uridine-5′-diphosphate glycosyltransferase polypeptides capable of producing steviol glycosides, yeast cells capable of producing steviol glycosides, and methods of making such cells. Also provided are fermentation compositions including the disclosed host cells, and related methods of producing and recovering steviol glycosides generated by the yeast cells.

### SEQUENCE LISTING

The instant application contains a Sequence Listing which has been submitted electronically in XML format and is hereby incorporated by reference in its entirety. Said XML copy, created on Apr. 12, 2023, is named 51494-023WO2_Sequence_Listing_4_12_23 and is 62,407 bytes in size.

### BACKGROUND OF THE INVENTION

Reduced-calorie sweeteners derived from natural sources are desired to limit the health effects of high-sugar consumption. The stevia plant (Stevia rebaudiana Bertoni) produces a variety of sweet-tasting glycosylated diterpenes termed steviol glycosides. Of all the known steviol glycosides, RebM has the highest potency (˜300 times sweeter than sucrose) and tends to have the most appealing flavor profile. However, RebM is only produced in minute quantities by the stevia plant and is a small fraction of the total steviol glycoside content (<1.0%), making the isolation of RebM from stevia leaves impractical. Alternative methods of obtaining RebM are needed. One such approach is the application of synthetic biology to design microorganisms (e.g., yeast) that produce large quantities of RebM, and other steviol glycosides, from sustainable feedstock sources.

However, producing steviol glycosides using synthetic biology remains challenging, as increased bioconversion from the feedstock to the steviol glycoside product is required. As a result, there remains a need for improved compositions and methods for making these products in host cell.

### SUMMARY OF THE INVENTION

The present disclosure provides variant uridine-5′-diphosphate (UDP) glycosyltransferase polypeptides, nucleic acids encoding the same, host cells expressing such polypeptides, and methods for production of steviol glycosides in a host cell, such as a yeast cell. The variant UDP glycosyltransferase polypeptides described herein exhibit advantageous enzymatic properties, as these polypeptides contain modifications, such as amino acid substitutions relative to a wild-type UDP glycosyltransferase polypeptide, which have presently been discovered to confer the enzyme with increased activity for catalyzing the glycosylation of its intended substrate. This has the beneficial result of increased production of a steviol glycoside product and diminished production of undesired byproducts. Particularly, it has been discovered that expression of a variant UDP glycosyltransferase of the disclosure in a yeast cell genetically modified to produce one or more steviol glycosides augments the total yield and purity of the steviol glycoside relative to a counterpart yeast strain modified to synthesize the steviol glycoside but that expresses a wild-type UDP glycosyltransferase. The sections that follow describe, in further detail, the types of modifications that variant UDP glycosyltransferase polypeptides of the disclosure exhibit and how these polypeptides can be used to produce a desired steviol glycoside.

In a first aspect, the disclosure provides a variant UDP glycosyltransferase polypeptide including one or more amino acid substitutions relative to the amino acid sequence of SEQ ID NO: 1. The one or more amino acid substitutions may include an amino acid substitution at a residue selected from G4, R9, P65, V66, R94, V110, R187, D195, L201, S363, G385, R389, and D404.

In some embodiments, the one or more amino acid substitutions include an amino acid substitution at residue G4 of SEQ ID NO: 1. In some embodiments, the amino acid substitution at residue G4 of SEQ ID NO: 1 substitutes G4 with an amino acid including a polar, uncharged side chain at physiological pH. In some embodiments, the amino acid substitution at residue G4 of SEQ ID NO: 1 is a G4N substitution.

In some embodiments, the one or more amino acid substitutions include an amino acid substitution at residue R9 of SEQ ID NO: 1. In some embodiments, the amino acid substitution at residue R9 of SEQ ID NO: 1 substitutes R9 with an amino acid including a polar, uncharged side chain at physiological pH. In some embodiments, the amino acid substitution at residue R9 of SEQ ID NO: 1 is an R9S substitution.

In some embodiments, the one or more amino acid substitutions include an amino acid substitution at residue P65 of SEQ ID NO: 1. In some embodiments, the amino acid substitution at residue P65 of SEQ ID NO: 1 substitutes P65 with an amino acid including a polar, uncharged side chain at physiological pH. In some embodiments, the amino acid substitution at residue P65 of SEQ ID NO: 1 is a P65S substitution.

In some embodiments, the one or more amino acid substitutions include an amino acid substitution at residue V66 of SEQ ID NO: 1. In some embodiments, the amino acid substitution at residue V66 of SEQ ID NO: 1 substitutes V66 with an amino acid including a cationic side chain at physiological pH. In some embodiments, the amino acid substitution at residue V66 of SEQ ID NO: 1 is a V66R substitution. In some embodiments, the amino acid substitution at residue V66 of SEQ ID NO: 1 substitutes V66 with an amino acid including a hydrophobic, uncharged side chain at physiological pH. In some embodiments, the amino acid substitution at residue V66 of SEQ ID NO: 1 is a V66F substitution.

In some embodiments, the one or more amino acid substitutions include an amino acid substitution at residue R94 of SEQ ID NO: 1. In some embodiments, the amino acid substitution at residue R94 of SEQ ID NO: 1 substitutes R94 with an amino acid including a polar, uncharged side chain at physiological pH. In some embodiments, the amino acid substitution at residue R94 of SEQ ID NO: 1 is an R94N substitution.

In some embodiments, the one or more amino acid substitutions include an amino acid substitution at residue V110 of SEQ ID NO: 1. In some embodiments, the amino acid substitution at residue V110 of SEQ ID NO: 1 substitutes V110 with an amino acid including a polar, uncharged chain at physiological pH. In some embodiments, the amino acid substitution at residue V110 of SEQ ID NO: 1 is a V110S substitution.

In some embodiments, the one or more amino acid substitutions include an amino acid substitution at residue R187 of SEQ ID NO: 1. In some embodiments, the amino acid substitution at residue R187 of SEQ ID NO: 1 is an R187P substitution.

In some embodiments, the one or more amino acid substitutions include an amino acid substitution at residue D195 of SEQ ID NO: 1. In some embodiments, the amino acid substitution at residue D195 of SEQ ID NO: 1 substitutes D195 with an amino acid including a hydrophobic, uncharged side chain at physiological pH. In some embodiments, the amino acid substitution at residue D195 of SEQ ID NO: 1 is a D195A substitution.

In some embodiments, the one or more amino acid substitutions include an amino acid substitution at residue L201 of SEQ ID NO: 1. In some embodiments, the amino acid substitution at residue L201 of SEQ ID NO: 1 substitutes L201 with an amino acid including a polar, uncharged side chain at physiological pH. In some embodiments, the amino acid substitution at residue L201 of SEQ ID NO: 1 is an L201N substitution.

In some embodiments, the one or more amino acid substitutions include an amino acid substitution at residue S363 of SEQ ID NO: 1. In some embodiments, the amino acid substitution at residue S363 of SEQ ID NO: 1 substitutes S363 with an amino acid including a polar, uncharged side chain at physiological pH. In some embodiments, the amino acid substitution at residue S363 of SEQ ID NO: 1 is an S363N substitution.

In some embodiments, the one or more amino acid substitutions include an amino acid substitution at residue G385 of SEQ ID NO: 1. In some embodiments, the amino acid substitution at residue G385 of SEQ ID NO: 1 substitutes G385 with an amino acid including a cationic side chain at physiological pH. In some embodiments, the amino acid substitution at residue G385 of SEQ ID NO: 1 is a G385H substitution. In some embodiments, the amino acid substitution at residue G385 of SEQ ID NO: 1 substitutes G385 with an amino acid including a hydrophobic, uncharged side chain at physiological pH. In some embodiments, the amino acid substitution at residue G385 of SEQ ID NO: 1 is a G3851 substitution.

In some embodiments, the one or more amino acid substitutions include an amino acid substitution at residue R389 of SEQ ID NO: 1. In some embodiments, the amino acid substitution at residue R389 of SEQ ID NO: 1 substitutes R389 with an amino acid including a cationic side chain at physiological pH. In some embodiments, the amino acid substitution at residue R389 of SEQ ID NO: 1 is an R389H substitution. In some embodiments, the amino acid substitution at residue R389 of SEQ ID NO: 1 substitutes R389 with an amino acid including an anionic side chain at physiological pH. In some embodiments, the amino acid substitution at residue R389 of SEQ ID NO: 1 is an R389D substitution. In some embodiments, the amino acid substitution at residue R389 of SEQ ID NO: 1 substitutes R389 with an amino acid including a polar, uncharged side chain at physiological pH. In some embodiments, the amino acid substitution at residue R389 of SEQ ID NO: 1 is an R389N substitution. In some embodiments, the amino acid substitution at residue R389 of SEQ ID NO: 1 substitutes R389 with an amino acid including a hydrophobic, uncharged side chain at physiological pH. In some embodiments, the amino acid substitution at residue R389 of SEQ ID NO: 1 is an R389F substitution.

In some embodiments, the one or more amino acid substitutions include an amino acid substitution at residue D404 of SEQ ID NO: 1. In some embodiments, the amino acid substitution at residue D404 of SEQ ID NO: 1 substitutes D404 with an amino acid including a polar, uncharged chain at physiological pH. In some embodiments, the amino acid substitution at residue D404 of SEQ ID NO: 1 is a D404T substitution. In some embodiments, the amino acid substitution at residue D404 of SEQ ID NO: 1 is a D404S substitution.

In some embodiments, the one or more amino acid substitutions include P65S, V66F, V110S, R187P, D195A, L201N, G385H, R389D, and D404T relative to SEQ ID NO: 1. In some embodiments, the one or more amino acid substitutions include R9S, P65S, V110S, R187P, L201N, and R389D relative to SEQ ID NO: 1. In some embodiments, the one or more amino acid substitutions include P65S, V110S, R187P, L201N, G385H, R389D, and D404T relative to SEQ ID NO: 1. In some embodiments, the one or more amino acid substitutions include G4N, R94N, D195A, L201N, G385H, and R389D relative to SEQ ID NO: 1. In some embodiments, the one or more amino acid substitutions include G4N, R94N, R187P, D195A, L201N, R389D, and D404T relative to SEQ ID NO: 1. In some embodiments, the one or more amino acid substitutions include R94N, R187P, L201N, R389D, and D404T relative to SEQ ID NO: 1. In some embodiments, the one or more amino acid substitutions include G4N, V16F, R94N, V110S, L201N, and R389D relative to SEQ ID NO: 1. In some embodiments, the one or more amino acid substitutions include G4N, R9S, P65S, R187P, D195A, L201N, R389D, and D404T relative to SEQ ID NO: 1. In some embodiments, the one or more amino acid substitutions include R9S, R94N, D195A, L201N, G385H, R389D, and D404T relative to SEQ ID NO: 1. In some embodiments, the one or more amino acid substitutions include P65S, R94N, V110S, D195A, L201N, G385H, and R389D relative to SEQ ID NO: 1.

In some embodiments, the polypeptide has an amino acid sequence that is from about 85% to about 99.7% identical (e.g., 85.5%, 86%, 86.5%, 87%, 87.5%, 88%, 88.5%, 89%, 89.5%, 90%, 90.5%, 91%, 91.2%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1. In some embodiments, the polypeptide has an amino acid sequence that is from about 90% to about 99.7% identical (e.g., 90.5%, 91%, 91.2%, 92%, 92.5%, 93%, 93.5%, 94%, 94.5%, 95%, 95.5%, 96%, 96.5%, 97%, 97.5%, 98%, 98.5%, 99%, or 99.5% identical) to the amino acid sequence of SEQ ID NO: 1.

In some embodiments, the polypeptide has an amino acid sequence that differs from the amino acid sequence of SEQ ID NO: 1 only by way of the one or more amino acid substitutions or deletions and, optionally, one or more additional, conservative amino acid substitutions. In some embodiments, the polypeptide has an amino acid sequence that differs from the amino acid sequence of SEQ ID NO: 1 only by way of the one or more amino acid substitutions or deletions.

In some embodiments, the polypeptide has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NO: 2-30. In some embodiments, the polypeptide has an amino acid sequence that is at least 90% identical (e.g., at least 91%, 92%, 93%, 94%, 95%, 96%, 97%, 98%, or 99% identical) to the amino acid sequence of any one of SEQ ID NO: 2-30. In some embodiments, the polypeptide has an amino acid sequence that is at least 95% identical (e.g., at least 96%, 97%, 98%, or 99% identical) to the amino acid sequence of any one of SEQ ID NO: 2-30. In some embodiments, the polypeptide has the amino acid sequence of any one of SEQ ID NO: 2-30.

In some embodiments, the polypeptide catalyzes glycosylation at the 2′ position of the 13-O-glucose of a steviol glycoside, optionally wherein the polypeptide exhibits increased glycosylation activity at the 2′ position of the 13-O-glucose of a steviol glycoside as compared to a polypeptide having the amino acid sequence of SEQ ID NO: 1. In some embodiments, the polypeptide exhibits at least a 1.1-fold increase in glycosylation activity at the 2′ position of the 13-O-glucose of a steviol glycoside as compared to a polypeptide having the amino acid sequence of SEQ ID NO: 1. In some embodiments, the polypeptide exhibits between a 1.1-fold and 10-fold increase (e.g., a 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or a 10-fold increase) in glycosylation activity at the 2′ position of the 13-O-glucose of a steviol glycoside as compared to a polypeptide having the amino acid sequence of SEQ ID NO: 1.

In another aspect, the disclosure provides a nucleic acid encoding any one of the variant polypeptides described herein.

In another aspect, the disclosure provides a host cell including any one of the variant polypeptides described herein or the nucleic acid encoding any one of the variant polypeptides described herein. In some embodiments, the nucleic acid encoding the variant polypeptide is integrated into the genome of the cell. In some embodiments, the nucleic acid encoding the variant polypeptide is present within a plasmid.

In another aspect, disclosure provides a host cell capable of producing one or more steviol glycosides, wherein the host cell includes one or more heterologous nucleic acids that each, independently, encode a UDP glycosyltransferase. The UDP glycosyltransferase may have an amino acid sequence that is at least 90% identical (e.g., at least 91%, 92%, 93%, 94%, 95%, 96%, 97%, 98%, or 99% identical) to the amino acid sequence of any one of SEQ ID NO: 2-30. In some embodiments, the host cell includes one or more heterologous nucleic acids that each, independently, encode a UDP glycosyltransferase having an amino acid sequence that is at least 95% identical (e.g., at least 96%, 97%, 98%, or 99% identical) to the amino acid sequence of any one of SEQ ID NO: 2-30. In some embodiments, the UDP glycosyltransferase has the amino acid sequence of any one of SEQ ID NO: 2-30.

In some embodiments, the host cell includes one or more heterologous nucleic acids encoding a geranylgeranyl diphosphate synthase (GGPPS), a copalyl diphosphate synthase (CDPS), a kaurene synthase (KS), a kaurene oxidase (KO), a kaurene acid hydroxylase (KAH), a cytochrome P450 reductase (CPR), and one or more UDP glycosyltransferases.

In some embodiments, the host cell includes a heterologous nucleic acid encoding a GGPPS. In some embodiments, the GGPPS has an amino acid sequence that is at least 90% identical (e.g., at least 91%, 92%, 93%, 94%, 95%, 96%, 97%, 98%, or 99% identical) to the amino acid sequence of SEQ ID NO: 41. In some embodiments, the GGPPS has an amino acid sequence that is at least 95% identical (e.g., at least 96%, 97%, 98%, or 99% identical) to the amino acid sequence of SEQ ID NO: 41. In some embodiments, the GGPPS has the amino acid sequence of SEQ ID NO: 41.

In some embodiments, the host cell includes a heterologous nucleic acid encoding a CDPS. In some embodiments, the CDPS has an amino acid sequence that is at least 90% identical (e.g., at least 91%, 92%, 93%, 94%, 95%, 96%, 97%, 98%, or 99% identical) to the amino acid sequence of SEQ ID NO: 42. In some embodiments, the CDPS has an amino acid sequence that is at least 95% identical (e.g., at least 96%, 97%, 98%, or 99% identical) to the amino acid sequence of SEQ ID NO: 42. In some embodiments, the CDPS has the amino acid sequence of SEQ ID NO: 42.

In some embodiments, the host cell includes a heterologous nucleic acid encoding a KS. In some embodiments, the KS has an amino acid sequence that is at least 90% identical (e.g., at least 91%, 92%, 93%, 94%, 95%, 96%, 97%, 98%, or 99% identical) to the amino acid sequence of SEQ ID NO: 43. In some embodiments, the KS has an amino acid sequence that is at least 95% identical e.g., at least 96%, 97%, 98%, or 99% identical) to the amino acid sequence of SEQ ID NO: 43. In some embodiments, the KS has the amino acid sequence of SEQ ID NO: 43.

In some embodiments, the host cell includes a heterologous nucleic acid encoding a KO. In some embodiments, the KO has an amino acid sequence that is at least 90% identical (e.g., at least 91%, 92%, 93%, 94%, 95%, 96%, 97%, 98%, or 99% identical) to the amino acid sequence of SEQ ID NO: 44. In some embodiments, the KO has an amino acid sequence that is at least 95% identical (e.g., at least 96%, 97%, 98%, or 99% identical) to the amino acid sequence of SEQ ID NO: 44. In some embodiments, the KO has the amino acid sequence of SEQ ID NO: 44.

In some embodiments, the host cell includes a heterologous nucleic acid encoding a KAH. In some embodiments, the KAH has an amino acid sequence that is at least 90% identical (e.g., at least 91%, 92%, 93%, 94%, 95%, 96%, 97%, 98%, or 99% identical) to the amino acid sequence of SEQ ID NO: 46. In some embodiments, the KAH has an amino acid sequence that is at least 95% identical (e.g., at least 96%, 97%, 98%, or 99% identical) to the amino acid sequence of SEQ ID NO: 46. In some embodiments, the KAH has the amino acid sequence of SEQ ID NO: 46.

In some embodiments, the host cell includes a heterologous nucleic acid encoding a CPR. In some embodiments, the CPR has an amino acid sequence that is at least 90% identical (e.g., at least 91%, 92%, 93%, 94%, 95%, 96%, 97%, 98%, or 99% identical) to the amino acid sequence of SEQ ID NO: 45. In some embodiments, the CPR has an amino acid sequence that is at least 95% identical (e.g., at least 96%, 97%, 98%, or 99% identical) to the amino acid sequence of SEQ ID NO: 45. In some embodiments, the CPR has the amino acid sequence of SEQ ID NO: 45.

In some embodiments, the host cell includes one or more heterologous nucleic acids encoding one or more additional UDP glycosyltransferases. In some embodiments, the one or more additional UDP glycosyltransferases are selected from a UGT74G1, a UGT85C2, a UGT40087, and a UGT76G1.

In some embodiments, the host cell includes a heterologous nucleic acid encoding a UGT74G1. In some embodiments, the UGT74G1 has an amino acid sequence that is at least 90% identical (e.g., at least 91%, 92%, 93%, 94%, 95%, 96%, 97%, 98%, or 99% identical) to the amino acid sequence of SEQ ID NO: 37. In some embodiments, the UGT74G1 has an amino acid sequence that is at least 95% identical (e.g., at least 96%, 97%, 98%, or 99% identical) to the amino acid sequence of SEQ ID NO: 37. In some embodiments, the UGT74G1 has the amino acid sequence of SEQ ID NO: 37.

In some embodiments, the host cell includes a heterologous nucleic acid encoding a UGT85C2. In some embodiments, the UGT85C2 has an amino acid sequence that is at least 90% identical (e.g., at least 91%, 92%, 93%, 94%, 95%, 96%, 97%, 98%, or 99% identical) to the amino acid sequence of SEQ ID NO: 36. In some embodiments, the UGT85C2 has an amino acid sequence that is at least 95% identical (e.g., at least 96%, 97%, 98%, or 99% identical) to the amino acid sequence of SEQ ID NO: 36. In some embodiments, the UGT85C2 has the amino acid sequence of SEQ ID NO: 36.

In some embodiments, the host cell includes a heterologous nucleic acid encoding a UGT40087. In some embodiments, the UGT40087 has an amino acid sequence that is at least 90% identical (e.g., at least 91%, 92%, 93%, 94%, 95%, 96%, 97%, 98%, or 99% identical) to the amino acid sequence of SEQ ID NO: 40. In some embodiments, the UGT40087 has an amino acid sequence that is at least 95% identical (e.g., at least 96%, 97%, 98%, or 99% identical) to the amino acid sequence of SEQ ID NO: 40. In some embodiments, the UGT40087 has the amino acid sequence of SEQ ID NO: 40.

In some embodiments, the host cell includes a heterologous nucleic acid encoding a UGT76G1. In some embodiments, the UGT76G1 has an amino acid sequence that is at least 90% identical (e.g., at least 91%, 92%, 93%, 94%, 95%, 96%, 97%, 98%, or 99% identical) to the amino acid sequence of SEQ ID NO: 39. In some embodiments, the UGT76G1 has an amino acid sequence that is at least 95% identical (e.g., at least 96%, 97%, 98%, or 99% identical) to the amino acid sequence of SEQ ID NO: 39. In some embodiments, the UGT76G1 has the amino acid sequence of SEQ ID NO: 39.

In some embodiments, the one or more heterologous nucleic acids are present within one or more plasmids in the host cell. In some embodiments, the one or more heterologous nucleic acids are integrated into the genome of the host cell.

In some embodiments, the one or more steviol glycosides are selected from RebA, RebB, RebD, RebE, and RebM. In some embodiments, the one or more steviol glycosides include RebM.

In some embodiments, the host cell is selected from a bacterial cell, a yeast cell, an algal cell, an insect cell, and a plant cell. In some embodiments, the host cell is a yeast cell. In some embodiments, the yeast cell is Saccharomyces cerevisiae.

In another aspect, the disclosure provides a method for producing one or more steviol glycosides. In some embodiments, the method includes culturing a population of any one of the host cells described herein in a medium with a carbon source under conditions suitable for making one or more steviol glycosides, thereby yielding a culture broth. The method may further include recovering the one or more steviol glycosides from the culture broth. In some embodiments, the one or more steviol glycosides are selected from RebA, RebB, RebD, RebE, and RebM. In some embodiments, the one or more steviol glycosides include RebM.

In another aspect, the disclosure provides a fermentation composition including a population of any one of the host cells described herein, and one or more steviol glycosides produced by the host cell. In some embodiments, the one or more steviol glycosides are selected from RebA, RebB, RebD, RebE, and RebM. In some embodiments, the one or more steviol glycosides include RebM.

In another aspect, the disclosure provides a composition including a steviol glycoside produced by any one of the methods described herein. In some embodiments, the steviol glycoside is selected from RebA, RebB, RebD, RebE, and RebM. In some embodiments, the steviol glycoside is RebM.

### Definitions

As used in this specification and the appended claims, the singular forms “a,” “an,” and “the” include plural reference unless the context clearly dictates otherwise.

As used herein, the term “about” is used herein to mean a value that is ±10% of the recited value.

As used herein, the term “capable of producing” refers to a host cell that is genetically modified to express the enzyme(s) necessary for the production of a given compound in accordance with a biochemical pathway that produces the compound. For example, a host cell (e.g., a yeast cell) that is “capable of producing” a steviol glycoside is one that expresses the enzymes necessary for production of the steviol glycoside according to the biosynthetic pathway for the steviol glycoside of interest.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein in the context of a gene, the term “express” refers to any one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a cell, tissue sample, or subject can manifest, for example, as: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art).

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that includes a polynucleotide sequence encoding a polypeptide for use in the invention operably linked to a promoter, e.g., its native promoter, where the expression cassette is introduced into a heterologous microorganism. In some embodiments, an expression cassette includes a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “fermentation composition” refers to a composition which comprises genetically modified host cells and products or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro-RNA.

A “genetic pathway” or “biosynthetic pathway” as used herein refers to a set of at least two different coding sequences, where the coding sequences encode enzymes that catalyze different parts of a synthetic pathway to form a desired product (e.g., a steviol glycoside). In a genetic pathway, a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus.

The term “host cell” as used in the context of this disclosure refers to a microorganism, such as yeast, and includes an individual cell or cell culture including a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “introducing” in the context of a nucleic acid or protein in a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The term also encompasses integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “medium” refers to culture medium and/or fermentation medium.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion.

As used herein, the terms “native” or “endogenous” with reference to molecules, and in particular polypeptides and polynucleotides, indicate molecules that are expressed in the organism in which they originated or are found in nature. It is understood that expression of native polypeptides or polynucleotides may be modified in recombinant organisms.

As used herein, the term “parent cell” refers to a cell that has an identical genetic background as a genetically modified host cell disclosed herein except that it does not comprise one or more particular genetic modifications engineered into the modified host cell, for example, heterologous expression of an enzyme of a steviol glycoside pathway, such as heterologous expression of a geranylgeranyl diphosphate synthase, heterologous expression of a copalyl diphosphate synthase, heterologous expression of a kaurene synthase, heterologous expression of a kaurene oxidase, heterologous expression of a kaurenoic acid hydroxylase, heterologous expression of a cytochrome P450 reductase, and/or heterologous expression of a UDP-glycosyltransferase, such as EUGT11, UGT74G1, UGT76G1, UGT85C2, UGT91 D, and UGT40087, or a variant thereof.

As used herein, the term “operably linked” refers to a functional linkage between nucleic acid sequences such that the sequences encode a desired function. For example, a coding sequence for a gene of interest is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in a host cell.

As used herein, the term “overexpression” refers to a process of genetically modifying a host cell to express a polypeptide or RNA molecule in an amount that exceeds the amount of the polypeptide or RNA that would be observed in a host cell of the same species but that has not been subject to the genetic modification. Exemplary methods of overexpressing a polypeptide or RNA molecule of the disclosure include expressing the polypeptide or RNA molecule in a host cell under the control of a highly active transcription regulatory element, such as a promoter or enhancer that fosters expression of the polypeptide or RNA at levels that exceed wild-type expression levels observed in an unmodified host cell of the same species.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present disclosure will generally contain phosphodiester bonds, although in some cases, nucleic acid analogs may be used that may have alternate backbones, including, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA, or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of steviol glycoside produced by a genetically modified host cell provided herein. In some embodiments, production is expressed as a yield of steviol glycoside by the host cell. In other embodiments, production is expressed as the productivity of the host cell in producing the steviol glycoside.

As used herein, the term “productivity” refers to production of steviol glycoside by a host cell, expressed as the amount of steviol glycoside produced (by weight) per amount of fermentation broth in which the host cell is cultured (by volume) over time (per hour).

As used herein, the term “promoter” refers to a synthetic or naturally derived nucleic acid that is capable of activating, increasing, or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

As used herein, the term “rebaudioside M” or “RebM” refers to a steviol glycoside having the following structure:

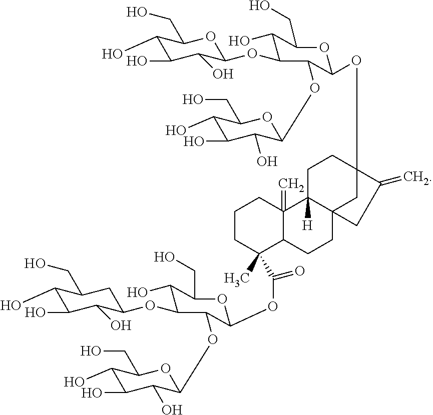

As used herein, the term “signal sequence” or “N-terminal signal sequence” refers to a short peptide (e.g., 5-50 amino acids in length) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the endoplasmic reticulum. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). An N-terminal signal sequence may be replaced with a corresponding amino acid sequence encoding a heterologous N-terminal signal sequence (e.g., an N-terminal signal sequence from plant p450 polypeptide)

As used herein, the term “steviol” refers to the compound steviol, including any stereoisomer of steviol. In preferred embodiments, the term refers to the compound having the following structure:

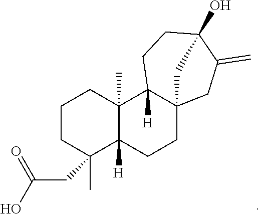

As used herein, the term “steviol glycoside” refers to a glycoside of steviol including but not limited to 19-glycoside, steviolmonoside, steviolbioside, rubusoside, dulcoside B, dulcoside A, rebaudioside A (RebA), rebaudioside B (RebB), rebaudioside C (RebC), rebaudioside D (RebD), rebaudioside E (RebE), rebaudioside F (RebF), rebaudioside G (RebG), rebaudioside H (RebH), rebaudioside I (RebI), rebaudioside J (RebJ), rebaudioside K (RebK), rebaudioside L (RebL), rebaudioside M (RebM), rebaudioside N (RebN), rebaudioside O (RebO), rebaudioside D2, and rebaudioside M2.

Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using a sequence comparison algorithm or by manual alignment and visual inspection as described above. Optionally, the identity exists over a region that is at least about 50 nucleotides (or 20 amino acids) in length, or more preferably over a region that is 100 to 500 or 1000 or more nucleotides (or 50, 100, or 200 or more amino acids) in length.

Nucleic acid or protein sequences that are substantially identical to a reference sequence include “conservatively modified variants.” With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu, and lie; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. The following six groups each contain amino acids that further provide illustrative conservative substitutions for one another. 1) Ala, Ser, Thr; 2) Asp, Glu; 3) Asn, Gln; 4) Arg, Lys; 5) Ile, Leu, Met, Val; and 6) Phe, Try, and Trp (see, e.g., Creighton, Proteins: Structures and Molecular Principles. 1984, New York: W.H. Freeman).

Accordingly, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in Table 1, below.

TABLE 1
Representative physicochemical properties
of naturally occurring amino acids

| | 3 | 1 | Side- | Electrostatic | |
|---|---|---|---|---|---|
| | Letter | Letter | chain | character at | Steric |
| Amino Acid | Code | Code | Polarity | pH = 7.4 | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

As used herein, the term “transformation” refers to a genetic alteration of a host cell resulting from the introduction of exogenous genetic material, e.g., nucleic acids, into the host cell.

As used herein, the term “variant” refers to molecules, and in particular polypeptides and polynucleotides, that differ from a specifically recited “reference” molecule in either structure or sequence. In preferred embodiments, the reference is a wild-type molecule. With respect to polypeptides and polynucleotides, variants refer to substitutions, additions, or deletions of the amino acid or nucleotide sequences respectively.

As used herein, the term “yield” refers to production of a steviol glycoside by a host cell, expressed as the amount of steviol glycoside produced per amount of carbon source consumed by the host cell, by weight.

### BRIEF DESCRIPTION OF THE DRAWINGS

FIG. 1 is a schematic showing an enzymatic pathway from the native yeast metabolite farnesyl pyrophosphate (FPP) to RebM.

FIG. 2 is a schematic of the landing pad DNA construct used to insert UGT91 D homologous genes into RebM strains. The landing pad consists of 500 bp of locus-targeting DNA sequences on either end of the construct to the genomic region upstream and downstream of the yeast locus of choice. The locus is chosen so that insertion of the landing pad does not delete any gene. Internally the landing pad contains a GAL promoter followed by a recognition site for the F-CphI endonuclease and the yeast terminator. Endonuclease F-CphI cuts the recognition sequence creating a double strand break at the landing pad thus facilitating homologous recombination of the UGT91 D_like3 DNA variants at the site.

FIG. 3 is a graph of RebM measured in μM in whole cell broth relative to the Sr.UGT91 D_like3 control. Yeast strains with different UGT genes expressed under pGAL1 were grown in microtiter plates. Also shown are the data for the parent strain that does not contain any Sr.UGT91 D_like3 homolog. Dark vertical lines represent 95% confidence interval of the mean (N=16).

FIG. 4 is a graph of the combined titers of glycosylated products with three, four, and five glucose moieties measured in μM in whole cell broth relative to Sr.UGT91 D_like3 control. In yeast host containing only UGT74G1 and UGT85C2 exogenous glycosyltransferases (thus producing only singly and doubly glycosylated compounds), different UGT genes were expressed under pGAL1 and resulting strains were grown in microtiter plates. Also shown are the data for the parent strain that does not contain any Sr.UGT91 D_like3 homolog. Dark vertical lines represent 95% confidence interval of the mean (N=8).

FIG. 5 is a graph depicting the composition of advanced glycosylated products stevioside, RebE, and [Steviol+5 Glucose (Glc)]′, as molar fractions, produced by yeast strains containing UGT74G1, UGT85C2, and different UGT genes grown in microtiter plates. These are same strains and cultivations as in FIG. 4.

FIG. 6 depicts the proposed reactions catalyzed by seven UGT91 D glycosyltransferases tested when only two other glycosyltransferases are present, UGT74G1 and UGT85C2 (partial pathway). In the presence of UGT76G1, RebE would be converted to RebM. In the absence of UGT76G1, RebE is glycosylated to undesirable side product, [Steviol+5 Glc]′. The structure of [Steviol+5 Glc]′ depicted here is tentative.

### DETAILED DESCRIPTION

The present disclosure features variant uridine-5′-diphosphate (UDP) glycosyltransferase polypeptides, nucleic acids encoding the same, host cells capable of producing one or more steviol glycosides, and methods of producing one or more steviol glycosides in a host cell, such as a yeast cell. The variant UDP glycosyltransferases described herein contain modifications, such as amino acid substitutions, which have presently been discovered to impart the polypeptide with enhanced glycosyltransferase activity of glycosylating the 2′ position of the 13-O-glucose of a steviol glycoside. This increased activity gives rise to the ability to increase production of a target steviol glycoside with greater purity and overall yield relative to methods using a wild-type UDP glycosyltransferase enzyme.

For example, expression of a variant UDP glycosyltransferase polypeptide of the disclosure in a yeast strain capable of producing a desired steviol glycoside may result in enhanced purity and improved yield of the target steviol glycoside in comparison to a counterpart yeast strain that expresses a wild-type UDP glycosyltransferase.

The following sections provide a detailed description of the amino acid modifications (e.g., substitutions) that have been discovered to engender the enhanced activity described above, and detail how these variant UDP glycosyltransferase polypeptides can be utilized to generate a desired steviol glycoside.

### Uridine-5′-Diphosphate Glycosyltransferase Polypeptides

The variant UDP glycosyltransferase polypeptides of the disclosure can be used to produce one or more steviol glycosides, including, without limitation, RebM, among others described herein. The UDP glycosyltransferase modifications described herein give rise to beneficial biosynthetic properties, as these modifications promote heightened yield of a target steviol glycoside product in comparison to a host cell which expresses the corresponding wild-type UDP glycosyltransferase.

In some embodiments, a variant UDP glycosyltransferase polypeptide contains one or more amino acid substitutions relative to the amino acid sequence of SEQ ID NO: 1. The amino acid substitution may occur, for example, at a residue selected from G4, R9, P65, V66, R94, V110, R187, D195, L201, S363, G385, R389, and D404 of SEQ ID NO: 1.

In some embodiments, the variant polypeptide includes an amino acid substitution at residue G4 of SEQ ID NO: 1. For example, the amino acid substitution at residue G4 of SEQ ID NO: 1 may substitute G4 with an amino acid including a polar, uncharged side chain at physiological pH. In some embodiments, the amino acid substitution at residue G4 of SEQ ID NO: 1 is a G4N substitution.

In some embodiments, the variant polypeptide includes an amino acid substitution at residue R9 of SEQ ID NO: 1. For example, the amino acid substitution at residue R9 of SEQ ID NO: 1 may substitute R9 with an amino acid including a polar, uncharged side chain at physiological pH. In some embodiments, the amino acid substitution at residue R9 of SEQ ID NO: 1 is an R9S substitution.

In some embodiments, the variant polypeptide includes an amino acid substitution at residue P65 of SEQ ID NO: 1. For example, the amino acid substitution at residue P65 of SEQ ID NO: 1 may substitute P65 with an amino acid including a polar, uncharged side chain at physiological pH. In some embodiments, the amino acid substitution at residue P65 of SEQ ID NO: 1 is a P65S substitution.

In some embodiments, the variant polypeptide includes an amino acid substitution at residue V66 of SEQ ID NO: 1. For example, the amino acid substitution at residue V66 of SEQ ID NO: 1 may substitute V66 with an amino acid including a cationic side chain at physiological pH. In some embodiments, the amino acid substitution at residue V66 of SEQ ID NO: 1 is a V66R substitution. In some embodiments, the amino acid substitution at residue V66 of SEQ ID NO: 1 may substitute V66 with an amino acid comprising a hydrophobic, uncharged side chain at physiological pH. In some embodiments, the amino acid substitution at residue V66 of SEQ ID NO: 1 is a V66F substitution.

In some embodiments, the variant polypeptide of includes an amino acid substitution at residue R94 of SEQ ID NO: 1. For example, the amino acid substitution at residue R94 of SEQ ID NO: 1 may substitute R94 with an amino acid including a polar, uncharged side chain at physiological pH. In some embodiments, the amino acid substitution at residue R94 of SEQ ID NO: 1 is an R94N substitution.

In some embodiments, the variant polypeptide includes an amino acid substitution at residue V110 of SEQ ID NO: 1. For example, the amino acid substitution at residue V110 of SEQ ID NO: 1 may substitute V110 with an amino acid including a polar, uncharged chain at physiological pH. In some embodiments, the amino acid substitution at residue V110 of SEQ ID NO: 1 is a V110S substitution.

In some embodiments, the variant polypeptide includes an amino acid substitution at residue R187 of SEQ ID NO: 1. In some embodiments, the amino acid substitution at residue R187 of SEQ ID NO: 1 is an R187P substitution.

In some embodiments, the variant polypeptide includes an amino acid substitution at residue D195 of SEQ ID NO: 1. For example, the amino acid substitution at residue D195 of SEQ ID NO: 1 may substitute D195 with an amino acid including a hydrophobic, uncharged side chain at physiological pH. In some embodiments, the amino acid substitution at residue D195 of SEQ ID NO: 1 is a D195A substitution.

In some embodiments, the variant polypeptide includes an amino acid substitution at residue L201 of SEQ ID NO: 1. For example, the amino acid substitution at residue L201 of SEQ ID NO: 1 may substitute L201 with an amino acid including a polar, uncharged side chain at physiological pH. In some embodiments, the amino acid substitution at residue L201 of SEQ ID NO: 1 is an L201N substitution.

In some embodiments, the variant polypeptide includes an amino acid substitution at residue S363 of SEQ ID NO: 1. For example, the amino acid substitution at residue S363 of SEQ ID NO: 1 may substitute S363 with an amino acid including a polar, uncharged side chain at physiological pH. In some embodiments, the amino acid substitution at residue S363 of SEQ ID NO: 1 is an S363N substitution.

In some embodiments, the variant polypeptide includes an amino acid substitution at residue G385 of SEQ ID NO: 1. For example, the amino acid substitution at residue G385 of SEQ ID NO: 1 may substitute G385 with an amino acid including a cationic side chain at physiological pH. In some embodiments, the amino acid substitution at residue G385 of SEQ ID NO: 1 is a G385H substitution. In some embodiments, the amino acid substitution at residue G385 of SEQ ID NO: 1 may substitute G385 with an amino acid including a hydrophobic, uncharged side chain at physiological pH. In some embodiments, the amino acid substitution at residue G385 of SEQ ID NO: 1 is a G3851 substitution.

In some embodiments, the variant polypeptide includes an amino acid substitution at residue R389 of SEQ ID NO: 1. For example, the amino acid substitution at residue R389 of SEQ ID NO: 1 may substitute R389 with an amino acid including a cationic side chain at physiological pH. In some embodiments, the amino acid substitution at residue R389 of SEQ ID NO: 1 is an R389H substitution. In some embodiments, the amino acid substitution at residue R389 of SEQ ID NO: 1 may substitute R389 with an amino acid including an anionic side chain at physiological pH. In some embodiments, the amino acid substitution at residue R389 of SEQ ID NO: 1 is an R389D substitution. In some embodiments, the amino acid substitution at residue R389 of SEQ ID NO: 1 may substitute R389 with an amino acid including a polar, uncharged side chain at physiological pH. In some embodiments, the amino acid substitution at residue R389 of SEQ ID NO: 1 is an R389N substitution. In some embodiments, the amino acid substitution at residue R389 of SEQ ID NO: 1 may substitute R389 with an amino acid including a hydrophobic, uncharged side chain at physiological pH. In some embodiments, the amino acid substitution at residue R389 of SEQ ID NO: 1 is an R389F substitution.

In some embodiments, the variant polypeptide includes an amino acid substitution at residue D404 of SEQ ID NO: 1. For example, the amino acid substitution at residue D404 of SEQ ID NO: 1 may substitute D404 with an amino acid including a polar, uncharged chain at physiological pH. In some embodiments, the amino acid substitution at residue D404 of SEQ ID NO: 1 is a D404T substitution. In some embodiments, the amino acid substitution at residue D404 of SEQ ID NO: 1 is a D404S substitution.

In some embodiments, the variant polypeptide includes one or more amino acid substitutions selected from P65S, V66F, V110S, R187P, D195A, L201N, G385H, R389D, and D404T relative to SEQ ID NO: 1. For example, the variant polypeptide may include the amino acid substitutions P65S, V66F, V110S, R187P, D195A, L201N, G385H, R389D, and D404T relative to SEQ ID NO: 1.

In some embodiments, the variant polypeptide includes the one or more amino acid substitutions selected from R9S, P65S, V110S, R187P, L201N, and R389D relative to SEQ ID NO: 1. For example, the variant polypeptide may include the amino acid substitutions R9S, P65S, V110S, R187P, L201N, and R389D relative to SEQ ID NO: 1.

In some embodiments, the variant polypeptide includes the one or more amino acid substitutions selected from P65S, V110S, R187P, L201N, G385H, R389D, and D404T relative to SEQ ID NO: 1. For example, the variant polypeptide may include the amino acid substitutions selected from P65S, V110S, R187P, L201N, G385H, R389D, and D404T relative to SEQ ID NO: 1.

In some embodiments, the variant polypeptide includes the one or more amino acid substitutions selected from G4N, R94N, D195A, L201N, G385H, and R389D relative to SEQ ID NO: 1. For example, the variant polypeptide may include the amino acid substitutions G4N, R94N, D195A, L201N, G385H, and R389D relative to SEQ ID NO: 1.

In some embodiments, the variant polypeptide includes the one or more amino acid substitutions selected from G4N, R94N, R187P, D195A, L201N, R389D, and D404T relative to SEQ ID NO: 1. For example, the variant polypeptide may include the amino acid substitutions G4N, R94N, R187P, D195A, L201N, R389D, and D404T relative to SEQ ID NO: 1.

In some embodiments, the variant polypeptide includes the one or more amino acid substitutions selected from R94N, R187P, L201N, R389D, and D404T relative to SEQ ID NO: 1. For example, the variant polypeptide may include the amino acid substitutions R94N, R187P, L201N, R389D, and D404T relative to SEQ ID NO: 1.

In some embodiments, the variant polypeptide includes the one or more amino acid substitutions selected from G4N, V16F, R94N, V110S, L201N, and R389D relative to SEQ ID NO: 1. For example, the variant polypeptide may include the amino acid substitutions G4N, V16F, R94N, V110S, L201N, and R389D relative to SEQ ID NO: 1

In some embodiments, the variant polypeptide includes the one or more amino acid substitutions selected from G4N, R9S, P65S, R187P, D195A, L201N, R389D, and D404T relative to SEQ ID NO: 1. For example, the variant polypeptide may include the amino acid substitutions G4N, R9S, P65S, R187P, D195A, L201N, R389D, and D404T relative to SEQ ID NO: 1.

In some embodiments, the variant polypeptide includes the one or more amino acid substitutions selected from R9S, R94N, D195A, L201N, G385H, R389D, and D404T relative to SEQ ID NO: 1. For example, the variant polypeptide may include the amino acid substitutions R9S, R94N, D195A, L201N, G385H, R389D, and D404T relative to SEQ ID NO: 1.

In some embodiments, the variant polypeptide includes the one or more amino acid substitutions selected from P65S, R94N, V110S, D195A, L201N, G385H, and R389D relative to SEQ ID NO: 1. For example, the variant polypeptide may include the amino acid substitutions P65S, R94N, V110S, D195A, L201N, G385H, and R389D relative to SEQ ID NO: 1.

Illustrative variant UDP glycosyltransferase polypeptide sequences that may be used in conjunction with the compositions and methods described herein include, without limitation, SEQ ID NO: 2-30, as well as functional variants thereof.

In some embodiments, polypeptide has an amino acid sequence that is from about 85% to about 99.7% (e.g., 86%, 87%, 88%, 89%, 90%, 91%, 92%, 93%, 94%, 95%, 96%, 97%, 98%, 99%, or 99.5%) identical to the amino acid sequence of SEQ ID NO: 1. In some embodiments, the polypeptide has an amino acid sequence that is from about 90% to about 99.7% (e.g., 91%, 92%, 93%, 94%, 95%, 96%, 97%, 98%, 99%, or 99.5%) identical to the amino acid sequence of SEQ ID NO: 1. In some embodiments, the polypeptide has an amino acid sequence that differs from the amino acid sequence of SEQ ID NO: 1 only by way of the one or more amino acid substitutions or deletions and, optionally, one or more additional, conservative amino acid substitutions. In some embodiments, the polypeptide has an amino acid sequence that differs from the amino acid sequence of SEQ ID NO: 1 only by way of the one or more amino acid substitutions or deletions.

In some embodiments, the polypeptide has an amino acid sequence that is at least 85% (e.g., at least 86%, 87%, 88%, 89%, 90%, 91%, 92%, 93%, 94%, 95%, 96%, 97%, 98%, or 99%) identical to the amino acid sequence of any one of SEQ ID NO: 2-30. In some embodiments, the polypeptide has an amino acid sequence that is at least 90% (e.g., at least 91%, 92%, 93%, 94%, 95%, 96%, 97%, 98%, or 99%) identical to the amino acid sequence of any one of SEQ ID NO: 2-30. In some embodiments, the polypeptide has an amino acid sequence that is at least 95% (e.g., at least 95%, 96%, 97%, 98%, or 99%) identical to the amino acid sequence of any one of SEQ ID NO: 2-30. In some embodiments, the polypeptide has the amino acid sequence of any one of SEQ ID NO: 2-30.

The variant polypeptide may catalyze glycosylation at the 2′ position of the 13-O-glucose of a steviol glycoside. In some embodiments, the polypeptide exhibits increased glycosylation activity at the 2′ position of the 13-O-glucose of a steviol glycoside as compared to a polypeptide having the amino acid sequence of SEQ ID NO: 1. For example, the polypeptide may exhibit at least a 1.1-fold increase in glycosylation activity at the 2′ position of the 13-O-glucose of a steviol glycoside as compared to a polypeptide having the amino acid sequence of SEQ ID NO: 1. In some embodiments, the polypeptide exhibits between a 1.1-fold and 10-fold increase (e.g., a 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, or a 10-fold increase) in glycosylation activity at the 2′ position of the 13-O-glucose of a steviol glycoside as compared to a polypeptide having the amino acid sequence of SEQ ID NO: 1.

### Host Cells Genetically Modified to Produce Steviol Glycosides

Provided herein are host cells capable of producing one or more steviol glycosides including RebA, RebB, RebD, RebE, or RebM. The host cells described herein may express a variant UDP glycosyl transferase polypeptide, e.g., any one of SEQ ID NO: 2-30 or another UDP glycosyltransferase polypeptide having an amino acid substitution and/or deletion described herein.

The host cells capable of producing one or more steviol glycosides may encode on or more enzymes of the steviol glycoside biosynthesis pathway. In some embodiments, the steviol glycoside biosynthesis pathway is activated in the genetically modified host cells by engineering the cells to express polynucleotides encoding enzymes capable of catalyzing the biosynthesis of steviol glycosides.

In some embodiments, the genetically modified host cells contain one or more heterologous polynucleotides encoding a geranylgeranyl diphosphate synthase (GGPPS), a copalyl diphosphate synthase (CDPS), a kaurene synthase (KS), a kaurene oxidase (KO), a kaurene acid hydroxylase (KAH), a cytochrome P450 reductase (CPR), and/or one or more additional UDP-glycosyltransferases, such as UGT74G1, UGT76G1, UGT85C2, UGT91 D, EUGT11, and/or UGT40087. In some embodiments, the genetically modified host cells contain one or more heterologous polynucleotides encoding a variant GGPPS, CDPS, KS, KO, KAH, CPR, UDP-glycosyltransferase, UGT74G1, UGT76G1, UGT85C2, UGT91 D, EUGT11, and/or UGT40087. In certain embodiments, the variant enzyme may have from 1 up to 20 (e.g., 1, 2, 3, 4, 5, 6, 7, 8, 9, 10, 11, 12, 13 13, 15, 16, 17, 18, 19, or 20) amino acid substitutions relative to a reference enzyme. In certain embodiments, the coding sequence of the polynucleotide is codon optimized for the particular host cell.

### Geranylgeranyl Diphosphate Synthase

GGPPS (EC 2.5.1.29) catalyzes the conversion of farnesyl pyrophosphate into geranylgeranyl diphosphate. Examples of GGPPS include those of Stevia rebaudiana (accession no. ABD92926), Gibberella fujikuroi (accession no. CAA75568), Mus musculus (accession no. AAH69913), Thalassiosira pseudonana (accession no. XP_002288339), Streptomyces clavuligerus (accession no. ZP-05004570), Sulfulobus acidocaldarius (accession no. BAA43200), Synechococcus sp. (accession no. ABC98596), Arabidopsis thaliana (accession no. MP_195399), and Blakeslea trispora (accession no. AFC92798.1), and those described in U.S. Pat. No. 9,631,215.

In some embodiments, the host cell includes a heterologous nucleic acid encoding a GGPPS. In some embodiments, the GGPPS has an amino acid sequence that is at least 90% (e.g., at least 90%, 91%, 92%, 93%, 94%, 95%, 96%, 97%, 98%, or 99%) identical to the amino acid sequence of SEQ ID NO: 41. In some embodiments, the GGPPS has an amino acid sequence that is at least 95% (e.g., at least 95%, 96%, 97%, 98%, or 99%) identical to the amino acid sequence of SEQ ID NO: 41. In some embodiments, the GGPPS has the amino acid sequence of SEQ ID NO: 41.

### Copalyl Diphosphate Synthase

CDPS (EC 5.5.1.13) catalyzes the conversion of geranylgeranyl diphosphate into copalyl diphosphate. Examples of copalyl diphosphate synthases include those from Stevia rebaudiana (accession no. AAB87091), Streptomyces clavuligerus (accession no. EDY51667), Bradyrhizobioum japonicum (accession no. AAC28895.1), Zea mays (accession no. AY562490), Arabidopsis thaliana (accession no. NM_116512), and Oryza sativa (accession no. Q5MQ85.1), and those described in U.S. Pat. No. 9,631,215.

In some embodiments, the host cell includes a heterologous nucleic acid encoding a CDPS. In some embodiments, the CDPS has an amino acid sequence that is at least 90% (e.g., at least 90%, 91%, 92%, 93%, 94%, 95%, 96%, 97%, 98%, or 99%) identical to the amino acid sequence of SEQ ID NO: 42. In some embodiments, the CDPS has an amino acid sequence that is at least 95% (e.g., at least 95%, 96%, 97%, 98%, or 99%) identical to the amino acid sequence of SEQ ID NO: 42. In some embodiments, the CDPS has the amino acid sequence of SEQ ID NO: 42.

### Kaurene Synthase

KS (EC 4.2.3.19) catalyzes the conversion of copalyl diphosphate into kaurene and diphosphate. Examples of enzymes include those of Bradyrhizobium japonicum (accession no. AAC28895.1), Arabidopsis thaliana (accession no. Q9SAK2), and Picea glauca (accession no. ADB55711.1), and those described in U.S. Pat. No. 9,631,215.

In some embodiments, the host cell includes a heterologous nucleic acid encoding a KS. In some embodiments, the KS has an amino acid sequence that is at least 90% (e.g., at least 90%, 91%, 92%, 93%, 94%, 95%, 96%, 97%, 98%, or 99%) identical to the amino acid sequence of SEQ ID NO: 43. In some embodiments, the KS has an amino acid sequence that is at least 95% (e.g., at least 95%, 96%, 97%, 98%, or 99%) identical to the amino acid sequence of SEQ ID NO: 43. In some embodiments, the KS has the amino acid sequence of SEQ ID NO: 43.

### Bifunctional Copalyl Diphosphate Synthase and Kaurene Synthase

CDPS-KS bifunctional enzymes (EC 5.5.1.13 and EC 4.2.3.19) may also be used in the host cells of the invention. Examples include those of Phomopsis amygdali (accession no. BAG30962), Phaeosphaeria sp. (accession no. 013284), Physcomitrella patens (accession no. BAF61135), and Gibberella fujikuroi (accession no. Q9UVY5.1), and those described in U.S. Patent Application Publication Nos. 2014/032928 A1, 2014/0357588 A1, 2015/0159188, and WO 2016/038095.

### Kaurene Oxidase

KO (EC 1.14.13.88) catalyzes the conversion of kaurene into kaurenoic acid. Illustrative examples of enzymes include those of Oryza sativa (accession no. Q5Z5R4), Gibberella fujikuroi (accession no. 094142), Arabidopsis thaliana (accession no. Q93ZB2), Stevia rebaudiana (accession no. AAQ63464.1), and Pisum sativum (Uniprot no. Q6XAF4), and those described in U.S. Patent Application Publication Nos. 2014/0329281 A1, 2014/0357588 A1, 2015/0159188, and WO 2016/038095.

In some embodiments, the host cell includes a heterologous nucleic acid encoding a KO. In some embodiments, the KO has an amino acid sequence that is at least 90% (e.g., at least 90%, 91%, 92%, 93%, 94%, 95%, 96%, 97%, 98%, or 99%) identical to the amino acid sequence of SEQ ID NO: 44. In some embodiments, the KO has an amino acid sequence that is at least 95% (e.g., at least 95%, 96%, 97%, 98%, or 99%) identical to the amino acid sequence of SEQ ID NO: 44. In some embodiments, the KO has the amino acid sequence of SEQ ID NO: 44.

### Kaurenoic Acid Hydroxylase

KAH (EC 1.14.13) also referred to as steviol synthases catalyze the conversion of kaurenoic acid into steviol. Examples of enzymes include those of Stevia rebaudiana (accession no. ACD93722), Arabidopsis thaliana (accession no. NP_197872), Vitis vinifera (accession no. XP_002282091), and Medicago trunculata (accession no. ABC59076), and those described in U.S. Patent Application Publication Nos. 2014/0329281, 2014/0357588, 2015/0159188, and WO 2016/038095.

In some embodiments, the host cell includes a heterologous nucleic acid encoding a KAH. In some embodiments, the KAH has an amino acid sequence that is at least 90% (e.g., at least 90%, 91%, 92%, 93%, 94%, 95%, 96%, 97%, 98%, or 99%) identical to the amino acid sequence of SEQ ID NO: 46. In some embodiments, the KAH has an amino acid sequence that is at least 95% (e.g., at least 95%, 96%, 97%, 98%, or 99%) identical to the amino acid sequence of SEQ ID NO: 46. In some embodiments, the KAH has the amino acid sequence of SEQ ID NO: 46.

### Cytochrome P450 Reductase

A CPR (EC 1.6.2.4) is necessary for the activity of KO and/or KAH above. Examples of enzymes include those of Stevia rebaudiana (accession no. ABB88839), Arabidopsis thaliana (accession no. NP_194183), Gibberella fujikuroi (accession no. CAE09055), and Artemisia annua (accession no. ABC47946.1), and those described in U.S. Patent Application Publication Nos. 2014/0329281, 2014/0357588, 2015/0159188, and WO 2016/038095.

In some embodiments, the host cell comprises a heterologous nucleic acid encoding a CPR. In some embodiments, the CPR has an amino acid sequence that is at least 90% (e.g., at least 90%, 91%, 92%, 93%, 94%, 95%, 96%, 97%, 98%, or 99%) identical to the amino acid sequence of SEQ ID NO: 45. In some embodiments, the CPR has an amino acid sequence that is at least 95% (e.g., at least 95%, 96%, 97%, 98%, or 99%) identical to the amino acid sequence of SEQ ID NO: 45. In some embodiments, the CPR has the amino acid sequence of SEQ ID NO: 45.

### Udp Glycosyltransferase

UGT74G1 is capable of functioning as a uridine 5′-diphospho glucosyl: steviol 19-COOH transferase and as a uridine 5′-diphospho glucosyl: steviol-13-O-glucoside 19-COOH transferase. Accordingly, UGT74G1 is capable of converting steviol to 19-glycoside; converting steviol to 19-glycoside, steviolmonoside to rubusoside; and steviolbioside to stevioside. UGT74G1 has been described in Richman et al., 2005, Plant J., vol. 41, pp. 56-67; U.S. Patent Application Publication No. 2014/0329281; WO 2016/038095; and accession no. AAR06920.1.

In some embodiments, the host cell includes a heterologous nucleic acid encoding a UGT74G1. In some embodiments, the UGT74G1 has an amino acid sequence that is at least 90% (e.g., at least 90%, 91%, 92%, 93%, 94%, 95%, 96%, 97%, 98%, or 99%) identical to the amino acid sequence of SEQ ID NO: 37. In some embodiments, the UGT74G1 has an amino acid sequence that is at least 95% (e.g., at least 95%, 96%, 97%, 98%, or 99%) identical to the amino acid sequence of SEQ ID NO: 37. In some embodiments, the UGT74G1 has the amino acid sequence of SEQ ID NO: 37.

UGT76G1 is capable of transferring a glucose moiety to the C-3′ position of an acceptor molecule a steviol glycoside (where glycoside=Glcb(1-2)Glc). This chemistry can occur at either the C-13-O-linked glucose of the acceptor molecule, or the C-19-O-linked glucose acceptor molecule. Accordingly, UGT76G1 is capable of functioning as a uridine 5′-diphospho glucosyltransferase to the: (1)C-3′ position of the 13-O-linked glucose on steviolbioside in a beta linkage forming RebB, (2)C-3′ position of the 19-O-linked glucose on stevioside in a beta linkage forming RebA, and (3)C-3′ position of the 19-O-linked glucose on RebD in a beta linkage forming RebM. UGT76G1 has been described in Richman et al., 2005, Plant J., vol. 41, pp. 56-67; US2014/0329281; WO2016/038095; and accession no. AAR06912.1.

In some embodiments, the UGT76G1 has an amino acid sequence that is at least 90% (e.g., at least 90%, 91%, 92%, 93%, 94%, 95%, 96%, 97%, 98%, or 99%) identical to the amino acid sequence of SEQ ID NO: 39. In some embodiments, the UGT76G1 has an amino acid sequence that is at least 95% (e.g., at least 95%, 96%, 97%, 98%, or 99%) identical to the amino acid sequence of SEQ ID NO: 39. In some embodiments, the UGT76G1 has the amino acid sequence of SEQ ID NO: 39.

UGT85C2 is capable of functioning as a uridine 5′-diphospho glucosyl:steviol 13-OH transferase, and a uridine 5′-diphospho glucosyl:steviol-19-O-glucoside 13-OH transferase. UGT85C2 is capable of converting steviol to steviolmonoside and is also capable of converting 19-glycoside to rubusoside. Examples of UGT85C2 enzymes include those of Stevia rebaudiana: see e.g., Richman et al., (2005), Plant J., vol. 41, pp. 56-67; U.S. Patent Application Publication No. 2014/0329281; WO 2016/038095; and accession no. AAR06916.1.

In some embodiments, the host cell includes a heterologous nucleic acid encoding a UGT85C2. In some embodiments, the UGT85C2 has an amino acid sequence that is at least 90% (e.g., at least 90%, 91%, 92%, 93%, 94%, 95%, 96%, 97%, 98%, or 99%) identical to the amino acid sequence of SEQ ID NO: 36. In some embodiments, the UGT85C2 has an amino acid sequence that is at least 95% (e.g., at least 95%, 96%, 97%, 98%, or 99%) identical to the amino acid sequence of SEQ ID NO: 36. In some embodiments, the UGT85C2 has the amino acid sequence of SEQ ID NO: 36.

UGT40087 is capable of transferring a glucose moiety to the C-2′ position of the 19-O-glucose of RebA to produce RebD. UGT40087 is also capable of transferring a glucose moiety to the C-2′ position of the 19-O-glucose of stevioside to produce RebE. Examples of UGT40087 include those of accession no. XP_004982059.1 and WO 2018/031955.

In some embodiments, the host cell includes a heterologous nucleic acid encoding a UGT40087. In some embodiments, the UGT40087 has an amino acid sequence that is at least 90% (e.g., at least 90%, 91%, 92%, 93%, 94%, 95%, 96%, 97%, 98%, or 99%) identical to the amino acid sequence of SEQ ID NO: 40. In some embodiments, the UGT40087 has an amino acid sequence that is at least 95% (e.g., at least 95%, 96%, 97%, 98%, or 99%) identical to the amino acid sequence of SEQ ID NO: 40. In some embodiments, the UGT40087 has the amino acid sequence of SEQ ID NO: 40.

Mevalonate Pathway Farnesyl Pyrophosphate and/or Geranylgeranyl Pyrophosphate Production

In some embodiments, the host cell provided herein comprises one or more heterologous enzymes of the mevalonate (MEV) pathway, useful for the formation of farnesyl pyrophosphate (FPP) and/or geranylgeranyl pyrophosphate (GGPP). The one or more enzymes of the MEV pathway may include an enzyme that condenses acetyl-CoA with malonyl-CoA to form acetoacetyl-CoA; an enzyme that condenses two molecules of acetyl-CoA to form acetoacetyl-CoA; an enzyme that condenses acetoacetyl-CoA with acetyl-CoA to form HMG-CoA; or an enzyme that converts HMG-CoA to mevalonate. In addition, the genetically modified host cells may include a MEV pathway enzyme that phosphorylates mevalonate to mevalonate 5-phosphate; a MEV pathway enzyme that converts mevalonate 5-phosphate to mevalonate 5-pyrophosphate; a MEV pathway enzyme that converts mevalonate 5-pyrophosphate to isopentenyl pyrophosphate; or a MEV pathway enzyme that converts isopentenyl pyrophosphate to dimethylallyl diphosphate. In particular, the one or more enzymes of the MEV pathway are selected from acetyl-CoA thiolase, acetoacetyl-CoA synthetase, HMG-CoA synthase, HMG-CoA reductase, mevalonate kinase, phosphomevalonate kinase, mevalonate pyrophosphate decarboxylase, and isopentyl diphosphate:dimethylallyl diphosphate isomerase (IDI or IPP isomerase). The genetically modified host cell of the invention may express one or more of the heterologous enzymes of the MEV from one or more heterologous nucleotide sequences comprising the coding sequence of the one or more MEV pathway enzymes.

In some embodiments, the host cell comprises a heterologous nucleic acid encoding an enzyme that can convert isopentenyl pyrophosphate (IPP) into dimethylallyl pyrophosphate (DMAPP). In addition, the host cell may contain a heterologous nucleic acid encoding an enzyme that may condense IPP and/or DMAPP molecules to form a polyprenyl compound. In some embodiments, the genetically modified host cell further contains a heterologous nucleic acid encoding an enzyme that may modify IPP or a polyprenyl to form an isoprenoid compound such as FPP.

The host cell may contain a heterologous nucleic acid that encodes an enzyme that condenses two molecules of acetyl-coenzyme A to form acetoacetyl-CoA (an acetyl-CoA thiolase). Examples of nucleotide sequences encoding acetyl-CoA thiolase include (accession no. NC_000913 REGION: 2324131.2325315 (Escherichia coli)); (D49362 (Paracoccus denitrificans)); and (L20428 (Saccharomyces cerevisiae)).

Acetyl-CoA thiolase catalyzes the reversible condensation of two molecules of acetyl-CoA to yield acetoacetyl-CoA, but this reaction is thermodynamically unfavorable; acetoacetyl-CoA thiolysis is favored over acetoacetyl-CoA synthesis. Acetoacetyl-CoA synthase (AACS) (also referred to as acetyl-CoA:malonyl-CoA acyltransferase; EC 2.3.1.194) condenses acetyl-CoA with malonyl-CoA to form acetoacetyl-CoA. In contrast to acetyl-CoA thiolase, AACS-catalyzed acetoacetyl-CoA synthesis is essentially an energy-favored reaction, due to the associated decarboxylation of malonyl-CoA. In addition, AACS exhibits no thiolysis activity against acetoacetyl-CoA, and thus the reaction is irreversible.

In cells expressing acetyl-CoA thiolase and a heterologous ADA and/or phosphotransacetylase (PTA), the reversible reaction catalyzed by acetyl-CoA thiolase, which favors acetoacetyl-CoA thiolysis, may result in a large acetyl-CoA pool. In view of the reversible activity of ADA, this acetyl-CoA pool may in turn drive ADA towards the reverse reaction of converting acetyl-CoA to acetaldehyde, thereby diminishing the benefits provided by ADA towards acetyl-CoA production. Similarly, the activity of PTA is reversible, and thus, a large acetyl-CoA pool may drive PTA towards the reverse reaction of converting acetyl-CoA to acetyl phosphate. Therefore, in some embodiments, in order to provide a strong pull on acetyl-CoA to drive the forward reaction of ADA and PTA, the MEV pathway of the genetically modified host cell provided herein utilizes an acetoacetyl-CoA synthase to form acetoacetyl-CoA from acetyl-CoA and malonyl-CoA.

The AACS obtained from Streptomyces sp. Strain CL190 may be used (see Okamura et al., (2010), PNAS, vol. 107, pp. 11265-11270). Representative AACS encoding nucleic acids sequences from Streptomyces sp. Strain CL190 include the sequence of Accession No. AB540131.1, and the corresponding AACS protein sequences include the sequence of Accession Nos. D7URV0 and BAJ10048. Other acetoacetyl-CoA synthases useful for the invention include those of Streptomyces sp. (see Accession Nos. AB183750; KO-3988 BAD86806; KO-3988 AB212624; and KO-2988 BAE78983); S. anulatus strain 9663 (see Accession Nos. FN178498 and CAX48662); Actinoplanes sp. A40644 (see Accession Nos. AB113568 and BAD07381); Streptomyces sp. C (see accession nos. NZ_ACEW010000640 and ZP_05511702); Nocardiopsis dassonvillei DSM 43111 (see Accession Nos. NZ_ABUIl01000023 and ZP_04335288); Mycobacterium ulcerans Agy99 (see Accession Nos. NC_008611 and YP_907152); Mycobacterium marinum M (see Accession Nos. NC_010612 and YP_001851502); Streptomyces sp. Mg1 (see Accession Nos. NZ_DS570501 and ZP_05002626); Streptomyces sp. AA4 (see Accession Nos. NZ_ACEV01000037 and ZP_05478992); S. roseosporus NRRL 15998 (see Accession Nos. NZ_ABYB01000295 and ZP_04696763); Streptomyces sp. ACTE (see Accession Nos. NZ_ADFD01000030 and ZP_06275834); S. viridochromogenes DSM 40736 (see Accession Nos. NZ_ACEZ01000031 and ZP_05529691); Frankia sp. Cc13 (see Accession Nos. NC_007777 and YP_480101); Nocardia brasiliensis (see Accession Nos. NC_018681 and YP_006812440.1); and Austwickia chelonae (see Accession Nos. NZ_BAGZ01000005 and ZP_10950493.1). Additional suitable acetoacetyl-CoA synthases include those described in U.S. Patent Application Publication Nos. 2010/0285549 and 2011/0281315.

Acetoacetyl-CoA synthases also useful in the compositions and methods provided herein include those molecules which are said to be “derivatives” of any of the acetoacetyl-CoA synthases described herein. Such a “derivative” has the following characteristics: (1) it shares substantial homology with any of the acetoacetyl-CoA synthases described herein; and (2) is capable of catalyzing the irreversible condensation of acetyl-CoA with malonyl-CoA to form acetoacetyl-CoA. A derivative of an acetoacetyl-CoA synthase is said to share “substantial homology” with acetoacetyl-CoA synthase if the amino acid sequences of the derivative is at least 80%, and more preferably at least 90%, and most preferably at least 95%, the same as that of acetoacetyl-CoA synthase.

In some embodiments, the host cell comprises a heterologous nucleotide sequence encoding an enzyme that can condense acetoacetyl-CoA with another molecule of acetyl-CoA to form 3-hydroxy-3-methylglutaryl-CoA (HMG-CoA), e.g., an HMG-CoA synthase. Examples of nucleotide sequences encoding such an enzyme include: (NC_001145. complement 19061.20536; Saccharomyces cerevisiae), (X96617; Saccharomyces cerevisiae), (X83882; Arabidopsis thaliana), (AB037907; Kitasatospora griseola), (BT007302; Homo sapiens), and (NC_002758, Locus tag SAV2546, GenelD 1122571; Staphylococcus aureus).

In some embodiments, the host cell comprises a heterologous nucleotide sequence encoding an enzyme that can convert HMG-CoA into mevalonate, e.g., an HMG-CoA reductase. The HMG-CoA reductase may be an NADH-using hydroxymethylglutaryl-CoA reductase-CoA reductase. HMG-CoA reductases (EC 1.1.1.34; EC 1.1.1.88) catalyze the reductive deacylation of (S)-HMG-CoA to (R)-mevalonate, and can be categorized into two classes, class I and class II HMGrs. Class I includes the enzymes from eukaryotes and most archaea, and class 11 includes the HMG-CoA reductases of certain prokaryotes and archaea. In addition to the divergence in the sequences, the enzymes of the two classes also differ with regard to their cofactor specificity. Unlike the class I enzymes, which utilize NADPH exclusively, the class II HMG-CoA reductases vary in the ability to discriminate between NADPH and NADH (See, e.g., Hedl et al., (2004) Journal of Bacteriology, vol. 186, pp. 1927-1932). Co-factor specificities for select class II HMG-CoA reductases are provided in Table 2.

TABLE 2

| | Coenzyme | | |
|---|---|---|---|
| Source | specificity | Kₘ^{NADPH} (μM) | Kₘ^{NADH} (μM) |
| P. mevalonii | NADH | | 80 |
| A. fulgidus | NAD(P)H | 500 | 160 |
| S. aureus | NAD(P)H | 70 | 100 |
| E. faecalis | NADPH | 30 | |

HMG-CoA reductases useful for the invention include HMG-CoA reductases that are capable of utilizing NADH as a cofactor, e.g., HMG-CoA reductase from P. mevalonii, A. fulgidus, or S. aureus. In particular embodiments, the HMG-CoA reductase is capable of only utilizing NADH as a cofactor, e.g., HMG-CoA reductase from P. mevalonii, S. pomeroyi, or D. acidovorans.

In some embodiments, the NADH-using HMG-CoA reductase is from Pseudomonas mevalonii. The sequence of the wild-type mvaA gene of Pseudomonas mevalonii, which encodes HMG-CoA reductase (EC 1.1.1.88), has been previously described (see Beach and Rodwell, (1989), J. Bacteriol., vol. 171, pp. 2994-3001). Representative mvaA nucleotide sequences of Pseudomonas mevalonii include accession number M24015. Representative HMG-CoA reductase protein sequences of Pseudomonas mevalonii include accession numbers AAA25837, P13702, and MVAA_PSEMV.

In some embodiments, the NADH-using HMG-CoA reductase is from Silicibacter pomeroyi. Representative HMG-CoA reductase nucleotide sequences of Silicibacter pomeroyi include accession number NC_006569.1. Representative HMG-CoA reductase protein sequences of Silicibacter pomeroyi include accession number YP_164994.

In some embodiments, the NADH-using HMG-CoA reductase is from Delftia acidovorans. A representative HMG-CoA reductase nucleotide sequences of Delftia acidovorans includes NC_010002 REGION: complement (319980 . . . 321269). Representative HMG-CoA reductase protein sequences of Delftia acidovorans include accession number YP_001561318.

In some embodiments, the NADH-using HMG-CoA reductase is from Solanum tuberosum (see Crane et al., (2002), J. Plant Physiol., vol. 159, pp. 1301-1307).

NADH-using HMG-CoA reductases useful in the practice of the invention also include those molecules which are said to be “derivatives” of any of the NADH-using HMG-CoA reductases described herein, e.g., from P. mevalonii, S. pomeroyi and D. acidovorans. Such a “derivative” has the following characteristics: (1) it shares substantial homology with any of the NADH-using HMG-CoA reductases described herein; and (2) is capable of catalyzing the reductive deacylation of (S)-HMG-CoA to (R)-mevalonate while preferentially using NADH as a cofactor. A derivative of an NADH-using HMG-CoA reductase is said to share “substantial homology” with NADH-using HMG-CoA reductase if the amino acid sequences of the derivative is at least 80%, and more preferably at least 90%, and most preferably at least 95%, the same as that of NADH-using HMG-CoA reductase.

As used herein, the phrase “NADH-using” means that the NADH-using HMG-CoA reductase is selective for NADH over NADPH as a cofactor, for example, by demonstrating a higher specific activity for NADH than for NADPH. The selectivity for NADH as a cofactor is expressed as a k_{cat}(^{NADH})/k_{cat}^{(NADPH)} ratio. The NADH-using HMG-CoA reductase of the invention may have a k_{cat}(^{NADH})/k_{cat}^{(NADPH)} ratio of at least 5, 10, 15, 20, 25 or greater than 25. The NADH-using HMG-CoA reductase may use NADH exclusively. For example, an NADH-using HMG-CoA reductase that uses NADH exclusively displays some activity with NADH supplied as the sole cofactor in vitro, and displays no detectable activity when NADPH is supplied as the sole cofactor. Any method for determining cofactor specificity known in the art can be utilized to identify HMG-CoA reductases having a preference for NADH as cofactor (see e.g., (Kim et al., (2000), Protein Science, vol. 9, pp. 1226-1234) and (Wilding et al., (2000), J. Bacteriol., vol. 182, pp. 5147-5152).

In some cases, the NADH-using HMG-CoA reductase is engineered to be selective for NADH over NAPDH, for example, through site-directed mutagenesis of the cofactor-binding pocket. Methods for engineering NADH-selectivity are described in Watanabe et al., (2007), Microbiology, vol. 153, pp. 3044-3054), and methods for determining the cofactor specificity of HMG-CoA

reductases are described in Kim et al., (2000), Protein Sci., vol. 9, pp. 1226-1234). The NADH-using HMG-CoA reductase may be derived from a host species that natively comprises a mevalonate degradative pathway, for example, a host species that catabolizes mevalonate as its sole carbon source. In these cases, the NADH-using HMG-CoA reductase, which normally catalyzes the oxidative acylation of internalized (R)-mevalonate to (S)-HMG-CoA within its native host cell, is utilized to catalyze the reverse reaction, that is, the reductive deacylation of (S)-HMG-CoA to (R)-mevalonate, in a genetically modified host cell comprising a mevalonate biosynthetic pathway. Prokaryotes capable of growth on mevalonate as their sole carbon source have been described by: (Anderson et al., (1989), J. Bacteriol, vol. 171, pp. 6468-6472); (Beach et al., (1989), J. Bacteriol., vol. 171, pp. 2994-3001); Bensch et al., J. Biol. Chem., vol. 245, pp. 3755-3762); (Fimongnari et al., (1965), Biochemistry, vol. 4, pp. 2086-2090); Siddiqi et al., (1962), Biochem. Biophys. Res. Commun., vol. 8, pp. 110-113); (Siddiqi et al., (1967), J. Bacteriol., vol. 93, pp. 207-214); and (Takatsuji et al., (1983), Biochem. Biophys. Res. Commun., vol. 110, pp. 187-193).

The host cell may contain both a NADH-using HMGr and an NADPH-using HMG-CoA reductase. Examples of nucleotide sequences encoding an NADPH-using HMG-CoA reductase include: (NM_206548; Drosophila melanogaster), (NC_002758, Locus tag SAV2545, GenelD 1122570; Staphylococcus aureus), (AB015627; Streptomyces sp. KO 3988), (AX128213, providing the sequence encoding a truncated HMG-CoA reductase; Saccharomyces cerevisiae), and (NC_001145: complement (115734.118898; Saccharomyces cerevisiae).

The host cell may contain a heterologous nucleotide sequence encoding an enzyme that can convert mevalonate into mevalonate 5-phosphate, e.g., a mevalonate kinase. Illustrative examples of nucleotide sequences encoding such an enzyme include: (L77688; Arabidopsis thaliana) and (X55875; Saccharomyces cerevisiae).

The host cell may contain a heterologous nucleotide sequence encoding an enzyme that can convert mevalonate 5-phosphate into mevalonate 5-pyrophosphate, e.g., a phosphomevalonate kinase. Illustrative examples of nucleotide sequences encoding such an enzyme include: (AF429385; Hevea brasiliensis), (NM_006556; Homo sapiens), and (NC_001145. complement 712315.713670; Saccharomyces cerevisiae).

The host cell may contain a heterologous nucleotide sequence encoding an enzyme that can convert mevalonate 5-pyrophosphate into isopentenyl diphosphate (IPP), e.g., a mevalonate pyrophosphate decarboxylase. Illustrative examples of nucleotide sequences encoding such an enzyme include: (X97557; Saccharomyces cerevisiae), (AF290095; Enterococcus faecium), and (U49260; Homo sapiens).

The host cell may contain a heterologous nucleotide sequence encoding an enzyme that can convert IPP generated via the MEV pathway into dimethylallyl pyrophosphate (DMAPP), e.g., an IPP isomerase. Illustrative examples of nucleotide sequences encoding such an enzyme include: (NC_000913, 3031087.3031635; Escherichia coli), and (AF082326; Haematococcus pluvialis).

In some embodiments, the host cell further comprises a heterologous nucleotide sequence encoding a polyprenyl synthase that can condense IPP and/or DMAPP molecules to form polyprenyl compounds containing more than five carbons.

The host cell may contain a heterologous nucleotide sequence encoding an enzyme that can condense one molecule of IPP with one molecule of DMAPP to form one molecule of geranyl pyrophosphate (GPP), e.g., a GPP synthase. Non-limiting examples of nucleotide sequences encoding such an enzyme include: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia brewen), (AY953508; lps pini), (DQ286930; Lycopersicon esculentum), (AF182828; Mentha×piperita), (AF182827; Mentha×piperita), (MP1249453; Mentha×piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

The host cell may contain a heterologous nucleotide sequence encoding an enzyme that can condense two molecules of IPP with one molecule of DMAPP, or add a molecule of IPP to a molecule of GPP, to form a molecule of farnesyl pyrophosphate (“FPP”), e.g., an FPP synthase. Non-limiting examples of nucleotide sequences that encode an FPP synthase include: (ATU80605; Arabidopsis thaliana), (ATHFPS2R; Arabidopsis thaliana), (AAU36376; Artemisia annua), (AF461050; Bos taurus), (D00694; Escherichia coli K-12), (AE009951, Locus AAL95523; Fusobacterium nucleatum subsp. nucleatum ATCC 25586), (GFFPPSGEN; Gibberella fujikuroi), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_008022, Locus YP_598856; Streptococcus pyogenes MGAS10270), (NC_008023, Locus YP_600845; Streptococcus pyogenes MGAS2096), (NC_008024, Locus YP_602832; Streptococcus pyogenes MGAS10750), (MZEFPS; Zea mays), (AE000657, Locus AAC06913; Aquifex aeolicus VF5), (NM_202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_002940, Locus NP_873754; Haemophilus ducreyi35000HP), (L42023, Locus AAC23087; Haemophilus influenzae Rd KW20), (J05262; Homo sapiens), (YP_395294; Lactobacillus sakei subsp. sakei23K), (NC_005823, Locus YP_000273; Leptospira interrogans serovar Copenhageni str. Fiocruz L1-130), (AB003187; Micrococcus luteus), (NC_002946, Locus YP_208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_004556, Locus NP 779706; Xylella fastidiosa Temeculal).

In addition, the host cell may contain a heterologous nucleotide sequence encoding an enzyme that can combine IPP and DMAPP or IPP and FPP to form GGPP. Non-limiting examples of nucleotide sequences that encode such an enzyme include: (ATHGERPYRS; Arabidopsis thaliana), (BT005328; Arabidopsis thaliana), (NM_119845; Arabidopsis thaliana), (NZ_AAJM01000380, Locus ZP_00743052; Bacillus thuringiensis serovar israelensis, ATCC 35646 sq1563), (CRGGPPS; Catharanthus roseus), (NZ_AABF02000074, Locus ZP_00144509; Fusobacterium nucleatum subsp. vincentii, ATCC 49256), (GFGGPPSGN; Gibberella fujikurol), (AY371321; Ginkgo biloba), (AB055496; Hevea brasiliensis), (AB017971; Homo sapiens), (MCl276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_007759, Locus YP_461832; Syntrophus aciditrophicus SB), (NC_006840, Locus YP_204095; Vibrio fischeri ES114), (NM_112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_004350, Locus NP_721015; Streptococcus mutans UA159).

While examples of the enzymes of the mevalonate pathway are described above, in certain embodiments, enzymes of the 1-deoxy-D-xylulose 5-phosphate (DXP) pathway can be used as an alternative or additional pathway to produce DMAPP and IPP in the host cells, compositions and methods described herein. Enzymes and nucleic acids encoding the enzymes of the DXP pathway are well-known and characterized in the art, e.g., WO 2012/135591.

### Exemplary Cell Strains

Host cells of the invention provided herein include archae, prokaryotic, and eukaryotic cells.

Suitable prokaryotic host cells include, but are not limited to, any of a gram-positive, gram-negative, and gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphylococcus, Streptomyces, Synechococcus, and Zymomonas. Examples of prokaryotic strains include, but are not limited to: Bacillus subtilis, Bacillus amyloliquefacines, Brevibacterium ammoniagenes, Brevibacterium immariophilum, Clostridium beijerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the host cell is an Escherichia colicell.

Suitable archae hosts include, but are not limited to, cells belonging to the genera: Aeropyrum, Archaeoglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic hosts include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malasserzia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastoporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma.

In some embodiments, the host cell is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the host cell is a strain of the genus Candida, such as Candida lipolytica, Candida guilliermondii, Candida krusei, Candida pseudotropicalis, or Candida utils.

In preferred embodiments, the host cell is Saccharomyces cerevisiae. In some embodiments, the host is a strain of Saccharomyces cerevisiae selected from Baker's yeast, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1 BR-1, BR-2, ME-2, VR-2, MA-3, MA-4, CAT-1, CB-1, NR-1, BT-1, and AL-1. In some embodiments, the host cell is a strain of Saccharomyces cerevisiae selected from PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

### Gene Expression Regulatory Elements

In some embodiments, the genetically modified host cell includes a promoter that regulates the expression and/or stability of at least one of the one or more heterologous nucleic acids. In certain aspects, the promoter negatively regulates the expression and/or stability of the at least one heterologous nucleic acid. In some embodiments, the host cell is a yeast cell. The promoter can be responsive to a small molecule that can be present in the culture medium of a fermentation of the modified yeast. In some embodiments, the small molecule is maltose or an analog or derivative thereof. In some embodiments, the small molecule is lysine or an analog or derivative thereof. Maltose and lysine can be attractive selections for the small molecule as they are relatively inexpensive, non-toxic, and stable.

In some embodiments, the promoter that regulates expression of the variant UDP glycosyltransferase polypeptide, e.g., the polypeptide of any one of SEQ ID NO: 2-30, is a relatively weak promoter, or an inducible promoter. Illustrative promoters include, for example, lower-strength GAL pathway promoters, such as GAL10, GAL2, and GAL3 promoters. Additional illustrative promoters for expressing a UDP glycosyltransferase polypeptide include constitutive promoters from S. cerevisiae native promoters, such as the promoter from the native TDH3 gene. In some embodiments, a lower strength promoter provides a decrease in expression of at least 25%, or at least 30%, 40%, or 50%, or greater, when compared to a GAL1 promoter.

Expression of a variant UDP glycosyltransferase polypeptide, e.g., the polypeptide of any one of SEQ ID NO: 2-30 can be accomplished by introducing into the host cells a nucleic acid including a nucleotide sequence encoding the variant UDP glycosyltransferase polypeptide under the control of regulatory elements that permit expression in the host cell. In some embodiments, the nucleic acid is included in an extrachromosomal plasmid. In other embodiments, the nucleic acid is included in a chromosomal integration vector that can integrate the nucleotide sequence into the chromosome of the host cell. Expression of a polypeptide of any one of SEQ ID NO: 2-30, or a variant thereof as described herein can be achieved by using parallel methodology.

### Heterologous Nucleic Acids

In some embodiments, the one or more heterologous nucleic acids are introduced into the genetically modified host cells by using a gap repair molecular biology technique. In some embodiments, the host cell is a yeast cell. In these methods, if the yeast has non-homologous end joining (NHEJ) activity, as is the case for Kluyveromyces marxianus, then the NHEJ activity in the yeast can be first disrupted in any of a number of ways. Further details related to genetic modification of yeast cells through gap repair can be found in U.S. Pat. No. 9,476,065, the full disclosure of which is incorporated by reference herein in its entirety for all purposes.

In some embodiments, the one or more heterologous nucleic acids are introduced into the genetically modified host cells by using one or more site-specific nucleases, which are capable of causing breaks at designated regions within selected nucleic acid target sites. Examples of such nucleases include, but are not limited to, endonucleases, site-specific recombinases, transposases, topoisomerases, zinc finger nucleases, TAL-effector DNA binding domain-nuclease fusion proteins (TALENs), CRISPR/Cas-associated RNA-guided endonucleases, and meganucleases. Further details related to genetic modification of yeast cells through site specific nuclease activity can be found in U.S. Pat. No. 9,476,065, the full disclosure of which is incorporated by reference herein in its entirety for all purposes.

### Nucleic Acid and Amino Acid Sequence Optimization

Described herein are specific genes and proteins useful in the methods, compositions, and organisms of the disclosure; however, it will be recognized that absolute identity to such genes is not necessary. For example, changes in a particular gene or polynucleotide including a sequence encoding a polypeptide or enzyme can be performed and screened for activity. Typically, such changes include conservative mutations and silent mutations. Such modified or mutated polynucleotides and polypeptides can be screened for expression of a functional enzyme using methods known in the art. Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17: 477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24: 216-8).

Those of skill in the art will recognize that, due to the degenerate nature of the genetic code, a variety of DNA molecules differing in their nucleotide sequences can be used to encode a given heterologous polypeptide of the disclosure. A native DNA sequence encoding the biosynthetic enzymes described above is referenced herein merely to illustrate an embodiment of the disclosure, and the disclosure includes DNA molecules of any sequence that encodes the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or without significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties, e.g., charge or hydrophobicity. In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol. Biol. 25: 365-89).

Furthermore, any of the genes encoding the foregoing enzymes (or any others mentioned herein (or any of the regulatory elements that control or modulate expression thereof) can be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., Salmonella spp., or X. dendrorhous.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art can be suitable to identify analogous genes and analogous enzymes. Techniques include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, e.g., as described herein or in Kiritani, K., Branched-Chain Amino Acids Methods Enzymology, 1970; then isolating the enzyme with said activity through purification; determining the protein sequence of the enzyme through techniques such as Edman degradation; design of PCR primers to the likely nucleic acid sequence; amplification of said DNA sequence through PCR; and cloning of said nucleic acid sequence. To identify homologous or similar genes and/or homologous or similar enzymes, suitable techniques also include comparison of data concerning a candidate gene or enzyme with databases such as BRENDA, KEGG, or MetaCYC. The candidate gene or enzyme can be identified within the above-mentioned databases in accordance with the teachings herein.

### Methods of Producing Steviol Glycosides

Also provided herein are methods of producing one or more steviol glycosides (e.g., RebA, RebB, RebD, RebE, or RebM). For example, provided herein are methods for the production RebM. The methods may include, for example, providing a population of host cells (e.g., yeast cell) capable of producing one or more steviol glycosides (e.g., RebA, RebB, RebD, RebE, or RebM), wherein the host cells are genetically modified to express a variant UDP glycosyltransferase polypeptide, e.g., a polypeptide having the amino acid sequence of any one of SEQ ID NOs: 2-30 herein. Each host cell (e.g., yeast cell) of the population may include a heterologous nucleic acid that encodes a variant UDP glycosyltransferase polypeptide. In some embodiments, the population includes any of the host cells (e.g., yeast cells) as disclosed herein and discussed above. Further, the methods described herein include providing a culture medium and culturing the host cells in the culture medium under conditions suitable for the host cells to produce one or more steviol glycosides.

The culturing can be performed in a suitable culture medium in a suitable container, including but not limited to a cell culture plate, a flask, or a fermentor. Any suitable fermentor may be used, including, but not limited to, a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric et al., in Ullmann's Encyclopedia of Industrial Chemistry, Sixth Edition, Volume 12, pages 398-473, Wiley-VCH Verlag GmbH & Co.

KDaA, Weinheim, Germany. Further, the methods can be performed at any scale of fermentation known in the art to support industrial production of microbial products. Materials and methods for the maintenance and growth of cell cultures are well known to those skilled in the art of microbiology or fermentation science (see, for example, Bailey et al., Biochemical Engineering Fundamentals, second edition, McGraw Hill, New York, 1986). Consideration should be given to appropriate culture medium, pH, temperature, and requirements for aerobic, microaerobic, or anaerobic conditions, depending on the specific requirements of the host cell, the fermentation, and the process.

In some embodiments, the culturing is carried out for a period of time sufficient for the transformed population to undergo a plurality of doublings until a desired cell density is reached. In some embodiments, the culturing is carried out for a period of time sufficient for the host cell population to reach a cell density (OD600) of between 0.01 and 400 in the fermentation vessel or container in which the culturing is being carried out. The culturing can be carried out until the cell density is, for example, between 0.1 and 14, between 0.22 and 33, between 0.53 and 76, between 1.2 and 170, or between 2.8 and 400. In terms of upper limits, the culturing can be carried until the cell density is no more than 400, e.g., no more than 170, no more than 76, no more than 33, no more than 14, no more than 6.3, no more than 2.8, no more than 1.2, no more than 0.53, or no more than 0.23. In terms of lower limits, the culturing can be carried out until the cell density is greater than 0.1, e.g., greater than 0.23, greater than 0.53, greater than 1.2, greater than 2.8, greater than 6.3, greater than 14, greater than 33, greater than 76, or greater than 170. Higher cell densities, e.g., greater than 400, and lower cell densities, e.g., less than 0.1, are also contemplated.

In other embodiments, the culturing is carried for a period of time, for example, between 12 hours and 92 hours, e.g., between 12 hours and 60 hours, between 20 hours and 68 hours, between 28 hours and 76 hours, between 36 hours and 84 hours, or between 44 hours and 92 hours. In some embodiments, the culturing is carried out for a period of time, for example, between 5 days and 20 days, e.g., between 5 days and 14 days, between 6.5 days and 15.5 days, between 8 days and 17 days, between 9.5 days and 18.5 days, or between 11 days and 20 days. In terms of upper limits, the culturing can be carried out for less than 20 days, e.g., less than 18.5 days, less than 17 days, less than 15.5 days, less than 14 days, less than 12.5 day, less than 11 days, less than 9.5 days, less than 8 days, less than 6.5 days, less than 5 day, less than 92 hours, less than 84 hours, less than 76 hours, less than 68 hours, less than 60 hours, less than 52 hours, less than 44 hours, less than 36 hours, less than 28 hours, or less than 20 hours. In terms of lower limits, the culturing can be carries out for greater than 12 hours, e.g., greater than 20 hours, greater than 28 hours, greater than 36 hours, greater than 44 hours, greater than 52 hours, greater than 60 hours, greater than 68 hours, greater than 76 hours, greater than 84 hours, greater than 92 hours, greater than 5 days, greater than 6.5 days, greater than 8 days, greater than 9.5 days, greater than 11 days, greater than 12.5 days, greater than 14 days, greater than 15.5 days, greater than 17 days, or greater than 18.5 days. Longer culturing times, e.g., greater than 20 days, and shorter culturing times, e.g., less than 5 hours, are also contemplated.

In certain embodiments, the production of the one or more steviol glycosides by the population of host cells (e.g., yeast cells) is inducible by an inducing compound. Such yeast can be manipulated with ease in the absence of the inducing compound. The inducing compound is then added to induce the production of one or more steviol glycosides by the yeast. In other embodiments, production of the one or more steviol glycosides by the yeast is inducible by changing culture conditions, such as, for example, the growth temperature, media constituents, and the like.

In certain embodiments, an inducing agent is added during a production stage to activate a promoter or to relieve repression of a transcriptional regulator associated with a biosynthetic pathway to promote production of one or more steviol glycosides. In certain embodiments, an inducing agent is added during a build stage to repress a promoter or to activate a transcriptional regulator associated with a biosynthetic pathway to repress the production of one or more steviol glycosides, and an inducing agent is removed during the production stage to activate a promoter to relieve repression of a transcriptional regulator to promote the production of one or more steviol glycosides.

As discussed above, in some embodiments, the provided host cell includes a promoter that regulates the expression and/or stability of the heterologous nucleic acid. Thus, in certain embodiments, the promoter can be used to control the timing of gene expression and/or stability of proteins, for example, a UDP glycosyltransferase polypeptide, e.g., the polypeptide of any one of SEQ ID NO: 2-30 described herein.

In some embodiments, when fermentation of a host cell (e.g., yeast cell) is carried out in the presence of a small molecule, e.g., at least about 0.1% maltose or lysine, steviol glycoside production is substantially reduced or turned off. When the amount of the small molecule in the fermentation culture medium is reduced or eliminated, steviol glycoside production is turned on or increased. Such a system enables the use of the presence or concentration of a selected small molecule in a fermentation medium as a switch for the production of non-catabolic, e.g., RebA, RebB, RebD, RebE, or RebM, compounds. Controlling the timing of non-catabolic compound production to occur only when production is desired redirects the carbon flux during the non-production phase into cell maintenance and biomass. This more efficient use of carbon can greatly reduce the metabolic burden on the host cells, improve cell growth, increase the stability of the heterologous genes, reduce strain degeneration, and/or contribute to better overall health and viability of the cells.

In some embodiments, the fermentation method includes a two-step process that utilizes a small molecule as a switch to affect the “off” and “on” stages. In the first step, i.e., the “build” stage, step (a) wherein production of the compound is not desired, the genetically modified yeast is grown in a growth or “build” medium including the small molecule in an amount sufficient to induce the expression of genes under the control of a responsive promoter, and the induced gene products act to negatively regulate production of the non-catabolic compound. After transcription of the fusion DNA construct under the control of a maltose-responsive or lysine-responsive promoter, the stability of the fusion proteins is post-translationally controlled. In the second step, i.e., the “production” stage, step (b), the fermentation is carried out in a culture medium including a carbon source wherein the small molecule is absent or in sufficiently low amounts such that the activity of a responsive promoter is reduced or inactive and the fusion proteins are destabilized. As a result, the production of the heterologous non-catabolic compound by the host cells is turned on or increased.

In some embodiments, the culture medium is any culture medium in which a host cell (e.g., yeast cell) capable of producing a steviol glycoside (e.g., RebA, RebB, RebD, RebE, or RebM) can subsist, i.e., maintain growth and viability. In some embodiments, the culture medium is an aqueous medium including assimilable carbon, nitrogen, and phosphate sources. Such a medium can also include appropriate salts, minerals, metals, and other nutrients. In some embodiments, the carbon source and each of the essential cell nutrients, are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

In another embodiment, the method of producing one or more steviol glycosides includes culturing host cells in separate build and production culture media. For example, the method can include culturing the genetically modified host cell in a build stage wherein the cell is cultured under non-producing conditions, e.g., non-inducing conditions, to produce an inoculum, then transferring the inoculum into a second fermentation medium under conditions suitable to induce production of one or more steviol glycosides, e.g., inducing conditions, and maintaining steady state conditions in the second fermentation stage to produce a cell culture containing steviol glycosides (e.g., RebA, RebB, RebD, RebE, or RebM).

Suitable conditions and suitable media for culturing microorganisms are well known in the art. For example, the suitable medium may be supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

The carbon source may be a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose, in the culture medium may be sufficient to promote cell growth but is not so high as to repress growth of the microorganism used. Typically, cultures are run with a carbon source, such as glucose, being added at levels to achieve the desired level of growth and biomass. The concentration of a carbon source, such as glucose, in the culture medium may be greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

The concentration of a carbon source, such as glucose, in the culture medium may be sufficient to promote cell growth but is not so high as to repress growth of the microorganism used. Typically, cultures are run with a carbon source, such as glucose, being added at levels to achieve the desired level of growth and biomass. The concentration of a carbon source, such as glucose, in the culture medium may be greater than about 1 g/L, preferably greater than about 2 g/L, and more preferably greater than about 5 g/L. In addition, the concentration of a carbon source, such as glucose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. In some embodiments, the addition of a nitrogen source to the culture medium beyond a certain concentration is not advantageous for the growth of the yeast. As a result, the concentration of the nitrogen sources, in the culture medium can be less than about 20 g/L, e.g., less than about 10 g/L or less than about 5 g/L. Further, in some instances it may be desirable to allow the culture medium to become depleted of the nitrogen sources during culturing.

The effective culture medium can contain other compounds such as inorganic salts, vitamins, trace metals or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, e.g., greater than about 2.0 g/L or greater than about 5.0 g/L. In some embodiments, the addition of phosphate to the culture medium beyond certain concentrations is not advantageous for the growth of the yeast. Accordingly, the concentration of phosphate in the culture medium can be less than about 20 g/L, e.g., less than about 15 g/L or less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, e.g., greater than about 1.0 g/L or greater than about 2.0 g/L. In some embodiments, the addition of magnesium to the culture medium beyond certain concentrations is not advantageous for the growth of the yeast. Accordingly, the concentration of magnesium in the culture medium can be less than about 10 g/L, e.g, less than about 5 g/L or less than about 3 g/L. Further, in some instances it may be desirable to allow the culture medium to become depleted of a magnesium source during culturing.

In some embodiments, the culture medium can also include a biologically acceptable chelating agent, such as the dihydrate of trisodium citrate. In such instance, the concentration of a chelating agent in the culture medium can be greater than about 0.2 g/L, e.g., greater than about 0.5 g/L or greater than about 1 g/L. In some embodiments, the addition of a chelating agent to the culture medium beyond certain concentrations is not advantageous for the growth of the yeast. Accordingly, the concentration of a chelating agent in the culture medium can be less than about 10 g/L, e.g., less than about 5 g/L or less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, e.g., within the range of from about 20 mg/L to about 1000 mg/L or in the range of from about 50 mg/L to about 500 mg/L.

The culture medium can also include sodium chloride. Typically, the concentration of sodium chloride in the culture medium is within the range of from about 0.1 g/L to about 5 g/L, e.g., within the range of from about 1 g/L to about 4 g/L or in the range of from about 2 g/L to about 4 g/L.

In some embodiments, the culture medium can also include trace metals. Such trace metals can be added to the culture medium as a stock solution that, for convenience, can be prepared separately from the rest of the culture medium Typically, the amount of such a trace metals solution added to the culture medium is greater than about 1 ml/L, e.g., greater than about 5 mL/L, and more preferably greater than about 10 mL/L. In some embodiments, the addition of a trace metals to the culture medium beyond certain concentrations is not advantageous for the growth of the yeast. Accordingly, the amount of such a trace metals solution added to the culture medium can be less than about 100 mL/L, e.g., less than about 50 mL/L or less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, inositol, pyridoxine-HCl, thiamine-HCl, and combinations thereof. Such vitamins can be added to the culture medium as a stock solution that, for convenience, can be prepared separately from the rest of the culture medium In some embodiments, the addition of vitamins to the culture medium beyond certain concentrations is not advantageous for the growth of the yeast.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, e.g., during the production stage of the fermentation. In some embodiments, the culture may be supplemented with relatively high concentrations of such components at the outset, for example, of the production stage, so that growth and/or steviol glycoside production (e.g., steviol glycoside production) is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those of ordinary skill in the art, the rate of consumption of nutrient increases during culture as the cell density of the medium increases. Moreover, to avoid introduction of foreign microorganisms into the culture medium, addition can be performed using aseptic addition methods, as are known in the art. In addition, an anti-foaming agent may be added during the culture.

The temperature of the culture medium can be any temperature suitable for growth of the genetically modified yeast population and/or production of the one or more steviol glycosides (e.g., RebA, RebB, RebD, RebE, or RebM). For example, prior to inoculation of the culture medium with an inoculum, the culture medium can be brought to and maintained at a temperature in the range of from about 20° C. to about 45° C., e.g., to a temperature in the range of from about 25° C. to about 40° C. or of from about 28° C. to about 32° C. For example, the culture medium can be brought to and maintained at a temperature of 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C.

The pH of the culture medium can be controlled by the addition of acid or base to the culture medium In such cases when ammonia is used to control pH, it also conveniently serves as a nitrogen source in the culture medium. In some embodiments, the pH is maintained from about 3.0 to about 8.0, e.g., from about 3.5 to about 7.0 or from about 4.0 to about 6.5.

The carbon source concentration, such as the glucose concentration, of the culture medium is monitored during culture. Glucose concentration of the culture medium can be monitored using known techniques, such as, for example, use of the glucose oxidase enzyme test or high-pressure liquid chromatography, which can be used to monitor glucose concentration in the supernatant, e.g., a cell-free component of the culture medium. The carbon source concentration is typically maintained below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g., the nitrogen and phosphate sources) can be maintained simultaneously. Likewise, the trace metals concentrations can be maintained in the culture medium by addition of aliquots of the trace metals solution.

Other suitable fermentation medium and methods are described in, e.g., WO 2016/196321.

In some embodiments, the host cells (e.g., yeast cells) produce RebM. The concentration of produced RebM in the culture medium can be, for example, between 1 g/I and 125 g/I, e.g., between 5 g/I and 115 g/I, between 10 g/I and 110 g/I, between 15 g/I and 100 g/I, between 20 g/I and 100 g/I, or between 25 g/I and 100 g/l. In some embodiments, the concentration of produced RebM in the culture medium can be, for example, between 5 g/I and 100 g/I, e.g., between 5 g/I and 50 to 90 g/I, between 10 g/I and 80 g/I, between 10 g/I and 75 g/I, between 20 g/I and 80 g/I, or between 20 g/I and 80 g/l. In some embodiments, the RebM concentration can be greater than 5 g/I, e.g., greater than 8.5 g/I, greater than 12 g/I, greater than 15.5 g/I, greater than 19 g/I, greater than 22.5 g/I, greater than 26 g/I, greater than 29.5 g/I, greater than 33 g/I, or greater than 36.5 g/l. In some embodiments, concentrations of produced RebM can be 40 g/I or greater, e.g., 50 g/I, 60 g/I 70 g/I 80 g/I, 90 g/I e.g., or greater. For example, in some embodiments, concentrations of produced RebM in the culture medium can be 100 g/I or greater. In some embodiments, expression of a variant UDP glycosyltransferase polypeptide, e.g., the polypeptide of any one of SEQ ID NO: 2-30, enhances production of RebM, compared to a counterpart control strain that is not modified to express the UDP glycosyltransferase polypeptide, is enhanced by at least 5%, or at least 10%, 20%, 30%, 40%, 50%, 60%, 70%, 80%, 90%, 100%, or greater, compared to the control.

In some embodiments, the host cells (e.g., yeast cells) produce RebA. The concentration of produced RebA in the culture medium can be, for example, between 1 g/I and 125 g/I, e.g., between 5 g/I and 115 g/I, between 10 g/I and 110 g/I, between 15 g/I and 100 g/I, between 20 g/I and 100 g/I, or between 25 g/I and 100 g/l. In some embodiments, the concentration of produced RebA in the culture medium can be, for example, between 5 g/I and 100 g/I, e.g., between 5 g/I and 50 to 90 g/I, between 10 g/I and 80 g/I, between 10 g/I and 75 g/I, between 20 g/I and 80 g/I, or between 20 g/I and 80 g/l.

In some embodiments, the RebA concentration can be greater than 5 g/I, e.g., greater than 8.5 g/I, greater than 12 g/I, greater than 15.5 g/I, greater than 19 g/I, greater than 22.5 g/I, greater than 26 g/I, greater than 29.5 g/I, greater than 33 g/I, or greater than 36.5 g/l. In some embodiments, concentrations of produced RebA can be 40 g/I or greater, e.g., 50 g/I, 60 g/I 70 g/I 80 g/I, 90 g/I e.g., or greater. For example, in some embodiments, concentrations of produced RebA in the culture medium can be 100 g/I or greater. In some embodiments, expression of a variant UDP glycosyltransferase polypeptide, e.g., the polypeptide of any one of SEQ ID NO: 2-30, enhances production of RebA, compared to a counterpart control strain that is not modified to express the UDP glycosyltransferase polypeptide, is enhanced by at least 5%, or at least 10%, 20%, 30%, 40%, 50%, 60%, 70%, 80%, 90%, 100%, or greater, compared to the control.

In some embodiments, the host cells (e.g., yeast cells) produce RebB. The concentration of produced RebB in the culture medium can be, for example, between 1 g/I and 125 g/I, e.g., between 5 g/I and 115 g/I, between 10 g/I and 110 g/I, between 15 g/I and 100 g/I, between 20 g/I and 100 g/I, or between 25 g/I and 100 g/l. In some embodiments, the concentration of produced RebB in the culture medium can be, for example, between 5 g/I and 100 g/I, e.g., between 5 g/I and 50 to 90 g/I, between 10 g/I and 80 g/I, between 10 g/I and 75 g/I, between 20 g/I and 80 g/I, or between 20 g/I and 80 g/l. In some embodiments, the RebB concentration can be greater than 5 g/I, e.g., greater than 8.5 g/I, greater than 12 g/I, greater than 15.5 g/I, greater than 19 g/I, greater than 22.5 g/I, greater than 26 g/I, greater than 29.5 g/I, greater than 33 g/I, or greater than 36.5 g/l. In some embodiments, concentrations of produced RebB can be 40 g/I or greater, e.g., 50 g/I, 60 g/I 70 g/I 80 g/I, 90 g/I e.g., or greater. For example, in some embodiments, concentrations of produced RebB in the culture medium can be 100 g/I or greater. In some embodiments, expression of a variant UDP glycosyltransferase polypeptide, e.g., the polypeptide of any one of SEQ ID NO: 2-30, enhances production of RebB, compared to a counterpart control strain that is not modified to express the UDP glycosyltransferase polypeptide, is enhanced by at least 5%, or at least 10%, 20%, 30%, 40%, 50%, 60%, 70%, 80%, 90%, 100%, or greater, compared to the control.

In some embodiments, the host cells (e.g., yeast cells) produce RebD. The concentration of produced RebD in the culture medium can be, for example, between 1 g/I and 125 g/I, e.g., between 5 g/I and 115 g/I, between 10 g/I and 110 g/I, between 15 g/I and 100 g/I, between 20 g/I and 100 g/I, or between 25 g/I and 100 g/l. In some embodiments, the concentration of produced RebD in the culture medium can be, for example, between 5 g/I and 100 g/I, e.g., between 5 g/I and 50 to 90 g/I, between 10 g/I and 80 g/I, between 10 g/I and 75 g/I, between 20 g/I and 80 g/I, or between 20 g/I and 80 g/l. In some embodiments, the RebD concentration can be greater than 5 g/I, e.g., greater than 8.5 g/I, greater than 12 g/I, greater than 15.5 g/I, greater than 19 g/I, greater than 22.5 g/I, greater than 26 g/I, greater than 29.5 g/I, greater than 33 g/I, or greater than 36.5 g/l. In some embodiments, concentrations of produced RebD can be 40 g/I or greater, e.g., 50 g/I, 60 g/I 70 g/I 80 g/I, 90 g/I e.g., or greater. For example, in some embodiments, concentrations of produced RebD in the culture medium can be 100 g/I or greater. In some embodiments, expression of a variant UDP glycosyltransferase polypeptide, e.g., the polypeptide of any one of SEQ ID NO: 2-30, enhances production of RebD, compared to a counterpart control strain that is not modified to express the UDP glycosyltransferase polypeptide, is enhanced by at least 5%, or at least 10%, 20%, 30%, 40%, 50%, 60%, 70%, 80%, 90%, 100%, or greater, compared to the control.

In some embodiments, the host cells (e.g., yeast cells) produce RebE. The concentration of produced RebE in the culture medium can be, for example, between 1 g/I and 125 g/I, e.g., between 5 g/I and 115 g/I, between 10 g/I and 110 g/I, between 15 g/I and 100 g/I, between 20 g/I and 100 g/I, or between 25 g/I and 100 g/l. In some embodiments, the concentration of produced RebE in the culture medium can be, for example, between 5 g/I and 100 g/I, e.g., between 5 g/I and 50 to 90 g/I, between 10 g/I and 80 g/I, between 10 g/I and 75 g/I, between 20 g/I and 80 g/I, or between 20 g/I and 80 g/l.

In some embodiments, the RebE concentration can be greater than 5 g/I, e.g., greater than 8.5 g/I, greater than 12 g/I, greater than 15.5 g/I, greater than 19 g/I, greater than 22.5 g/I, greater than 26 g/I, greater than 29.5 g/I, greater than 33 g/I, or greater than 36.5 g/l. In some embodiments, concentrations of produced RebM can be 40 g/I or greater, e.g., 50 g/I, 60 g/I 70 g/I 80 g/I, 90 g/I e.g., or greater. For example, in some embodiments, concentrations of produced RebE in the culture medium can be 100 g/I or greater. In some embodiments, expression of a variant UDP glycosyltransferase polypeptide, e.g., the polypeptide of any one of SEQ ID NO: 2-30, enhances production of RebE, compared to a counterpart control strain that is not modified to express the UDP glycosyltransferase polypeptide, is enhanced by at least 5%, or at least 10%, 20%, 30%, 40%, 50%, 60%, 70%, 80%, 90%, 100%, or greater, compared to the control.

### Fermentation Compositions

Also provided are fermentation compositions including a population host cells. The host cells may be any of the host cells disclosed herein and discussed above. In some embodiments, the fermentation composition further includes at least one steviol glycoside (e.g., RebA, RebB, RebD, RebE, and RebM) produced by the host cell. The at least one steviol glycoside can include, for example, RebA, RebB, RebD, RebE, and RebM. In some embodiments, the steviol glycoside includes RebM.

In some embodiments, the fermentation composition includes at least two steviol glycosides produced from the host cells. In some embodiments, the fermentation composition includes at least three steviol glycosides produced from the host cells. In some embodiments, the fermentation composition includes at least four steviol glycosides produced from the host cells. In some embodiments, the fermentation composition includes at least five steviol glycosides produced from the host cells.

The mass fraction of RebM within the one or more produced steviol glycosides can be, for example, between 0 and 50%, e.g., between 0 and 30%, between 5% and 35%, between 10% and 40%, between 15% and 45%, or between 20% and 40%. In terms of upper limits, the mass fraction of RebM in the steviol glycosides can be less than 50%, e.g., less than 45%, less than 40%, less than 35%, less than 30%, less than 25%, less than 20%, less than 15%, less than 10%, or less than 5%.

### Methods of Recovering Steviol Glycosides

Also provided are methods of recovering one or more steviol glycosides (e.g., one or more of RebA, RebB, RebD, RebE, or RebM) from a fermentation composition. In some embodiments, the fermentation composition is any of the fermentation compositions disclosed herein and described above. The method may include separating at least a portion of a population of host cells from a culture medium. In some embodiments, the separating includes using centrifugation. In some embodiments, the separating includes using filtration.

While some portion of the one or more steviol glycosides (e.g., one or more of RebA, RebB, RebD, RebE, or RebM) produced by the cells during fermentation can be expected to partition with the culture medium during the separation of the host cells from the medium, some of the steviol glycosides can be expected to remain associated with the yeast cells. One approach to capturing this cell-associated product and improving overall recovery yields is to rinse the separated cells with a wash solution that is then collected.

The provided recovery methods further include contacting the separated yeast cells with a heated wash liquid. In some embodiments, the heated wash liquid is a heated aqueous wash liquid. In some embodiments, the heated wash liquid consists of water. In some embodiments, the heated wash liquid includes one or more other liquid or dissolved solid components.

The temperature of the heated aqueous wash liquid can be, for example, between 30° C. and 90° C., e.g., between 30° C. and 66° C., between 36° C. and 72° C., between 42° C. and 78° C., between 48° C. and 84° C., or between 54° C. and 90° C. In terms of upper limits, the wash temperature can be less than 90° C., e.g., less than 84° C., less than 78° C., less than 72° C., less than 66° C., less than 60° C., less than 54° C., less than 48° C., less than 42° C., or less than 36° C. In terms of lower limits, the wash temperature can be greater than 30° C., e.g., greater than 36° C., greater than 42° C., greater than 48° C., greater than 54° C., greater than 60° C., greater than 66° C., greater than 72° C., greater than 78° C., or greater than 84° C. Higher temperatures, e.g., greater than 90° C., and lower temperatures, e.g., less than 30° C., are also contemplated.

The method may further include, subsequent to the contacting of the separated host cells with the heated wash liquid, removing the wash liquid from the host cells. In some embodiments, the removed wash liquid is combined with the separated culture medium and further processed to isolate the one or more steviol glycosides (e.g., one or more of RebA, RebB, RebD, RebE, or RebM) that has been produced. In some embodiments, the removed wash liquid and the separated culture medium are further processed independently of one another. In some embodiments, the removal of the wash liquid from the host cells includes cetrifugation. In some embodiments, the removal of the wash liquid from the host cells includes filtration.

The recovery yield can be such that, for at least one of the one or steviol glycosides (e.g., one or more of RebA, RebB, RebD, RebE, or RebM) produced from the host cells, the mass fraction of the produced at least one steviol glycoside recovered in the combined culture medium and wash liquid is, for example, between 70% and 100%, e.g., between 70% and 88%, between 73% and 91%, between 76% and 94%, between 79% and 97%, or between 82% and 100%. In terms of lower limits, the recovery yield of at least one of the one or more steviol glycosides can be greater than 70%, e.g., greater than 73%, greater than 76%, greater than 79%, greater than 82%, greater than 85%, greater than 88%, greater than 91%, greater than 94%, or greater than 97%. The recovery yield can be such that, for each of the one or more steviol glycosides produced from the host cells, the mass fraction recovered in the combined culture medium and wash liquid is, for example, between 70% and 100%, e.g., between 70% and 88%, between 73% and 91%, between 76% and 94%, between 79% and 97%, or between 82% and 100%. In terms of lower limits, the recovery yield of each of the one or more steviol glycosides can be greater than 70%, e.g., greater than 73%, greater than 76%, greater than 79%, greater than 82%, greater than 85%, greater than 88%, greater than 91%, greater than 94%, or greater than 97%.

While the compositions and methods provided herein have been described with respect to a limited number of embodiments, one or more features from any of the embodiments described herein or in the figures can be combined with one or more features of any other embodiment described herein in the figures without departing from the scope of the disclosure. No single embodiment is representative of all aspects of the methods or compositions. In certain embodiments, the methods can include numerous steps not mentioned herein. In certain embodiments, the methods do not include any steps not enumerated herein. Variations and modifications from the described embodiments exist.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Yeast Transformation Methods

Each DNA construct was integrated into Saccharomyces cerevisiae (CEN.PK113-7D) using standard molecular biology techniques in an optimized lithium acetate transformation. Briefly, cells were grown overnight in yeast extract peptone dextrose (YPD) media at 28° C. with shaking (200 rpm), diluted to an OD600 of 0.1 in 100 mL YPD, and grown to an OD600 of 0.6-0.8. For each transformation, 5 mL of culture were harvested by centrifugation, washed in 5 mL of sterile water, spun down again, resuspended in 1 mL of 100 mM lithium acetate, and transferred to a microcentrifuge tube. Cells were spun down (13,000×g) for 30 s, the supernatant was removed, and the cells were resuspended in a transformation mix consisting of 240 μL 50% PEG, 36 μL 1 M lithium acetate, 10 μL boiled salmon sperm DNA, and 74 μL of donor DNA. For transformations that require expression of the endonuclease F-CphI, the donor DNA included a plasmid carrying the F-CphI gene expressed under the yeast TDH3 promoter. F-CphI endonuclease expressed in such a manner cuts a specific recognition site engineered in a host strain to facilitate integration of the target gene of interest. Following a heat shock at 42° C. for 40 min, cells were recovered overnight in YPD media before plating on selective media. DNA integration was confirmed by colony PCR with primers specific to the integrations.

### Example 2: Generation of a Base Strain Capable of High Flux to Farnesyl Pyrophosphate and the Isoprenoid Farnesene

A farnesene production strain was created from a wild-type Saccharomyces cerevisiae strain (CEN.PK113-7D) by expressing the genes of the MEV pathway under the control of native GAL promoters. This strain comprised the following chromosomally integrated mevalonate pathway genes from S. cerevisiae: acetyl-CoA thiolase, HMG-CoA synthase, HMG-CoA reductase, mevalonate kinase, phosphomevalonate kinase, mevalonate pyrophosphate decarboxylase, and IPP:DMAPP isomerase. In addition, the strain contained multiple copies of farnesene synthase from Artemisia annua, also under the control of either native GAL1 or GAL10 promoters. All heterologous genes described herein were codon optimized using publicly available or other suitable algorithms. The strain also contained a deletion of the GAL80 gene. Examples of methods for creating S. cerevisiae strains with high flux to isoprenoids are described in the U.S. Pat. Nos. 8,415,136 and 8,236,512 which are incorporated herein in their entireties.

### Example 3: Construction of a Series of Strains for Rapid Screening for Novel p-Glycosyltransferase Catalyzing the Transfer of a Glucose Moiety from Donor UDP-Glucose to the 2′ Position of the 13-O-Glucose of the Acceptor Molecules, Steviolmonoside or Rubusoside

The farnesene base strain described above was further engineered to have high flux to the C20 isoprenoid kaurene by integrating into the genome four copies of a geranylgeranyl pyrophosphate synthase (GGPPS), two copies of a copalyldiphosphate synthase, and one copy of a kaurene synthase. Subsequently, all copies of farnesene synthase were removed from the strain and the strain was confirmed to produce ent-kaurene and no farnesene.

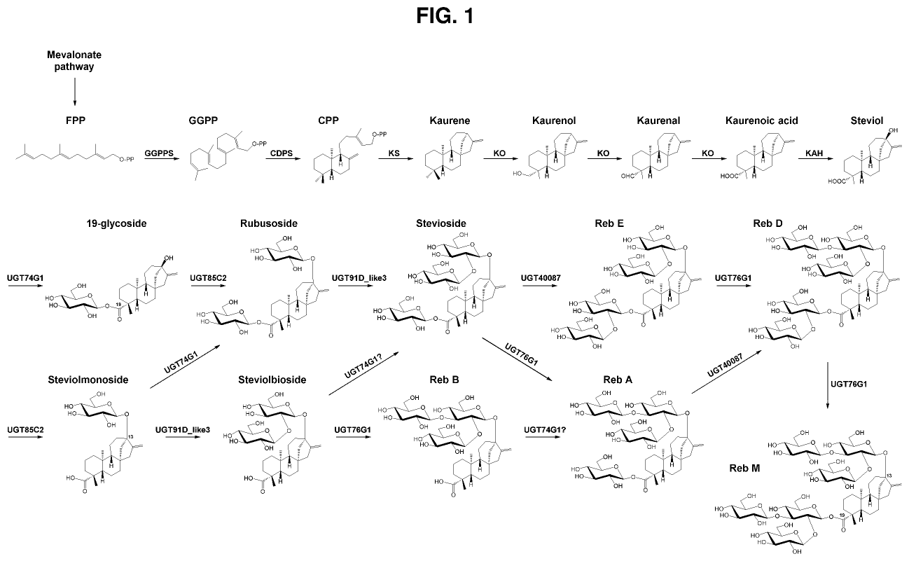

The conversion of ent-kaurene to RebM requires the activity of two cytochrome P450 enzymes (KO and KAH), accompanying reductase CPR, and five glycosyltransferases (FIG. 1). Table 3 lists all the genes and promoters used in yeast strains that produced RebM. Incorporation of the second of the three glucose moieties present at C13 position of RebM required a dedicated glycosyltransferase (UGT91 D_like3 in FIG. 1) to transfer a glucose moiety from donor UDP-D-glucose to the 2′ position of the 13-O-glucose of the acceptor molecules, where the acceptor can be either steviolmonoside or rubusoside.

To screen glycosyltransferases for UGT91 D_like3 activity in vivo in S. cerevisiae, a series of yeast host strains were generated that contained all the genes necessary for the biosynthesis of RebM, with the exception of any glycosyltransferase with the activity of UGT91 D_like3. The strains containing all genes described in Table 3 except UGT91 D_like3 primarily produce rubusoside, a product of sequential glycosylation of steviol by the action of glycosyltransferases UGT74G1 and UGT85C2. Rubusoside was the substrate for UGT91 D_like3 or homologous glycosyltransferase. When UGT91 D_like3 or enzyme with the same activity was integrated in these hosts, RebM is produced.

TABLE 3
Genes, promoters, and amino acid sequences
of the enzymes used to convert FPP to RebM.

| Enzyme | SEQ ID NO | Promoter |
|---|---|---|
| Bt.GGPPS | 41 | PGAL1 |
| Ent-Os.CDPS | 42* | PGAL1 |
| Ent-Pg.KS | 43 | PGAL1 |
| Ps.KO | 44 | PGAL1 |
| At.CPR | 45 | PGAL3 |
| Sr.KAH mutant #3 | 46 | PGAL1 |
| UGT85C2 | 36 | PGAL10 |
| UGT74G1 | 37 | PGAL1 |
| UGT91D_like3 | 38 | PGAL1 |
| UGT76G1 | 39 | PGAL10 |
| UGT40087 | 40 | PGAL1 |

*First 65 amino acids replaced with methionine.

In addition to the host strains described above, strains were also constructed that lacked not only UGT91 D_like3 but also glycosyltransferases UGT76G1 and UGT40087. These host strains also primarily produced rubusoside, a product of sequential glycosylation of steviol by UGT74G1 and UGT85C2. When UGT91 D_like3 or enzyme with the same activity was added to strains with partial RebM pathway, stevioside was produced as the major product and no RebM was formed (FIG. 1).

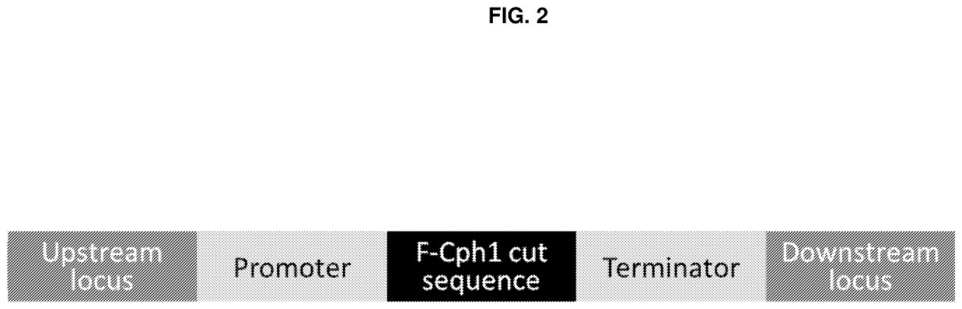

To measure the activity of enzymes with UGT91 D_like3 activity in vivo in S. cerevisiae, the hosts with complete or partial RebM pathway described above were engineered to contain a landing pad to allow for the rapid insertion of genes encoding UGT91 D_like3 homologs and variants (FIG. 2). The landing pad consisted of 500 bp of locus-targeting DNA sequences on either end of the construct to the genomic region upstream and downstream of the yeast locus of choice (Upstream locus and Downstream locus), thereby deleting the locus when the landing pad was integrated into the yeast chromosome. Internally, the landing pad contained a promoter (Promoter) which could be GAL1, GAL3 or any other promoter of yeast GAL regulon and a yeast terminator of choice (Terminator) flanking an endonuclease recognition site (F-CphI). DNA of UGT91 D_like3 homologs and variants with flanking sequences homologous to promoters and terminators of the landing pads were used to transform the strain along with a plasmid expressing endonuclease F-CphI, which cut the recognition sequence, creating a double strand break at the landing pad, and facilitating homologous recombination of the UGT gene DNA at the site.

A series of yeast strains were constructed as described above with landing pads that contained either a GAL1 or a GAL3 promoter. The strong GAL1 promoter allowed for the highest expression of the gene integrated immediately downstream thus allowing for detection of even weak glycosyltransferase activity. However, different highly active glycosyltransferase variants may not be distinguishable when expressed under GAL1 promoter, e.g., if the substrate for glycosyltransferase of interest becomes limiting. Thus, hosts containing landing pads with the significantly weaker GAL3 promoter were used in some of the experiments with highly active target glycosyltransferases.

### Example 4: Yeast Culturing Conditions

Yeast colonies verified to contain the expected glycosyltransferase gene were picked into 96-well microtiter plates containing Bird Seed Media (BSM, originally described by van Hoek et al., Biotechnology and Bioengineering 68(5), 2000, pp. 517-523) with 14 g/L sucrose, 7 g/L maltose, 37.5 g/L ammonium sulfate, and 1 g/L lysine. Cells were cultured at 28° C. in a high-capacity microtiter plate incubator shaking at 1000 rpm and 80% humidity for 3 days until the cultures reached carbon exhaustion. The growth-saturated cultures were subcultured into fresh plates containing BSM with 40 g/L sucrose, 37.5 g/L ammonium sulfate, and 1 g/L lysine by taking 14.4 μL from the saturated cultures and diluting into 360 μL of fresh media. Cells in the production media were cultured at 30° C. in a high-capacity microtiter plate shaker at 1000 rpm and 80% humidity for additional 3 days prior to extraction and analysis.

### Example 5: Yeast Sample Preparation Conditions for Analysis of Pathway Intermediates from Farnesol to Rebaudioside M

To extract all steviol glycosides made by cells (see FIG. 1), upon culturing completion, the whole cell broth was diluted with 628 μL of 100% ethanol, sealed with a foil seal, and shaken at 1250 rpm for 30 s. 314 μL of water was added to each well directly to dilute the extraction. The plate was briefly centrifuged to pellet solids. 198 μL of 50:50 ethanol:water containing 0.48 mg/L rebaudioside N, used as an internal standard, was transferred to a new 250 μL assay plate and 2 μL of the culture/ethanol mixture was added to the assay plate. A foil seal was applied to the plate for analysis. The samples were analyzed using either high throughput mass spectrometry assay or lower throughput liquid chromatography-mass spectrometry assay.

### Example 6: Analytical Methods

The samples derived from yeast producing steviol glycosides (Example 5) were routinely analyzed using mass spectrometer (Agilent 6470-QQQ) with a RapidFire 365 system autosampler with C8 cartridge using the parameters described in Tables 4 and 5. Steviol glycosides were measured in the assay.

TABLE 4
RapidFire 365 system configuration.

| | |
|---|---|
| Pump 1, Line A: 2 mM ammonium formate in | 100% A, 1.5 mL/min |
| water | |
| Pump 2, Line A: 35% acetonitrile in water | 100% A, 1.5 mL/min |
| Pump 3, Line A: 80% acetonitrile in water | 100% A, 0.8 mL/min |
| State 1: Aspirate | 600 ms |
| State 2: Load/wash | 3000 ms |
| State 3: Extra wash | 1500 ms |
| State 4: Elute | 5000 ms |
| State 5: Reequilibrate | 1000 ms |

TABLE 5
6470-QQQ MS method configuration.

| Ion source | AJS ESI | |
|---|---|---|
| Time filtering peak width | 0.02 | min |
| Stop time | No limit/as pump | |
| Scan type | MRM | |
| Diverter valve | To MS | |
| Delta EMV | (+)0/(−)300 | |
| Ion mode (polarity) | Negative | |
| Gas temperature | 250° | C. |
| Gas flow | 11 | L/min |
| Nebulizer | 30 | psi |
| Sheath gas temperature | 350° | C. |
| Sheath gas flow | 11 | L/min |
| Negative capillary voltage | 2500 | V |

The mass spectrometer was operated in negative ion multiple reaction monitoring (MRM) mode. Each steviol glycoside was identified from precursor ion mass and MRM transition (Table 6). The fragmentation at labile carboxylic ester linkage at the C19 allowed for distinction between regioisomers RebA and RebE while no distinction can be made between rubusoside and steviolbioside (steviol+2Glc) or stevioside and RebB (steviol+3Glc) using this method.

TABLE 6
Steviol glycosides and masses for corresponding
precursor and product ions.

| Compound | Precursor ion (Da) | Product ion (Da) |
|---|---|---|
| steviol + 1Glc | 479.265 | 317.212 |
| steviol + 2Glc | 641.318 | 479.265 |
| steviol + 3Glc | 803.371 | 641.318 |
| RebA | 965.424 | 803.371 |
| RebE | 965.424 | 641.318 |
| steviol + 5Glc | 1127.476 | 803.371 |
| steviol + 6Glc | 1289.529 | 803.371 |

The peak areas from a chromatogram from a mass spectrometer were used to generate the calibration curve using authentic standards. The molar ratios of relevant compounds were determined by quantifying the amount in moles of each compound through external calibration using an authentic standard, and then taking the appropriate ratios.

To determine specific steviol glycosides and to evaluate the presence of new side products, selected samples were also analyzed using ultra-high-performance liquid chromatography (UHPLC) on Thermo Fisher Scientific Vanquish UHPLC system equipped with Acquity UPLC BEH C18 column (15 cm, 2.1 mm, 1.7 μm, 130 Å; part #186002353) (Table 7). Dual detection was performed using Vanquish charged aerosol detector (CAD) (Table 8) and Thermo Fisher Scientific Q-Exactive Orbitrap mass spectrometer (Table 9) with post-column flow split 5:1 (5 to CAD and 1 to MS) using Restek binary fixed-flow splitter.

TABLE 7
Vanquish UHPLC chromatographic conditions.

| Mobile phase A | 0.1% formic acid in water | | |
|---|---|---|---|
| Mobile phase B | 0.1% formic acid in acetonitrile | | |
| Flow rate | 0.4 mL/min | | |
| Column temperature | 50° C. | | |
| Pre-heater temperature | 50° C. | | |
| Gradient | Time (min) | A % | B % |
| | 0 | 80 | 20 |
| | 2 | 80 | 20 |
| | 28 | 54 | 46 |
| | 28.1 | 5 | 95 |
| | 32 | 5 | 95 |
| | 32.5 | 80 | 20 |
| | 36 | 80 | 20 |

TABLE 8
Vanquish CAD detector configuration.

| Power function | 1.00 | |
|---|---|---|
| Data collection rate | 2 | Hz |
| Filter | 3.6 | |
| Gas regulation mode | Analytical | |
| Evaporator temperature | 35° | C. |

TABLE 9
Q-Exactive Orbitrap MS method configuration.
Ion source conditions:

| Ion source | ESI | |
|---|---|---|
| Sheath gas flow rate | 40 | |
| Auxiliary gas flow rate | 15 | |
| Sweep gas flow rate | 2 | |
| Spray voltage | 3500 | V |
| Capillary temperature | 375° | C. |
| S-Lens RF level | 60.0 | |
| Auxiliary gas heater temperature | 400° | C. |

Scan settings:

| | | | |
|---|---|---|---|
| General | Runtime | 0 to 36 | min |

| | | |
|---|---|---|
| | Polarity | Negative |
| | Default charge state | 1 |
| | Inclusion | On |
| | Exclusion | On |
| | Scan type | Full MS − ddMS² |
| Full MS | Resolution | 70,000 |
| | AGC target | 1e6 |

| | | |
|---|---|---|
| Maximum IT | 50 | ms |
| Scan range | 300 to 2000 | m/z |

| | | |
|---|---|---|
| | Spectrum data type | Centroid |
| ddMS² | Resolution | 35,000 |
| | AGC target | 1e5 |

| Maximum IT | 50 | ms |
|---|---|---|
| Loop count | 10 | |
| TopN | 10 | |
| Isolation window | 2.0 | m/z |

| | | |
|---|---|---|
| | Stepped (N)CE | nce: 10, 30, 40 |
| dd Settings | Minimum AGC target | 8.00e3 |
| | Charge exclusion | >3 |
| | Exclude isotopes | On |

| | | |
|---|---|---|
| Dynamic exclusion | 4.0 | s |

| | |
|---|---|
| If idle . . . | Pick others |

The mass spectrometer was operated in negative ion multiple reaction monitoring mode. The peak identities were assigned to steviol glycosides based on retention time determined from an authentic standard, molecular ion, and MRM transition (Table 10).

TABLE 10
Steviol glycosides, their retention times and precursor ion.

| Compound | Retention time (min) | Precursor ion (Da) |
|---|---|---|
| Steviol | 27.8 | 317.212 |
| Steviolmonoside | 20.6 | 479.265 |
| 19-glycoside | 19.4 | 479.265 |
| Steviolbioside | 17.5 | 641.318 |
| Rubusoside | 15.5 | 641.318 |
| RebB | 17.6 | 803.371 |
| Stevioside | 12.7 | 803.371 |
| RebE | 7.4 | 965.424 |
| RebA | 12.7 | 965.424 |
| RebD | 8.0 | 1127.476 |
| RebM | 8.8 | 1289.529 |

### Example 7: Novel p-Glycosyltransferase Ob.UGT91B1 Identified Via Activity Screen of Diverse Glycosyltransferases Efficiently Catalyzes the Transfer of a Glucose Moiety from Donor UDP-Glucose to the 2′ Position of the 13-O-Glucose of the Acceptor Molecules in RebM Biosynthetic Pathway

Previously identified protein sequence Sr.UGT91 D_like3 (SEQ ID NO: 38) from the plant Stevia rebaudiana was used as a query to search for homologous glycosyltransferases in public databases using a variety of search algorithms: UniProt (https://www.uniprot.org), NCBI (https://blast.ncbi.nlm.nih.gov/Blast.cgi), HMMER (http://hmmer.org), Phytozome (the Plant Comparative Genomics portal of the Department of Energy's Joint Genome Institute; https://phytozome.jgi.doe.gov), Genome Database for Rosaceae (https://www.rosaceae.org). A collection of protein sequences was assembled and prioritized for analysis using CD-HIT clustering program (http://weizhongli-lab.org/cd-hit). Ultimately over 300 glycosyltransferase genes were integrated in the PGAL1 landing pad of yeast host containing RebM pathway (but lacking UGT91 D or any homologs). The resulting yeast strains were grown and analyzed for the production of RebM and other steviol glycosides as described above (Examples 4-6).

In addition to mass spectrometry-based high throughput assay, the identity of RebM produced by active glycosyltransferases was confirmed by comparison to RebM authentic standard in LC-CAD-MS assay with extended solvent gradient. The final product was indistinguishable from the standard in both retention time and mass spectrum supporting not only the composition of the final product as hexaglycosylated steviol but also the regio and stereo configurations of sugar linkages as those present in RebM.

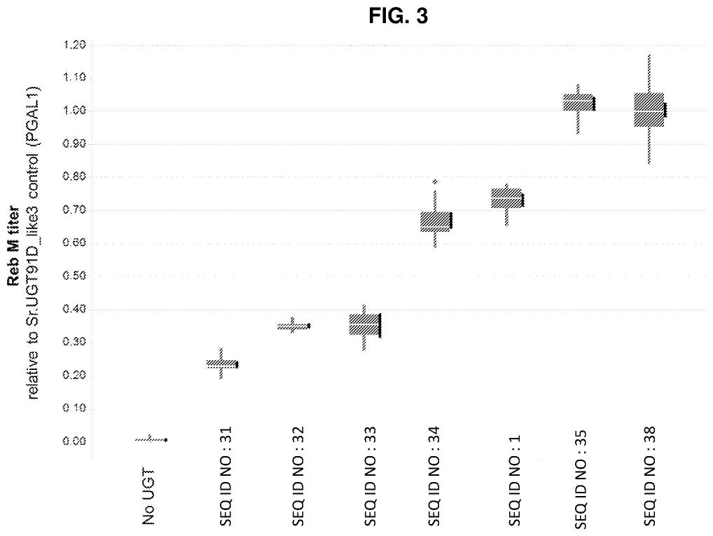

A total of six enzymes in addition to Sr.UGT91 D_like3 were identified that provided enzymatic activity necessary for RebM biosynthesis, namely glycosylation at the 2′ position of the 13-O-glucose of the acceptor molecules steviolmonoside or rubusoside, also called UGT91 D activity (FIG. 3). The production of RebM was used to evaluate the activity of these glycosyltransferases relative to Sr.UGT91 D_like3 (Table 11).

TABLE 11
Glycosyltransferases with Sr.UGT91D_like3 activity identified from diversity screen (gene
variants expressed under pGAL1), their RebM titer relative to Sr.UGT91D_like3 (averaged over
16 replicas), standard deviation from the mean value, and % identity to Sr.UGT91D_like3.

| | | | | Standard | |
|---|---|---|---|---|---|
| | Protein | SEQ | Average RebM | deviation | |
| | accession | ID | titer relative to | from the | % Identity to |
| Glycosyltransferase | number | NO | Sr.UGT91D_like3 | mean | Sr.UGT91D_like3 |
| None | — | — | 0.00 | 0.01 | — |
| Ob.UGT91B1_like | XP_006650455.1 | 31 | 0.23 | 0.02 | 39 |
| Hv.UGT_v1 | BAJ94055.1 | 32 | 0.35 | 0.02 | 38 |
| EUGT11 | XP_015629141.1 | 33 | 0.35 | 0.05 | 39 |
| (Os.UGT91C1) | | | | | |
| Op.UGTx5_2 | A0A0E0KHX5 | 34 | 0.67 | 0.06 | 40 |
| Ob.UGT91B1 | XP_006650454.1 | 1 | 0.73 | 0.04 | 38 |
| Sr.UGT91D2 | B3VI56.1 | 35 | 1.02 | 0.04 | 97 |
| Sr.UGT91D_like3 | SEQ ID NO: 38 | 38 | 1 | 0.08 | 100 |

The most active new enzyme identified in this experiment, Sr.UGT91 D2, is also the closest homolog to Sr.UGT91 D_like3. Two other highly active glycosyltransferases identified are Ob.UGT91 B1 and Op.UGTx5_2. Interestingly, while glycosyltransferase Ob.UGT91 B1 was approximately 73% as active as Sr.UGT91 D_like3 in this particular host the proteins share only 38% amino acid sequence identity. Ob.UGT91 B1 is more similar (approximately 60% amino acid identity) to EUGT11 that is known to catalyze the same reaction of a 2′ glycosylation of the 13-O-glucosylated acceptor as a promiscuous side activity in addition to 2′ glycosylation of the 19-O-glucosylated acceptor as described in U.S. Pat. No. 11,091,743, which is incorporated herein by reference in its entirety.

### Example 8: Glycosyltransferase Ob.UGT91B1 Acts on 2′ Position not Only of 13-O-Glucose but Also of 19-O-Glucose in Steviol Glycoside Acceptors Forming RebE, Undesirable Glycosylation of RebE is Minor

As outlined in Example 7 several glycosyltransferases with UGT91 D activity, namely glycosylation at 2′ position of 13-O-glucose in steviol glycosides, were identified when candidates were screened in the context of full RebM pathway. To explore possible side-activities of these glycosyltransferases, each of the corresponding genes was integrated in the host strain that contained all of the genes needed for the biosynthesis of RebM except those encoding glycosyltransferases UGT76G1, UGT40087, and UGT91 D. Having only UGT74G1 and UGT85C2 of the pathway; this host produced rubusoside as the major product and steviolmonoside and 19-glycoside as the minor steviol glycoside products. Integration of any gene encoding UGT91 D activity in this host strain is expected to result in the formation of stevioside as a product of sequential glycosylation of steviol by UGT74G1, UGT85C2, and UGT91D (FIG. 1).

Seven genes encoding the proteins listed in Table 9 were integrated in the PGAL1 landing pad of yeast host containing partial RebM pathway, which lacked genes for UGT76G1, UGT40087, and UGT91 D). The resulting yeast strains were grown and analyzed for the production of steviol glycosides as described above (Examples 4-6). Mass spectrometry-based high throughput assay was used for initial characterization followed by a lower throughput LC-CAD-MS assay that allowed for structural characterization of steviol glycosides.

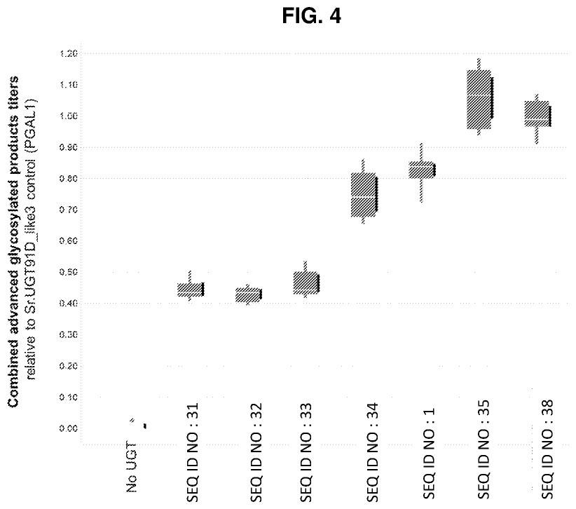

All of the strains described above produced not only expected product stevioside (contains three glucose moieties) but also other advanced glycosylated products containing four or five glucose moieties. The combined titers of glycosylated products with three, four, and five glucose moieties produced in the presence of glycosyltransferase enzymes relative to those produced by Sr.UGT91 D_like3 (FIG. 4) ranked the enzymes roughly the same as in the strains with full RebM pathway (FIG. 3)—Sr.UGT91 D_like3 and Sr.UGT91 D2 were most active, followed by Ob.UGT91 B1 and Op.UGTx5_2, and then by the rest.

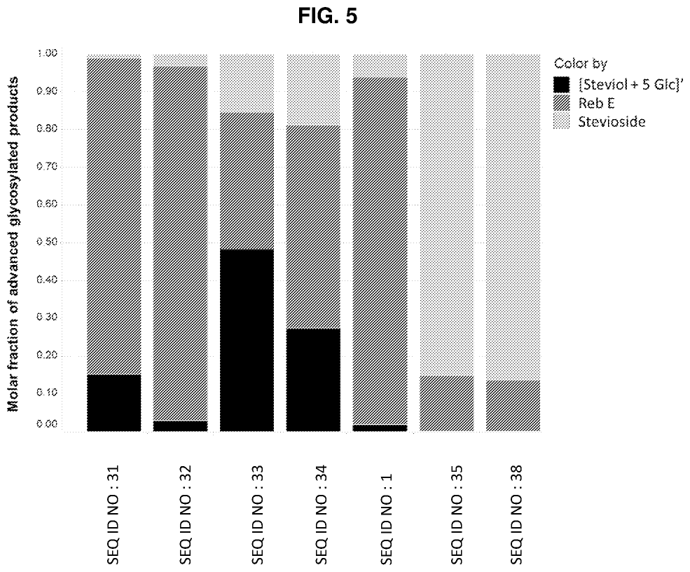

The composition of advanced glycosylated products was different for different enzymes suggesting differing substrate and/or product preferences (FIG. 5). Stevioside was identified as the major product produced by yeast strains harboring Sr.UGT91 D_like3 or Sr.UGT91 D2. In addition to stevioside these strains also produced minor quantities of RebE. Formation of RebE indicates that these glycosyltransferases can accept stevioside as the substrate glycosylating it at 2′ position of 19-O-glucose, UGT40087-like activity. The ability of these glycosyltransferases to convert RebA to RebD, also UGT40087-like activity, has been previously documented in U.S. Pat. No. 11,091,743, which is incorporated herein by reference in its entirety. Conversion of stevioside to RebE has been shown for EUGT11 (Zhang J, Tang M, Chen Y, Ke D, Zhou J, Xu X, Yang W, He J, Dong H, Wei Y, Naismith JH, Lin Y, Zhu X, Cheng W. Nat. Commun. 2021, 12, 7030).

RebE was the major product for the glycosyltransferases Ob.UGT91 B1, Ob.UGT91 B1_like, Hv.UGT_v1, and Op.UGTx5_2 indicating even higher UGT40087-like activity towards stevioside. In addition to RebE these promiscuous enzymes also generated a significant fraction of steviol glycoside product containing five glucose moieties ([Steviol+5 Glc]′ in FIG. 5). [Steviol+5 Glc]′ was the major product produced in the presence of EUGT11 with remaining products being RebE and stevioside.

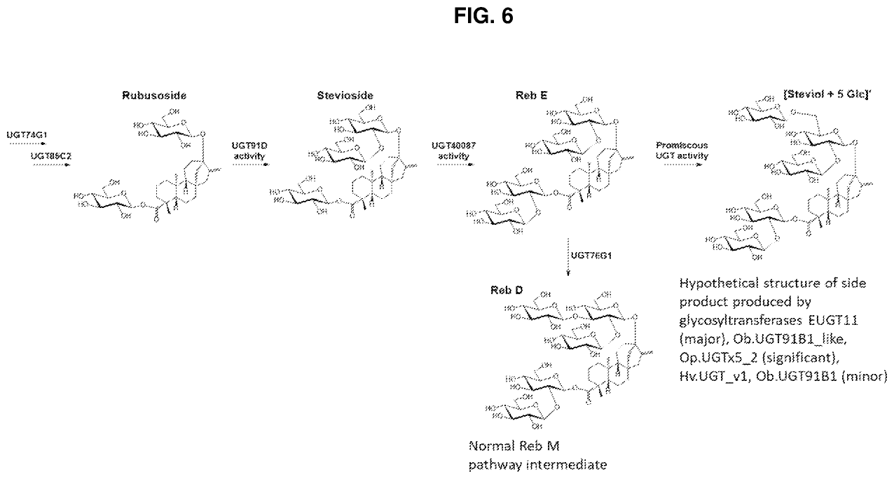

Initial mass spectrometry-based high throughput analysis suggested that [Steviol+5 Glc]′ might have a structure of RebD, a normal RebM pathway intermediate: a major ion of 803.371 Da was formed from a parent ion of 1127.476 Da indicating that a chain of two glucose moieties is located at more labile C19 position of steviol, and a chain of three glucose moieties was a substituent at C13 of steviol (as in RebD). This is highly surprising as the presence of UGT76G1 is necessary for the formation of RebD (FIG. 1). However, analysis using LC-CAD-MS assay and comparison to authentic standard of RebD clearly confirmed that [Steviol+5 Glc]′ did not have the structure of RebD. It must therefore have different connectivity of glucose moieties, for example as depicted in FIG. 6. Although not confirmed with authentic standard or NMR, the structure for [Steviol+5 Glc]′ depicted in FIG. 6 was supported by the recent publication describing this particular product (referred to as RebE-X) as the result of glycosyltransferase EUGT11 (referred to as OsUGT91 Cl) acting on RebE (Zhang J, Tang M, Chen Y, Ke D, Zhou J, Xu X, Yang W, He J, Dong H, Wei Y, Naismith JH, Lin Y, Zhu X, Cheng W. Nat. Commun. 2021, 12, 7030).

FIG. 6 summarizes the proposed reactions catalyzed by seven glycosyltransferases tested in this example. All of the enzymes are proficient in converting rubusoside to stevioside (UGT91 D activity) and in converting stevioside to RebE (UGT40087 activity) to different extents. Stevioside and RebE are intermediates found in RebM pathway. A subset of the enzymes was also able to further glycosylate RebE to form [Steviol+5 Glc]′ which is a side product that is not part of RebM pathway. Such activity is highly undesirable in yeast strains for RebM production as it diverts pathway intermediates away from RebM, diminishing its production at the very least and possibly having adverse effects on cell health.

Considering overall in vivo efficiency of the enzymes and their tendency to produce undesirable side product, e.g., [Steviol+5 Glc]′, Ob.UGT91 B1 was identified as one of the most promising candidates. While Ob.UGT91 B1 is highly active towards rubusoside and stevioside, it only produces minor quantities of [Steviol+5 Glc]′.

### Example 9: Evolution of Wild-Type Ob.UGT91B1 Via Site-Directed Saturation Mutagenesis

In this example, activity data is provided for wild-type Ob.UGT91 B1 and specific mutations of Ob.UGT91 B1 polypeptide sequence that led to improved production of steviol glycosides including RebM when expressed in S. cerevisiae host.

Each amino acid residue in Ob.UGT91 B1 (463 total, amino acid residues 2-464) was mutated using degenerate codon NNT, where N stands for any nucleotide adenine, thymine, guanine, and cytosine; and T stands for thymine. The degenerate codon NNT encoded 15 different amino acids (A, C, D, F, G, H, I, L, N, P, R, S [encoded by two codons], T, V, and Y). The library at each amino acid position was constructed via PCR using primers designed to introduce a degenerate codon so that each PCR product contains a mixture of gene variants where 15 possible different amino acids were encoded at a specific position corresponding to a single protein residue. In each PCR product, the pool of Ob.UGT91 B1 gene variants were flanked at 5′ end by 235 bp of sequence homologous to promoter (pGAL1) and at 3′ end by 238 bp of sequence homologous to terminator (tDIT1), both regions were part of the landing pad in a host strain as described in Example 3.

Each variant pool represented changes at a single amino acid position in Ob.UGT91 B1 and was used to independently transform a host yeast that contained all the genes necessary for the formation of RebM except for Sr.UGT91 D_like3 or other enzyme with such activity. For Tier 1 screening, 26 colonies were chosen per site to screen, roughly representing a 1.6× sampling coverage of the library. Every amino acid in the wild-type Ob.UGT91 B1 sequence (SEQ ID NO: 1) was subjected to mutagenesis and screening as described. The library was propagated as described in Example 4 and microtiter plate cultures were prepared and analyzed for the production of steviol glycosides including RebM as described in Examples 5 and 6 using mass spectrometry-based high throughput assay.

The effect of a particular mutation on Ob.UGT91 B1 activity was inferred by comparing RebM titer produced by a strain containing the mutant protein to RebM produced by a strain containing the wild-type Ob.UGT91 B1 protein. This ensured that improvements in desirable activity towards RebM formation were captured while improvements in undesirable side activity towards [Steviol+5 Glc]′ are ignored.

Upon finding mutations in Ob.UGT91 B1 that increased activity of the enzyme in vivo, a Tier 2 screen was performed with higher replication (n=8) to confirm the improvement in RebM production. The library hits confirmed in Tier 2 screen were subjected to confirmation in Tier 3 where nucleotide sequences of Tier 2 hits were PCR-amplified and cloned in a host yeast that had all the same feature as the host used in Tier 1 except the nucleotide sequences of Tier 2 hits were placed under the control of pGAL3, a promoter that was approximately 10 times weaker than pGAL1 used in the Tier 1 screen. As noted in Example 3, using a promoter of lower strength for validation of improved glycosyltransferase variants ensured that they remained limiting and thus distinguishable in the screen, instead of the screen being limited by supply of a substrate.

In total, 19 unique mutations that improved Ob.UGT91 B1 activity between 26% and 3.2-fold over wild type protein sequence were found by screening the libraries described above (Table 12). Table 12 lists the average fold improvement for each mutation over wild-type Ob.UGT91B1. The activity of wild-type Sr.UGT91 D_like3 is included for reference.

TABLE 12
Ob.UGT91B1 alleles that increase activity of wild-type
Ob.UGT91B1 measured as RebM produced in Tier 3 screen
(gene variants expressed under pGAL3). Associated amino
acid change, fold improvement in RebM production over
wild-type Ob.UGT91B1 (averaged over 4-8 replicas), and
standard deviation from the mean are listed.

| Ob.UGT91B1 | Fold improvement | Standard |
|---|---|---|
| sequence | over wild- | deviation |
| variation | type Ob.UGT91B1 | from the mean |
| wild-type Ob.UGT91B1 | 1.00 | 0.11 |
| R9S | 1.26 | 0.03 |
| P65S | 1.26 | 0.14 |
| S363N | 1.32 | 0.25 |
| R94N | 1.34 | 0.17 |
| V110S | 1.38 | 0.09 |
| D404T | 1.48 | 0.20 |
| G385I | 1.50 | 0.12 |
| R389F | 1.54 | 0.17 |
| D195A | 1.59 | 0.11 |
| G385H | 1.63 | 0.10 |
| R187P | 2.00 | 0.40 |
| D404S | 2.09 | 0.23 |
| R389N | 2.20 | 0.12 |
| V66R | 2.24 | 0.23 |
| R389H | 2.27 | 0.15 |
| V66F | 2.31 | 0.26 |
| R389D | 2.79 | 0.14 |
| L201N | 3.18 | 0.60 |
| G4N | 3.19 | 0.30 |
| Sr.UGT91D1_like3 | 5.12 | 0.35 |

### Example 10: Evolution of Ob.UGT91B1 Via Combinatorial Mutagenesis (12 Amino Acid Residues Targeted for Mutagenesis in a Full-Factorial Fashion)

A set of 12 mutations were selected from the unique site-directed saturation mutagenesis hits described in Example 9 to build a combinatorial library containing mutations G4N, R9S, P65S, V66F, R94N, V110S, R187P, D195A, L201N, G385H, R389D, D404T. The library was designed to create all possible combinations among the 12 mutations to find the combination that led to the highest activity of Ob.UGT91 B1 in vivo.

The genes were assembled from a mixture of PCR-amplified fragments containing desired mutations. Each fragment contained overlapping homology on the ends of each piece so that the pieces overlapped in sequence; assembling all the pieces together in vitro using PCR reconstituted a full-length Ob.UGT91 B1 allele. The terminal 5′ and 3′ pieces also had homology to the promoter and terminator of the landing pad sequence, which were pGAL3 and tDIT1 in this case, in RebM producing yeast that lacked a functional gene with UGT91 D activity. The assembled full-length library genes were transformed into yeast.

The Tier 1 combinatorial library DNA was screened in the RebM producing yeast at approximately 1.3× coverage. The effect of each mutation combination was calculated by comparing RebM produced by a strain containing the mutation combination to RebM produced by a strain containing the wild-type Ob.UGT91 B1 protein as described above (Example 9). The mutants that improved RebM production in Tier 1 screen were confirmed in Tier 2 and Tier 3; in this example, pGAL3 was used to drive mutant genes as in Tier 1, as described in Example 9.

The performance and associated amino acid changes for ten Ob.UGT91 B1 combinatorial mutagenesis hits promoted to Tier 3 are listed in Table 13. These variants contained from 5 to 9 amino acid mutations and produced at least 3-fold higher RebM as compared to wild-type Ob.UGT91 B1. Top hit, mutant #11, contained 7 mutations and produced 5.3-fold higher RebM in comparison to the wild-type Ob.UGT91 B1, which approached RebM titers produced by Sr.UGT91 D_like3 (5.8-fold higher than wild-type Ob.UGT91 B1). All improved variants contained amino acid changes L201N and R389D; both of these performed among top three mutations in site-directed saturation mutagenesis screen (Example 9, Table 12). The third top single amino acid change, G4N, also appeared among top combinatorial hits, but apparently the effect was not additive with L201N and R389D.

TABLE 13
Improved alleles of Ob.UGT91B1, fold improvement in RebM over
wild-type Ob.UGT91B1 activity, and the associated amino acid
changes. Combinatorial library hits were selected based on RebM
titers (averaged over 9 replicas) produced in Tier 3 screen.

| | Fold | |
|---|---|---|
| | improvement | |
| | over | |
| Ob.UGT91B1 | wild-type | |
| allele | Ob.UGT91B1 | Genotype of the mutant |
| wild-type | 1.00 | — |
| Ob.UGT91B1 | | |
| mutant #6 | 3.94 | P65S, V66F, V110S, R187P, |
| | | D195A, L201N, G385H, |
| | | R389D, D404T |
| mutant #7 | 4.03 | R9S, P65S, V110S, R187P, |
| | | L201N, R389D |
| mutant #5 | 4.17 | P65S, V110S, R187P, L201N, |
| | | G385H, R389D, D404T |
| mutant #3 | 4.21 | G4N, R94N, D195A, L201N, |
| | | G385H, R389D |
| mutant #2 | 4.38 | G4N, R94N, R187P, D195A, |
| | | L201N, R389D, D404T |
| mutant #8 | 4.51 | R94N, R187P, L201N, R389D, |
| | | D404T |
| mutant #10 | 4.59 | G4N, V16F, R94N, V110S, |
| | | L201N, R389D |
| mutant #9 | 4.85 | G4N, R9S, P65S, R187P, |
| | | D195A, L201N, R389D, |
| | | D404T |
| mutant #4 | 4.93 | R9S, R94N, D195A, L201N, |
| | | G385H, R389D, D404T |
| mutant #11 | 5.26 | P65S, R94N, V110S, D195A, |
| | | L201N, G385H, R389D |
| Sr.UGT91D_like3 | 5.81 | — |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

Sequence Appendix

SEQ ID NO: 1 Ob_UGT91B1
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 2 R9S
MASGRSSASAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 3 P65S
MASGRSSARAAGMMHVVICPWLAFGHLLPCLDLAQRLASRGHRVSFVSTPRNLSRLPPVRPALASV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 4 S363N
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWNSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 5 R94N
MASGRSSARAAGMMHVVICPWLAFGHLLPCLDLAQRLASRGHRVSFVSTPRNLSRLPPVRPALAPV
VSFVALPLPRVEGLPDGAESTNDVPQDNPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 6 V110S
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGSSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 7 D404T
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NTGDGSFDREGVAAVVRAVAVAEESRRVFRANAKKLQEIVADMACHDGYIDGFIQQLKSYKD
SEQ ID NO: 8 G385I
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQIPNARLIQAKKAGLQVPRN
DGDGSFDREGVAAVVRAVAVAEESRRVFRANAKKLQEIVADMACHDGYIDGFIQQLKSYKD
SEQ ID NO: 9 R389F
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNAFLIQAKKAGLQVPR
NDGDGSFDREGVAAVVRAVAVAEESRRVFRANAKKLQEIVADMACHDGYIDGFIQQLKSYKD
SEQ ID NO: 10 D195A
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKASSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 11 G385H
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNARLIQAKKAGLQVPR
NDGDGSFDREGVAAVVRAVAVAEESRRVFRANAKKLQEIVADMACHDGYIDGFIQQLKSYKD
SEQ ID NO: 12 R187P
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAAPRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 13 D404S
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NSGDGSFDREGVAAVVRAVAVAEESRRVFRANAKKLQEIVADMACHDGYIDGFIQQLKSYKD
SEQ ID NO: 14 R389N
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNANLIQAKKAGLQVPR
NDGDGSFDREGVAAVVRAVAVAEESRRVFRANAKKLQEIVADMACHDGYIDGFIQQLKSYKD
SEQ ID NO: 15 V66R
MASGRSSARAAGMMHVVICPWLAFGHLLPCLDLAQRLASRGHRVSFVSTPRNLSRLPPVRPALAPR
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 16 R389H
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNAHLIQAKKAGLQVPR
NDGDGSFDREGVAAVVRAVAVAEESRRVFRANAKKLQEIVADMACHDGYIDGFIQQLKSYKD
SEQ ID NO: 17 V66F
MASGRSSARAAGMMHVVICPWLAFGHLLPCLDLAQRLASRGHRVSFVSTPRNLSRLPPVRPALAPF
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 18 R389D
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NDGDGSFDREGVAAVVRAVAVAEESRRVFRANAKKLQEIVADMACHDGYIDGFIQQLKSYKD
SEQ ID NO: 19 L201N
MASGRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 20 G4N
MASNRSSARAAGMMHVVICPWLAFGHLLPCLDLAQRLASRGHRVSFVSTPRNLSRLPPVRPALAPV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKDSSGMSL
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNARLIQAKKAGLQVPR
NDGDGSFDREGVAAVVRAVAVAEESRRVFRANAKKLQEIVADMACHDGYIDGFIQQLKSYKD
SEQ ID NO: 21 Mutant 6 (P65S, V66F, V110S, R187P, D195A, L201N, G385H, R389D, D404T)
MASGRSSARAAGMMHVVICPWLAFGHLLPCLDLAQRLASRGHRVSFVSTPRNLSRLPPVRPALASF
VSFVALPLPRVEGLPDGAESTNDVPQDRPDMVELHRIAFDGLGSSFSEFLRTASADWVIVDVFHHWG
SAAAVEHKVPCAMLLLSSAHMISSISERRPESAESPAAAGEGRPAAAPTFEAAPRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNADLIQAKKAGLQVPR
NTGDGSFDREGVAAVVRAVAVAEESRRVFRANAKKLQEIVADMACHDGYIDGFIQQLKSYKD
SEQ ID NO: 22 Mutant 7 (R9S, P65S, V110S, R187P, L201N, R389D)
MASGRSSASAAGMMHVVICPWLAFGHLLPCLDLAQRLASRGHRVSFVSTPRNLSRLPPVRPALASV
VSFVALPLPRVEGLPDGAESTNDVPQDRPDMVELHRIAFDGLGSSFSEFLRTASADWVIVDVFHHWG
SAAAVEHKVPCAMLLLSSAHMISSISERRPESAESPAAAGEGRPAAAPTFEAAPRKLIRKKDSSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NDGDGSFDREGVAAVVRAVAVAEESRRVFRANAKKLQEIVADMACHDGYIDGFIQQLKSYKD
SEQ ID NO: 23 Mutant 5 (P65S, V110S, R187P, L201N, G385H, R389D, D404T)
MASGRSSARAAGMMHVVICPWLAFGHLLPCLDLAQRLASRGHRVSFVSTPRNLSRLPPVRPALASV
VSFVALPLPRVEGLPDGAESTNDVPQDRPDMVELHRIAFDGLGSSFSEFLRTASADWVIVDVFHHWG
SAAAVEHKVPCAMLLLSSAHMISSISERRPESAESPAAAGEGRPAAAPTFEAAPRKLIRKKDSSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNADLIQAKKAGLQVPR
NTGDGSFDREGVAAVVRAVAVAEESRRVFRANAKKLQEIVADMACHDGYIDGFIQQLKSYKD
SEQ ID NO: 24 Mutant 3 (G4N, R94N, D195A, L201N, G385H, R389D)
MASNRSSARAAGMMHVVICPWLAFGHLLPCLDLAQRLASRGHRVSFVSTPRNLSRLPPVRPALAPV
VSFVALPLPRVEGLPDGAESTNDVPQDNPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNADLIQAKKAGLQVPR
NDGDGSFDREGVAAVVRAVAVAEESRRVFRANAKKLQEIVADMACHDGYIDGFIQQLKSYKD
SEQ ID NO: 25 Mutant 2 (G4N, R94N, R187P, D195A, L201N, R389D, D404T)
MASNRSSARAAGMMHVVICPWLAFGHLLPCLDLAQRLASRGHRVSFVSTPRNLSRLPPVRPALAPV
VSFVALPLPRVEGLPDGAESTNDVPQDNPDMVELHRIAFDGLGVSFSEFLRTASADWVIVDVFHHWG
SAAAVEHKVPCAMLLLSSAHMISSISERRPESAESPAAAGEGRPAAAPTFEAAPRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NTGDGSFDREGVAAVVRAVAVAEESRRVFRANAKKLQEIVADMACHDGYIDGFIQQLKSYKD
SEQ ID NO: 26 Mutant 8 (R94N, R187P, L201N, R389D, D404T)
MASGRSSARAAGMMHVVICPWLAFGHLLPCLDLAQRLASRGHRVSFVSTPRNLSRLPPVRPALAPV
VSFVALPLPRVEGLPDGAESTNDVPQDNPDMVELHRIAFDGLGVSFSEFLRTASADWVIVDVFHHWG
SAAAVEHKVPCAMLLLSSAHMISSISERRPESAESPAAAGEGRPAAAPTFEAAPRKLIRKKDSSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NTGDGSFDREGVAAVVRAVAVAEESRRVFRANAKKLQEIVADMACHDGYIDGFIQQLKSYKD
SEQ ID NO: 27 Mutant 10 (G4N, V16F, R94N, V110S, L201N, R389D)
MASNRSSARAAGMMHFVICPWLAFGHLLPCLDLAQRLASRGHRVSFVSTPRNLSRLPPVRPALAPV
VSFVALPLPRVEGLPDGAESTNDVPQDNPDMVELHRIAFDGLGSSFSEFLRTASADWVIVDVFHHWG
SAAAVEHKVPCAMLLLSSAHMISSISERRPESAESPAAAGEGRPAAAPTFEAARRKLIRKKDSSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NDGDGSFDREGVAAVVRAVAVAEESRRVFRANAKKLQEIVADMACHDGYIDGFIQQLKSYKD
SEQ ID NO: 28 Mutant 9 (G4N, R9S, P65S, R187P, D195A, L201N, R389D, D404T)
MASNRSSASAAGMMHVVICPWLAFGHLLPCLDLAQRLASRGHRVSFVSTPRNLSRLPPVRPALASV
VSFVALPLPRVEGLPDGAESTNDVPQDRPDMVELHRIAFDGLGVSFSEFLRTASADWVIVDVFHHWG
SAAAVEHKVPCAMLLLSSAHMISSISERRPESAESPAAAGEGRPAAAPTFEAAPRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQGPNADLIQAKKAGLQVPR
NTGDGSFDREGVAAVVRAVAVAEESRRVFRANAKKLQEIVADMACHDGYIDGFIQQLKSYKD
SEQ ID NO: 29 Mutant 4 (R9S, R94N, D195A, L201N, G385H, R389D, D404T)
MASGRSSASAAGMMHVVICPWLAFGHLLPCLDLAQRLASRGHRVSFVSTPRNLSRLPPVRPALAPV
VSFVALPLPRVEGLPDGAESTNDVPQDNPDMVELHRIAFDGLGVSFSEFLRTASADWVIVDVFHHWG
SAAAVEHKVPCAMLLLSSAHMISSISERRPESAESPAAAGEGRPAAAPTFEAARRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNADLIQAKKAGLQVPR
NTGDGSFDREGVAAVVRAVAVAEESRRVFRANAKKLQEIVADMACHDGYIDGFIQQLKSYKD
SEQ ID NO: 30 Mutant 11 (P65S, R94N, V110S, D195A, L201N, G385H, R389D)
MASGRSSARAAGMMHVVICPWLAFGHLLPCLDLAQRLASRGHRVSFVSTPRNLSRLPPVRPALASV
VSFVALPLPRVEGLPDGAESTNDVPQDNPDMVELHRIAFDGLGSSFSEFLRTASADWVIVDVFHHWG
SAAAVEHKVPCAMLLLSSAHMISSISERRPESAESPAAAGEGRPAAAPTFEAARRKLIRKKASSGMSN
AERFFLTLSRSNLVVVRSCAELEPETVPLLSTVRGKPVAFLGLMPPSPDGRRGGVSHEGGEDDPVR
WLDAQPAESVVYVALGSEAPLLVEKVHELALGLELAGTRFLWALRKPAGVSDADLLPAGFRERTGGR
GLVATRWVPQLSILAHAAVGAFLTHCGWSSTIEGLMFGRPLIMLPISGDQHPNADLIQAKKAGLQVPR
NDGDGSFDREGVAAVVRAVAVAEESRRVFRANAKKLQEIVADMACHDGYIDGFIQQLKSYKD
SEQ ID NO: 31 Ob_UGT91B1_like
MENGSSPLHVVIFPWLAFGHLLPFLDLAERLAARGHRVSFVSTPRNLARLRPVRPALRGLVDLVALPL
PRVHGLPDGAEATSDVPFEKFELHRKAFDGLAAPFSAFLDAACAGDKRPDWVIPDFMHYWVAAAAQ
KRGVPCAVLIPCSADVMALYGQPTETSTEQPEAIARSMAAEAPSFEAERNTEEYGTAGASGVSIMTR
FSLTLKWSKLVALRSCPELEPGVFTTLTRVYSKPVVPFGLLPPRRDGAHGVRKNGEDDGAIIRWLDE
QPAKSVVYVALGSEAPVSADLLRELAHGLELAGTRFLWALRRPAGVNDGDSILPNGFLERTGERGLV
TTGWVPQVSILAHAAVCAFLTHCGWGSVVEGLQFGHPLIMLPIIGDQGPNARFLEGRKVGVAVPRNH
ADGSFDRSGVAGAVRAVAVEEEGKAFAANARKLQEIVADRERDERCTDGFIHHLTSWNELEA
SEQ ID NO: 32 Hv_UGT_v1
MDGDGNSSSSSSPLHVVICPWLALGHLLPCLDIAERLASRGHRVSFVSTPRNIARLPPLRPAVAPLVE
FVALPLPHVDGLPEGAESTNDVPYDKFELHRKAFDGLAAPFSEFLRAACAEGAGSRPDWLIVDTFHH
WAAAAAVENKVPCVMLLLGAATVIAGFARGVSEHAAAAVGKERPAAEAPSFETERRKLMTTQNASG
MTVAERYFLTLMRSDLVAIRSCAEWEPESVAALTTLAGKPVVPLGLLPPSPEGGRGVSKEDAAVRWL
DAQPAKSVVYVALGSEVPLRAEQVHELALGLELSGARFLWALRKPTDAPDAAVLPPGFEERTRGRGL
VVTGWVPQIGVLAHGAVAAFLTHCGWNSTIEGLLFGHPLIMLPISSDQGPNARLMEGRKVGMQVPRD
ESDGSFRREDVAATVRAVAVEEDGRRVFTANAKKMQEIVADGACHERCIDGFIQQLRSYKA
SEQ ID NO: 33 EUGT11
MDSGYSSSYAAAAGMHVVICPWLAFGHLLPCLDLAQRLASRGHRVSFVSTPRNISRLPPVRPALAPL
VAFVALPLPRVEGLPDGAESTNDVPHDRPDMVELHRRAFDGLAAPFSEFLGTACADWVIVDVFHHW
AAAAALEHKVPCAMMLLGSAHMIASIADRRLERAETESPAAAGQGRPAAAPTFEVARMKLIRTKGSS
GMSLAERFSLTLSRSSLVVGRSCVEFEPETVPLLSTLRGKPITFLGLMPPLHEGRREDGEDATVRWL
DAQPAKSVVYVALGSEVPLGVEKVHELALGLELAGTRFLWALRKPTGVSDADLLPAGFEERTRGRGV
VATRWVPQMSILAHAAVGAFLTHCGWNSTIEGLMFGHPLIMLPIFGDQGPNARLIEAKNAGLQVARN
DGDGSFDREGVAAAIRAVAVEEESSKVFQAKAKKLQEIVADMACHERYIDGFIQQLRSYKD
SEQ ID NO: 34 Op_UGTx5_2
MDSGYSSSAAGGMHVVICPWLAFGHLLPCLDLAQRLASRGHRVSFVSTPRNISRLPPVRPALAPLVA
FVALPLPRVEGLPDGAESTNDVPHDRPDMVELHRRAFDGLAAPFSEFLGTACADWVIVDVFHHWAA
AAALEHKVPCAMILLGSAHMVASLADRRLERAETESPAVAGQGRPAAAPTFEVARMKLIRTKGSSGM
SLAERFSLTLSRSSLVVVRSCAEFEPETVPLLSTLRGKPLAFLGLMPPSHEGRREDGEDDTVRWLDA
QPAKSVVYVALGSEVPLRVEKVHELALGLELAGTRFLWALRKPSGVSDADLLPAGFEERTRGRGVVA
TRWVPQMSILAHAAVGAFLTHCGWNSTIEGLMFGHPLIMLPIFGDQGPNARLMEAKNAGVQVPRND
GDGSFDREGVTAAIRAVAVEKESSRVFQANAKKLQVIVADMACHEGYIDGFIQQLRSYKD
SEQ ID NO: 35 Sr_UGT91D2
MATSDSIVDDRKQLHVATFPWLAFGHILPYLQLSKLIAEKGHKVSFLSTTRNIQRLSSHISPLINVVQLTL
PRVQELPEDAEATTDVHPEDIPYLKKASDGLQPEVTRFLEQHSPDWIIYDYTHYWLPSIAASLGISRAH
FSVTTPWAIAYMGPSADAMINGSDGRTTVEDLTTPPKWFPFPTKVCWRKHDLARLVPYKAPGISDGY
RMGLVLKGSDCLLSKCYHEFGTQWLPLLETLHQVPVVPVGLLPPEVPGDEKDETWVSIKKWLDGKQ
KGSVVYVALGSEVLVSQTEVVELALGLELSGLPFVWAYRKPKGPAKSDSVELPDGFVERTRDRGLV
WTSWAPQLRILSHESVCGFLTHCGSGSIVEGLMFGHPLIMLPIFGDQPLNARLLEDKQVGIEIPRNEED
GCLTKESVARSLRSVVVEKEGEIYKANARELSKIYNDTKVEKEYVSQFVDYLEKNTRAVAIDHES
SEQ ID NO: 36 UGT85C2
MDAMATTEKKPHVIFIPFPAQSHIKAMLKLAQLLHHKGLQITFVNTDFIHNQFLESSGPHCLDGAPGFR
FETIPDGVSHSPEASIPIRESLLRSIETNFLDRFIDLVTKLPDPPTCIISDGFLSVFTIDAAKKLGIPVMMY
WTLAACGFMGFYHIHSLIEKGFAPLKDASYLTNGYLDTVIDWVPGMEGIRLKDFPLDWSTDLNDKVLM
FTTEAPQRSHKVSHHIFHTFDELEPSIIKTLSLRYNHIYTIGPLQLLLDQIPEEKKQTGITSLHGYSLVKEE
PECFQWLQSKEPNSVVYVNFGSTTVMSLEDMTEFGWGLANSNHYFLWIIRSNLVIGENAVLPPELEE
HIKKRGFIASWCSQEKVLKHPSVGGFLTHCGWGSTIESLSAGVPMICWPYSWDQLTNCRYICKEWEV
GLEMGTKVKRDEVKRLVQELMGEGGHKMRNKAKDWKEKARIAIAPNGSSSLNIDKMVKEITVLARN
SEQ ID NO: 37 UGT74G1
MAEQQKIKKSPHVLLIPFPLQGHINPFIQFGKRLISKGVKTTLVTTIHTLNSTLNHSNTTTTSIEIQAISDG
CDEGGFMSAGESYLETFKQVGSKSLADLIKKLQSEGTTIDAIIYDSMTEWVLDVAIEFGIDGGSFFTQA
CVVNSLYYHVHKGLISLPLGETVSVPGFPVLQRWETPLILQNHEQIQSPWSQMLFGQFANIDQARWV
FTNSFYKLEEEVIEWTRKIWNLKVIGPTLPSMYLDKRLDDDKDNGFNLYKANHHECMNWLDDKPKES
VVYVAFGSLVKHGPEQVEEITRALIDSDVNFLWVIKHKEEGKLPENLSEVIKTGKGLIVAWCKQLDVLA
HESVGCFVTHCGFNSTLEAISLGVPVVAMPQFSDQTTNAKLLDEILGVGVRVKADENGIVRRGNLASC
IKMIMEEERGVIIRKNAVKWKDLAKVAVHEGGSSDNDIVEFVSELIKA
SEQ ID NO: 38 Sr.UGT91D_like3
MYNVTYHQNSKAMATSDSIVDDRKQLHVATFPWLAFGHILPYLQLSKLIAEKGHKVSFLSTTRNIQRLS
SHISPLINVVQLTLPRVQELPEDAEATTDVHPEDIPYLKKASDGLQPEVTRFLEQHSPDWIIYDYTHYW
LPSIAASLGISRAHFSVTTPWAIAYMGPSADAMINGSDGRTTVEDLTTPPKWFPFPTKVCWRKHDLAR
LVPYKAPGISDGYRMGLVLKGSDCLLSKCYHEFGTQWLPLLETLHQVPVVPVGLLPPEIPGDEKDET
WVSIKKWLDGKQKGSVVYVALGSEVLVSQTEVVELALGLELSGLPFVWAYRKPKGPAKSDSVELPD
GFVERTRDRGLVWTSWAPQLRILSHESVCGFLTHCGSGSIVEGLMFGHPLIMLPIFGDQPLNARLLED
KQVGIEIPRNEEDGCLTKESVARSLRSVVVEKEGEIYKANARELSKIYNDTKVEKEYVSQFVDYLEKNA
RAVAIDHES
SEQ ID NO: 39 UGT76G1
MENKTETTVRRRRRIILFPVPFQGHINPILQLANVLYSKGFSITIFHTNFNKPKTSNYPHFTFRFILDNDP
QDERISNLPTHGPLAGMRIPIINEHGADELRRELELLMLASEEDEEVSCLITDALWYFAQSVADSLNLR
RLVLMTSSLFNFHAHVSLPQFDELGYLDPDDKTRLEEQASGFPMLKVKDIKSAYSNWQILKEILGKMIK
QTKASSGVIWNSFKELEESELETVIREIPAPSFLIPLPKHLTASSSSLLDHDRTVFQWLDQQPPSSVLY
VSFGSTSEVDEKDFLEIARGLVDSKQSFLWVVRPGFVKGSTWVEPLPDGFLGERGRIVKWVPQQEV
LAHGAIGAFWTHSGWNSTLESVCEGVPMIFSDFGLDQPLNARYMSDVLKVGVYLENGWERGEIANAI
RRVMVDEEGEYIRQNARVLKQKADVSLMKGGSSYESLESLVSYISSL
SEQ ID NO: 40 UGT40087
MDASSSPLHIVIFPWLAFGHMLASLELAERLAARGHRVSFVSTPRNISRLRPVPPALAPLIDFVALPLP
RVDGLPDGAEATSDIPPGKTELHLKALDGLAAPFAAFLDAACADGSTNKVDWLFLDNFQYWAAAAAA
DHKIPCALNLTFAASTSAEYGVPRVEPPVDGSTASILQRFVLTLEKCQFVIQRACFELEPEPLPLLSDIF
GKPVIPYGLVPPCPPAEGHKREHGNAALSWLDKQQPESVLFIALGSEPPVTVEQLHEIALGLELAGTT
FLWALKKPNGLLLEADGDILPPGFEERTRDRGLVAMGWVPQPIILAHSSVGAFLTHGGWASTIEGVM
SGHPMLFLTFLDEQRINAQLIERKKAGLRVPRREKDGSYDRQGIAGAIRAVMCEEESKSVFAANAKK
MQEIVSDRNCQEKYIDELIQRLGSFEK
SEQ ID NO: 41 Bt.GGPPS
MLTSSKSIESFPKNVQPYGKHYQNGLEPVGKSQEDILLEPFHYLCSNPGKDVRTKMIEAFNAWLKVP
KDDLIVITRVIEMLHSASLLIDDVEDDSVLRRGVPAAHHIYGTPQTINCANYVYFLALKEIAKLNKPNMITI
YTDELINLHRGQGMELFWRDTLTCPTEKEFLDMVNDKTGGLLRLAVKLMQEASQSGTDYTGLVSKIGI
HFQVRDDYMNLQSKNYADNKGFCEDLTEGKFSFPIIHSIRSDPSNRQLLNILKQRSSSIELKQFALQLL
ENTNTFQYCRDFLRVLEKEAREEIKLLGGNIMLEKIMDVLSVNE
SEQ ID NO: 42 Ent-Os.CDPS
MEHARPPQGGDDDVAASTSELPYMIESIKSKLRAARNSLGETTVSAYDTAWIALVNRLDGGGERSPQ
FPEAIDWIARNQLPDGSWGDAGMFIVQDRLINTLGCVVALATWGVHEEQRARGLAYIQDNLWRLGED
DEEWMMVGFEITFPVLLEKAKNLGLDINYDDPALQDIYAKRQLKLAKIPREALHARPTTLLHSLEGMEN
LDWERLLQFKCPAGSLHSSPAASAYALSETGDKELLEYLETAINNFDGGAPCTYPVDNFDRLWSVDR
LRRLGISRYFTSEIEEYLEYAYRHLSPDGMSYGGLCPVKDIDDTAMAFRLLRLHGYNVSSSVFNHFEK
DGEYFCFAGQSSQSLTAMYNSYRASQIVFPGDDDGLEQLRAYCRAFLEERRATGNLRDKWVIANGL
PSEVEYALDFPWKASLPRVETRVYLEQYGASEDAWIGKGLYRMTLVNNDLYLEAAKADFTNFQRLSR
LEWLSLKRWYIRNNLQAHGVTEQSVLRAYFLAAANIFEPNRAAERLGWARTAILAEAIASHLRQYSAN
GAADGMTERLISGLASHDWDWRESNDSAARSLLYALDELIDLHAFGNASDSLREAWKQWLMSWTN
ESQGSTGGDTALLLVRTIEICSGRHGSAEQSLKNSEDYARLEQIASSMCSKLATKILAQNGGSMDNVE
GIDQEVDVEMKELIQRVYGSSSNDVSSVTRQTFLDVVKSFCYVAHCSPETIDGHISKVLFEDVN
SEQ ID NO: 43 Ent-Pg.KS
MKREQYTILNEKESMAEELILRIKRMFSEIENTQTSASAYDTAWVAMVPSLDSSQQPQFPQCLSWIID
NQLLDGSWGIPYLIIKDRLCHTLACVIALRKWNAGNQNVETGLRFLRENIEGIVHEDEYTPIGFQIIFPA
MLEEARGLGLELPYDLTPIKLMLTHREKIMKGKAIDHMHEYDSSLIYTVEGIHKIVDWNKVLKHQNKDG
SLFNSPSATACALMHTRKSNCLEYLSSMLQKLGNGVPSVYPINLYARISMIDRLQRLGLARHFRNEIIH
ALDDIYRYWMQRETSREGKSLTPDIVSTSIAFMLLRLHGYDVPADVFCCYDLHSIEQSGEAVTAMLSL
YRASQIMFPGETILEEIKTVSRKYLDKRKENGGIYDHNIVMKDLRGEVEYALSVPWYASLERIENRRYI
DQYGVNDTWIAKTSYKIPCISNDLFLALAKQDYNICQAIQQKELRELERWFADNKFSHLNFARQKLIYC
YFSAAATLFSPELSAARVVWAKNGVITTVVDDFFDVGGSSEEIHSFVEAVRVWDEAATDGLSENVQIL
FSALYNTVDEIVQQAFVFQGRDISIHLREIWYRLVNSMMTEAQWARTHCLPSMHEYMENAEPSIALEP
IVLSSLYFVGPKLSEEIICHPEYYNLMHLLNICGRLLNDIQGCKREAHQGKLNSVTLYMEENSGTTMED
AIVYLRKTIDESRQLLLKEVLRPSIVPRECKQLHWNMMRILQLFYLKNDGFTSPTEMLGYVNAVIVDPIL
SEQ ID NO: 44 Ps.KO
MDTLTLSLGFLSLFLFLFLLKRSTHKHSKLSHVPVVPGLPVIGNLLQLKEKKPHKTFTKMAQKYGPIFSI
KAGSSKIIVLNTAHLAKEAMVTRYSSISKRKLSTALTILTSDKCMVAMSDYNDFHKMVKKHILASVLGA
NAQKRLRFHREVMMENMSSKFNEHVKTLSDSAVDFRKIFVSELFGLALKQALGSDIESIYVEGLTATL
SREDLYNTLVVDFMEGAIEVDWRDFFPYLKWIPNKSFEKKIRRVDRQRKIIMKALINEQKKRLTSGKEL
DCYYDYLVSEAKEVTEEQMIMLLWEPIIETSDTTLVTTEWAMYELAKDKNRQDRLYEELLNVCGHEKV
TDEELSKLPYLGAVFHETLRKHSPVPIVPLRYVDEDTELGGYHIPAGSEIAINIYGCNMDSNLWENPDQ
WIPERFLDEKYAQADLYKTMAFGGGKRVCAGSLQAMLIACTAIGRLVQEFEWELGHGEEENVDTMG
LTTHRLHPLQVKLKPRNRIY
SEQ ID NO: 45 At.CPR
MSSSSSSSTSMIDLMAAIIKGEPVIVSDPANASAYESVAAELSSMLIENRQFAMIVTTSIAVLIGCIVMLV
WRRSGSGNSKRVEPLKPLVIKPREEEIDDGRKKVTIFFGTQTGTAEGFAKALGEEAKARYEKTRFKIV
DLDDYAADDDEYEEKLKKEDVAFFFLATYGDGEPTDNAARFYKWFTEGNDRGEWLKNLKYGVFGLG
NRQYEHFNKVAKVVDDILVEQGAQRLVQVGLGDDDQCIEDDFTAWREALWPELDTILREEGDTAVAT
PYTAAVLEYRVSIHDSEDAKFNDINMANGNGYTVFDAQHPYKANVAVKRELHTPESDRSCIHLEFDIA
GSGLTYETGDHVGVLCDNLSETVDEALRLLDMSPDTYFSLHAEKEDGTPISSSLPPPFPPCNLRTALT
RYACLLSSPKKSALVALAAHASDPTEAERLKHLASPAGKDEYSKWVVESQRSLLEVMAEFPSAKPPL
GVFFAGVAPRLQPRFYSISSSPKIAETRIHVTCALVYEKMPTGRIHKGVCSTWMKNAVPYEKSENCSS
APIFVRQSNFKLPSDSKVPIIMIGPGTGLAPFRGFLQERLALVESGVELGPSVLFFGCRNRRMDFIYEE
ELQRFVESGALAELSVAFSREGPTKEYVQHKMMDKASDIWNMISQGAYLYVCGDAKGMARDVHRSL
HTIAQEQGSMDSTKAEGFVKNLQTSGRYLRDVW
SEQ ID NO: 46 Sr.KAH_mutant #3
MEASYLYISILLLLASYLFTTQLRRKSANLPPTVFPSIPIIGHLYLLKKPLYRTLAKIAAKYGPILQLQLGYR
RVLVISSPSAAEECFTNNDVIFANRPKTLFGKIVGGTSLGSLSYGDQWRNLRRVASIEILSVHRLNEFH
DIRVDENRLLLRKLRDSSSPVTLRTVFYALTLNVIMRMISGKRYFDSGDRELEEEGKRFREILDETLLLA
GASNVGDYLPILNWLGVKSDEKKLIALQKKRDDFFQGLIEQVRKSRGAKVGKGRKTMIELLLSLQESE
PEYYTDAMIRSFVLGLLAAGSDTSAGTMEWAMSLLVNHPHVLKKAQAEIDRVVGNNRLIDESDIGNIP
YLGCIINETLRLYPAGPLLFPHESSADCVISGYNIPRGTMLIVNQWAIHHDPKVWDDPETFKPERFQGL
EGTRDGFKLMPFGSGRRGCPGEGLAIRLLGMTLGSVIQCFDWERVGDEMVDMTEGLGVTLPKAVPL
VAKCKPRSEMTNLLSEL

1. A variant uridine-5′-diphosphate (UDP) glycosyltransferase polypeptide comprising one or more amino acid substitutions relative to the amino acid sequence of SEQ ID NO: 1, wherein the one or more amino acid substitutions comprise an amino acid substitution at a residue selected from G4, R9, P65, V66, R94, V110, R187, D195, L201, S363, G385, R389, and D404.

2. The variant polypeptide of claim 1, wherein the one or more amino acid substitutions comprise an amino acid substitution at residue G4 of SEQ ID NO: 1.

3. The variant polypeptide of claim 2, wherein the amino acid substitution at residue G4 of SEQ ID NO: 1 substitutes G4 with an amino acid comprising a polar, uncharged side chain at physiological pH.

4. The variant polypeptide of claim 3, wherein the amino acid substitution at residue G4 of SEQ ID NO: 1 is a G4N substitution.

5. The variant polypeptide of any one of claims 1-4, wherein the one or more amino acid substitutions comprise an amino acid substitution at residue R9 of SEQ ID NO: 1.

6. The variant polypeptide of claim 5, wherein the amino acid substitution at residue R9 of SEQ ID NO: 1 substitutes R9 with an amino acid comprising a polar, uncharged side chain at physiological pH.

7. The variant polypeptide of claim 6, wherein the amino acid substitution at residue R9 of SEQ ID NO: 1 is an R9S substitution.

8. The variant polypeptide of any one of claims 1-7, wherein the one or more amino acid substitutions comprise an amino acid substitution at residue P65 of SEQ ID NO: 1.

9. The variant polypeptide of claim 8, wherein the amino acid substitution at residue P65 of SEQ ID NO: 1 substitutes P65 with an amino acid comprising a polar, uncharged side chain at physiological pH.

10. The variant polypeptide of claim 9, wherein the amino acid substitution at residue P65 of SEQ ID NO: 1 is a P65S substitution.

11. The variant polypeptide of any one of claims 1-10, wherein the one or more amino acid substitutions comprise an amino acid substitution at residue V66 of SEQ ID NO: 1.

12. The variant polypeptide of claim 11, wherein the amino acid substitution at residue V66 of SEQ ID NO: 1 substitutes V66 with an amino acid comprising a cationic side chain at physiological pH.

13. The variant polypeptide of claim 12, wherein the amino acid substitution at residue V66 of SEQ ID NO: 1 is a V66R substitution.

14. The variant polypeptide of claim 11, wherein the amino acid substitution at residue V66 of SEQ ID NO: 1 substitutes V66 with an amino acid comprising a hydrophobic, uncharged side chain at physiological pH.

15. The variant polypeptide of claim 14, wherein the amino acid substitution at residue V66 of SEQ ID NO: 1 is a V66F substitution.

16. The variant polypeptide of any one of claims 1-15, wherein the one or more amino acid substitutions comprise an amino acid substitution at residue R94 of SEQ ID NO: 1.

17. The variant polypeptide of claim 16, wherein the amino acid substitution at residue R94 of SEQ ID NO: 1 substitutes R94 with an amino acid comprising a polar, uncharged side chain at physiological pH.

18. The variant polypeptide of claim 17, wherein the amino acid substitution at residue R94 of SEQ ID NO: 1 is an R94N substitution.

19. The variant polypeptide of any one of claims 1-18, wherein the one or more amino acid substitutions comprise an amino acid substitution at residue V110 of SEQ ID NO: 1.

20. The variant polypeptide of claim 19, wherein the amino acid substitution at residue V110 of SEQ ID NO: 1 substitutes V110 with an amino acid comprising a polar, uncharged chain at physiological pH.

21. The variant polypeptide of claim 20, wherein the amino acid substitution at residue V110 of SEQ ID NO: 1 is a V110S substitution.

22. The variant polypeptide of any one of claims 1-21, wherein the one or more amino acid substitutions comprise an amino acid substitution at residue R187 of SEQ ID NO: 1.

23. The variant polypeptide of claim 22, wherein the amino acid substitution at residue R187 of SEQ ID NO: 1 is an R187P substitution.

24. The variant polypeptide of any one of claims 1-23, wherein the one or more amino acid substitutions comprise an amino acid substitution at residue D195 of SEQ ID NO: 1.

25. The variant polypeptide of claim 24, wherein the amino acid substitution at residue D195 of SEQ ID NO: 1 substitutes D195 with an amino acid comprising a hydrophobic, uncharged side chain at physiological pH.

26. The variant polypeptide of claim 25, wherein the amino acid substitution at residue D195 of SEQ ID NO: 1 is a D195A substitution.

27. The variant polypeptide of any one of claims 1-26, wherein the one or more amino acid substitutions comprise an amino acid substitution at residue L201 of SEQ ID NO: 1.

28. The variant polypeptide of claim 27, wherein the amino acid substitution at residue L201 of SEQ ID NO: 1 substitutes L201 with an amino acid comprising a polar, uncharged side chain at physiological pH.

29. The variant polypeptide of claim 28, wherein the amino acid substitution at residue L201 of SEQ ID NO: 1 is an L201N substitution.

30. The variant polypeptide of any one of claims 1-29, wherein the one or more amino acid substitutions comprise an amino acid substitution at residue S363 of SEQ ID NO: 1.

31. The variant polypeptide of claim 30, wherein the amino acid substitution at residue S363 of SEQ ID NO: 1 substitutes S363 with an amino acid comprising a polar, uncharged side chain at physiological pH.

32. The variant polypeptide of claim 31, wherein the amino acid substitution at residue S363 of SEQ ID NO: 1 is an S363N substitution.

33. The variant polypeptide of any one of claims 1-32, wherein the one or more amino acid substitutions comprise an amino acid substitution at residue G385 of SEQ ID NO: 1.

34. The variant polypeptide of claim 33, wherein the amino acid substitution at residue G385 of SEQ ID NO: 1 substitutes G385 with an amino acid comprising a cationic side chain at physiological pH.

35. The variant polypeptide of claim 34, wherein the amino acid substitution at residue G385 of SEQ ID NO: 1 is a G385H substitution.

36. The variant polypeptide of claim 33, wherein the amino acid substitution at residue G385 of SEQ ID NO: 1 substitutes G385 with an amino acid comprising a hydrophobic, uncharged side chain at physiological pH.

37. The variant polypeptide of claim 36, wherein the amino acid substitution at residue G385 of SEQ ID NO: 1 is a G3851 substitution.

38. The variant polypeptide of any one of claims 1-37, wherein the one or more amino acid substitutions comprise an amino acid substitution at residue R389 of SEQ ID NO: 1.

39. The variant polypeptide of claim 38, wherein the amino acid substitution at residue R389 of SEQ ID NO: 1 substitutes R389 with an amino acid comprising a cationic side chain at physiological pH.

40. The variant polypeptide of claim 39, wherein the amino acid substitution at residue R389 of SEQ ID NO: 1 is an R389H substitution.

41. The variant polypeptide of claim 38, wherein the amino acid substitution at residue R389 of SEQ ID NO: 1 substitutes R389 with an amino acid comprising an anionic side chain at physiological pH.

42. The variant polypeptide of claim 41, wherein the amino acid substitution at residue R389 of SEQ ID NO: 1 is an R389D substitution.

43. The variant polypeptide of claim 38, wherein the amino acid substitution at residue R389 of SEQ ID NO: 1 substitutes R389 with an amino acid comprising a polar, uncharged side chain at physiological pH.

44. The variant polypeptide of claim 43, wherein the amino acid substitution at residue R389 of SEQ ID NO: 1 is an R389N substitution.

45. The variant polypeptide of claim 38, wherein the amino acid substitution at residue R389 of SEQ ID NO: 1 substitutes R389 with an amino acid comprising a hydrophobic, uncharged side chain at physiological pH.

46. The variant polypeptide of claim 45, wherein the amino acid substitution at residue R389 of SEQ ID NO: 1 is an R389F substitution.

47. The variant polypeptide of any one of claims 1-46, wherein the one or more amino acid substitutions comprise an amino acid substitution at residue D404 of SEQ ID NO: 1.

48. The variant polypeptide of claim 47, wherein the amino acid substitution at residue D404 of SEQ ID NO: 1 substitutes D404 with an amino acid comprising a polar, uncharged chain at physiological pH.

49. The variant polypeptide of claim 48, wherein the amino acid substitution at residue D404 of SEQ ID NO: 1 is a D404T substitution.

50. The variant polypeptide of claim 48, wherein the amino acid substitution at residue D404 of SEQ ID NO: 1 is a D404S substitution.

51. The variant polypeptide of any one of claims 1-50, wherein the one or more amino acid substitutions comprise P65S, V66F, V110S, R187P, D195A, L201N, G385H, R389D, and D404T relative to SEQ ID NO: 1.

52. The variant polypeptide of any one of claims 1-50, wherein the one or more amino acid substitutions comprise R9S, P65S, V110S, R187P, L201N, and R389D relative to SEQ ID NO: 1.

53. The variant polypeptide of any one of claims 1-50, wherein the one or more amino acid substitutions comprise P65S, V110S, R187P, L201N, G385H, R389D, and D404T relative to SEQ ID NO: 1.

54. The variant polypeptide of any one of claims 1-50, wherein the one or more amino acid substitutions comprise G4N, R94N, D195A, L201N, G385H, and R389D relative to SEQ ID NO: 1.

55. The variant polypeptide of any one of claims 1-50, wherein the one or more amino acid substitutions comprise G4N, R94N, R187P, D195A, L201N, R389D, and D404T relative to SEQ ID NO: 1.

56. The variant polypeptide of any one of claims 1-50, wherein the one or more amino acid substitutions comprise R94N, R187P, L201N, R389D, and D404T relative to SEQ ID NO: 1.

57. The variant polypeptide of any one of claims 1-50, wherein the one or more amino acid substitutions comprise G4N, V16F, R94N, V110S, L201N, and R389D relative to SEQ ID NO: 1.

58. The variant polypeptide of any one of claims 1-50, wherein the one or more amino acid substitutions comprise G4N, R9S, P65S, R187P, D195A, L201N, R389D, and D404T relative to SEQ ID NO: 1.

59. The variant polypeptide of any one of claims 1-50, wherein the one or more amino acid substitutions comprise R9S, R94N, D195A, L201N, G385H, R389D, and D404T relative to SEQ ID NO: 1.

60. The variant polypeptide of any one of claims 1-50, wherein the one or more amino acid substitutions comprise P65S, R94N, V110S, D195A, L201N, G385H, and R389D relative to SEQ ID NO: 1.

61. The variant polypeptide of any one of claims 1-60, wherein the polypeptide has an amino acid sequence that is from about 85% to about 99.7% identical to the amino acid sequence of SEQ ID NO:
1.

62. The variant polypeptide of claim 61, wherein the polypeptide has an amino acid sequence that is from about 90% to about 99.7% identical to the amino acid sequence of SEQ ID NO: 1.

63. The variant polypeptide of any one of claims 1-62, wherein the polypeptide has an amino acid sequence that differs from the amino acid sequence of SEQ ID NO: 1 only by way of (i) the one or more amino acid substitutions or deletions and, optionally, (ii) one or more additional, conservative amino acid substitutions.

64. The variant polypeptide of claim 63, wherein the polypeptide has an amino acid sequence that differs from the amino acid sequence of SEQ ID NO: 1 only by way of the one or more amino acid substitutions or deletions.

65. The variant polypeptide of any one of claims 1-64, wherein the polypeptide has an amino acid sequence that is at least 85% identical to the amino acid sequence of any one of SEQ ID NO: 2-30.

66. The variant polypeptide of claim 65, wherein the polypeptide has an amino acid sequence that is at least 90% identical to the amino acid sequence of any one of SEQ ID NO: 2-30.

67. The variant polypeptide of claim 66, wherein the polypeptide has an amino acid sequence that is at least 95% identical to the amino acid sequence of any one of SEQ ID NO: 2-30.

68. The variant polypeptide of claim 67, wherein the polypeptide has the amino acid sequence of any one of SEQ ID NO: 2-30.

69. The variant polypeptide of any one of claims 1-68, wherein the polypeptide catalyzes glycosylation at the 2′ position of the 13-O-glucose of a steviol glycoside, optionally wherein the polypeptide exhibits increased glycosylation activity at the 2′ position of the 13-O-glucose of a steviol glycoside as compared to a polypeptide having the amino acid sequence of SEQ ID NO: 1.

70. The variant polypeptide of claim 69, wherein the polypeptide exhibits at least a 1.1-fold increase in glycosylation activity at the 2′ position of the 13-O-glucose of a steviol glycoside as compared to a polypeptide having the amino acid sequence of SEQ ID NO: 1.

71. The variant polypeptide of claim 69, wherein the polypeptide exhibits between a 1.1-fold and 10-fold increase in glycosylation activity at the 2′ position of the 13-O-glucose of a steviol glycoside as compared to a polypeptide having the amino acid sequence of SEQ ID NO: 1.

72. A nucleic acid encoding the variant polypeptide of any one of claims 1-71.

73. A host cell comprising the variant polypeptide of any one of claims 1-71 or the nucleic acid of claim 72.

74. The host cell of claim 73, wherein the nucleic acid encoding the variant polypeptide is integrated into the genome of the cell.

75. The host cell of claim 73, wherein the nucleic acid encoding the variant polypeptide is present within a plasmid.

76. A host cell capable of producing one or more steviol glycosides, wherein the host cell comprises one or more heterologous nucleic acids that each, independently, encode a UDP glycosyltransferase having an amino acid sequence that is at least 90% identical to the amino acid sequence of any one of SEQ ID NO: 2-30.

77. The host cell of claim 76, wherein the host cell comprises one or more heterologous nucleic acids that each, independently, encode a UDP glycosyltransferase having an amino acid sequence that is at least 95% identical to the amino acid sequence of any one of SEQ ID NO: 2-30.

78. The host cell of claim 77, wherein the glycosyltransferase has the amino acid sequence of any one of SEQ ID NO: 2-30.

79. The host cell of any one of claims 73-78, wherein the host cell comprises one or more heterologous nucleic acids encoding a geranylgeranyl diphosphate synthase (GGPPS), a copalyl diphosphate synthase (CDPS), a kaurene synthase (KS), a kaurene oxidase (KO), a kaurene acid hydroxylase (KAH), a cytochrome P450 reductase (CPR), and one or more UDP glycosyltransferases.

80. The host cell of any one of claims 73-79, wherein the host cell comprises a heterologous nucleic acid encoding a GGPPS.

81. The host cell of claim 80, wherein the GGPPS has an amino acid sequence that is at least 90% identical to the amino acid sequence of SEQ ID NO: 41.

82. The host cell of claim 81, wherein the GGPPS has an amino acid sequence that is at least 95% identical to the amino acid sequence of SEQ ID NO: 41.

83. The host cell of claim 82, wherein the GGPPS has the amino acid sequence of SEQ ID NO: 41.

84. The host cell of any one of claims 73-83, wherein the host cell comprises a heterologous nucleic acid encoding a CDPS.

85. The host cell of claim 84, wherein the CDPS has an amino acid sequence that is at least 90% identical to the amino acid sequence of SEQ ID NO: 42.

86. The host cell of claim 85, wherein the CDPS has an amino acid sequence that is at least 95% identical to the amino acid sequence of SEQ ID NO: 42.

87. The host cell of claim 86, wherein the CDPS has the amino acid sequence of SEQ ID NO: 42.

88. The host cell of any one of claims 73-87, wherein the host cell comprises a heterologous nucleic acid encoding a KS.

89. The host cell of claim 88, wherein the KS has an amino acid sequence that is at least 90% identical to the amino acid sequence of SEQ ID NO: 43.

90. The host cell of claim 89, wherein the KS has an amino acid sequence that is at least 95% identical to the amino acid sequence of SEQ ID NO: 43.

91. The host cell of claim 90, wherein the KS has the amino acid sequence of SEQ ID NO: 43.

92. The host cell of any one of claims 73-91, wherein the host cell comprises a heterologous nucleic acid encoding a KO.

93. The host cell of claim 92, wherein the KO has an amino acid sequence that is at least 90% identical to the amino acid sequence of SEQ ID NO: 44.

94. The host cell of claim 93, wherein the KO has an amino acid sequence that is at least 95% identical to the amino acid sequence of SEQ ID NO: 44.

95. The host cell of claim 94, wherein the KO has the amino acid sequence of SEQ ID NO: 44.

96. The host cell of any one of claims 73-95, wherein the host cell comprises a heterologous nucleic acid encoding a KAH.

97. The host cell of claim 96, wherein the KAH has an amino acid sequence that is at least 90% identical to the amino acid sequence of SEQ ID NO: 46.

98. The host cell of claim 97, wherein the KAH has an amino acid sequence that is at least 95% identical to the amino acid sequence of SEQ ID NO: 46.

99. The host cell of claim 98, wherein the KAH has the amino acid sequence of SEQ ID NO: 46.

100. The host cell of any one of claims 73-99, wherein the host cell comprises a heterologous nucleic acid encoding a CPR.

101. The host cell of claim 100, wherein the CPR has an amino acid sequence that is at least 90% identical to the amino acid sequence of SEQ ID NO: 45.

102. The host cell of claim 101, wherein the CPR has an amino acid sequence that is at least 95% identical to the amino acid sequence of SEQ ID NO: 45.

103. The host cell of claim 102, wherein the CPR has the amino acid sequence of SEQ ID NO: 45.

104. The host cell of any one of claims 73-103, wherein the host cell comprises one or more heterologous nucleic acids encoding one or more additional UDP glycosyltransferases, optionally wherein the one or more additional UDP glycosyltransferases are selected from a UGT74G1, a UGT85C2, a UGT40087, and a UGT76G1.

105. The host cell of claim 104, wherein the host cell comprises a heterologous nucleic acid encoding a UGT74G1.

106. The host cell of claim 105, wherein the UGT74G1 has an amino acid sequence that is at least 90% identical to the amino acid sequence of SEQ ID NO: 37.

107. The host cell of claim 106, wherein the UGT74G1 has an amino acid sequence that is at least 95% identical to the amino acid sequence of SEQ ID NO: 37.

108. The host cell of claim 107, wherein the UGT74G1 has the amino acid sequence of SEQ ID NO: 37.

109. The host cell of any one of claims 104-108, wherein the host cell comprises a heterologous nucleic acid encoding a UGT85C2.

110. The host cell of claim 109, wherein the UGT85C2 has an amino acid sequence that is at least 90% identical to the amino acid sequence of SEQ ID NO: 36.

111. The host cell of claim 110, wherein the UGT85C2 has an amino acid sequence that is at least 95% identical to the amino acid sequence of SEQ ID NO: 36.

112. The host cell of claim 111, wherein the UGT85C2 has the amino acid sequence of SEQ ID NO: 36.

113. The host cell of any one of claims 104-112, wherein the host cell comprises a heterologous nucleic acid encoding a UGT40087.

114. The host cell of claim 113, wherein the UGT40087 has an amino acid sequence that is at least 90% identical to the amino acid sequence of SEQ ID NO: 40.

115. The host cell of claim 114, wherein the UGT40087 has an amino acid sequence that is at least 95% identical to the amino acid sequence of SEQ ID NO: 40.

116. The host cell of claim 115, wherein the UGT40087 has the amino acid sequence of SEQ ID NO: 40.

117. The host cell of any one of claims 104-116, wherein the host cell comprises a heterologous nucleic acid encoding a UGT76G1.

118. The host cell of claim 117, wherein the UGT76G1 has an amino acid sequence that is at least 90% identical to the amino acid sequence of SEQ ID NO: 39.

119. The host cell of claim 118, wherein the UGT76G1 has an amino acid sequence that is at least 95% identical to the amino acid sequence of SEQ ID NO: 39.

120. The host cell of claim 119, wherein the UGT76G1 has the amino acid sequence of SEQ ID NO: 39.

121. The host cell of any one of claims 76-120, wherein the one or more heterologous nucleic acids are present within one or more plasmids in the host cell.

122. The host cell of any one of claims 76-120, wherein the one or more heterologous nucleic acids are integrated into the genome of the host cell.

123. The host cell of any one of claims 76-122, wherein the one or more steviol glycosides are selected from RebA, RebB, RebD, RebE, and RebM.

124. The host cell of claim 123, wherein the one or more steviol glycosides comprise RebM.

125. The host cell of any one of claims 73-124, wherein the host cell is selected from a bacterial cell, a yeast cell, an algal cell, an insect cell, and a plant cell.

126. The host cell of claim 125, wherein the host cell is a yeast cell.

127. The host cell of claim 126, wherein the yeast cell is Saccharomyces cerevisiae.

128. A method for producing one or more steviol glycosides comprising:
culturing a population of host cells of any one of claims 73-127 in a medium with a carbon source under conditions suitable for making one or more steviol glycosides, thereby yielding a culture broth; and
recovering the one or more steviol glycosides from the culture broth.

129. The method of claim 128, wherein the one or more steviol glycosides are selected from RebA, RebB, RebD, RebE, and RebM, optionally wherein the one or more steviol glycosides comprise RebM.

130. A fermentation composition comprising:
(i) a population of host cells of any one of claims 73-127, and (ii) one or more steviol glycosides produced by the host cell.

131. The fermentation composition of claim 130, wherein the one or more steviol glycosides are selected from RebA, RebB, RebD, RebE, and RebM, optionally wherein the one or more steviol glycosides comprise RebM.

132. A composition comprising a steviol glycoside produced by the method of claim 128 or 129.

133. The composition of claim 132, wherein the steviol glycoside is selected from RebA, RebB, RebD, RebE, and RebM, optionally wherein the steviol glycoside is RebM.
